(19) United States
(12) Patent Application Publication
(10) Pub. No.: US 2005/0149090 A1
(43) Pub. Date: Jul. 7, 2005

(54) Lancet cassette and lancet ejecting device, and lancet assembly composed of them

(76) Inventors: Susumu Morita, Hyogo-ken (JP); Tomoyuki Hyoue, Okayama-ken (JP)

Correspondence Address: WENDEROTH, LIND & PONACK, L.L.P., 2033 K STREET N. W., SUITE 800, WASHINGTON, DC, 20006-1021, US

(21) Appl. No.: 10/959,085

(22) Filed: Oct. 7, 2004

(30) Foreign Application Priority Data
Oct. 10, 2003 (JP) 2003-352206

(51) Int. Cl. A61B 17/14

(52) U.S. Cl. 606/181

(57) ABSTRACT

There is provided a lancet assembly which is improved in view points of its operation, convenience and safety. A lancet cassette which includes a lancet having a pricking member for piercing a predetermined portion and a container having a space in which the lancet moves, characterized in that the lancet cassette is capable of mounting a thin sensor element thereon, and a moving direction of the pricking member of the lancet intersects with an extending plane of the sensor element in the vicinity of a leading end of the sensor element. Such cassette is used to form a lancet assembly.

### CROSS-REFERENCE TO RELATED APPLICATION

The present application claims a priority under the Paris Convention based on Japanese Patent Application No. 2003-352206 (filed on Oct. 10, 2003, title of invention: lancet cassette and lancet ejecting device, and lancet assembly composed of them). The contents of that application are incorporated herein by reference in their entirety.

### FIELD OF THE INVENTION

The present invention relates to a lancet assembly which comprises a cassette including a lancet and a lancet ejecting device. Such assembly ejects the lancet, and thus it is also referred to as a “lancet ejecting assembly”. Such assembly can be used for obtaining a small amount of blood from a predetermined portion (for example, a tip of a finger) of a human, an animal or the like. In a preferable embodiment, the cassette comprises a sensor for blood glucose level measurement, and the lancet ejecting device comprises a unit for blood glucose level measurement, so that the assembly according to the present invention allows both of the blood collection and the blood glucose level measurement of the collected blood.

### BACKGROUND ART

A blood glucose level measuring system is currently used wherein a small amount blood collecting device (that is, a lancet and a pricking device) and a blood glucose level measuring device for the collected blood (that is, a sensor and a blood glucose level measuring device) which two devices are separate from each other are to be used in combination so as to measure a blood glucose level of a diabetes patient.

For example, Accu-Chek Comfort from Roche (Swizerland), One Touch Ultra from Lifescan (U.S.), Dexter ZII from Bayer (Germany) and Dia-Meter α from Arkray (Japan) are commercially available as the blood glucose level measuring device. With the blood glucose level measuring system in which the above described blood glucose level measuring device is used, other separate small amount blood collecting device is required, and such system is mainly used domestically. As such separate small amount blood collecting device, Softclix and Softclix lancet from Roche, One Touch Ultra Soft and One Touch Ultra Soft lancet from Lifescan, Microlet and Microlet lancet from Bayer and Multilancet II and Multilet are commercially available.

In hospitals, blood glucose level measurement is carried out as to general public patients, and there is a risk that an amount of blood having deposited to a pricking device upon the blood collection of a previous patient may attach to a pricking portion of the next patient, so that the next patient may be infectious disease (such as AIDS, hepatitis B, hepatitis C or the like) by means of thus attaching blood. Due to such situation, a single use safety lancet which cannot be reused is used for the blood collection as a pricking device. The measurement of the blood glucose level is carried out using the blood glucose level measuring device as described above.

Recently, the measurement of the blood glucose level is required in animal hospitals. Since there is no commercially available system which is specific for animals, the small amount blood collecting device as described above is used for the animals.

In the above mentioned prior art, the two devices, namely the device for the blood collection and the device for the measurement are required, so that the measurement requires a large amount of labor, and as to the conveniences of such operationality, portability or the like, those devices are not completely satisfactory.

For example, when a blood glucose level measuring system is used in which a blood glucose level measuring device such as One Touch Ultra from Lifescan as well as a small amount blood collecting device such as One Touch Ultra Soft and One Touch Ultra Soft lancet from Lifescan are used in combination, the following operations are required:
for the preparation of the blood collection using the blood collecting device, (1) preparing a pricking device, (2) removing a front cap from the device, (3) fitting a lancet into a lancet holder of the device, (4) twisting off and removing a needle tip shielding cap from the lancet, (5) placing on the pricking device the front cap which has been removed, and (6) pulling a cocking lever of the pricking device backward to be for the ready state; and
also, for the preparation of the measurement using the measuring device, (7) preparing a measurement sensor (i.e. test paper), and (8) inserting the sensor into the measuring device;
then, (9) taking up the pricking device which is in the ready state, (10) holding a blood collection portion against a tip portion of the pricking device, (11) pressing a lancet ejecting button so as to eject the lancet and piercing the blood collection portion, (12) confirming bleeding and putting the pricing device, (13) spotting an amount of the bleeding blood on the sensor in the device, (14) reading an indicated measurement, (15) removing the sensor from the device and disposing the sensor, and (16) returning the measuring device to its original state;
as to the pricking device, (17) removing the front cap, (18) placing a safety cap on the lancet which is fitted into the lancet holder, (19) removing the lancet from the lancet holder and disposeing the lancet, (20) placing the front cap on the pricking device, (21) returning the pricking device to its original state.

A system is commercially available which intends to alleviate the above described problem as to the conveniences. For example, Soft Tact from Abbott (U.S.) and Medi-Safe eZ from Terumo (Japan) are commercially available. These systems allow the blood collection and the measurement to be carried out in a single unit so that their conveniences are improved. However, in these systems, the pricking device and the measuring device are merely mechanically integrated.

Apart from such systems, WO02/56769 discloses a unit in which a sensor which includes a lancet therein, and such sensor is integrated with a blood glucose level measuring device. With this unit, there is a possibility that a needle is accidentally exposed from a front end of the sensor after using the lancet, which causes a safety related problem.

### DISCLOSURE OF THE INVENTION

It is therefore desirable that a lancet assembly is provided which is further improved in its operability, convenience and safety.

The present invention provides a lancet cassette comprising a lancet which includes a pricking member for piercing a predetermined portion and a container which defines a space therein where the lancet moves, characterized in that
the lancet cassette is capable of mounting a thin sensor element thereon, and
a moving direction of the pricking member of the lancet intersects with an extending plane of the sensor element in the vicinity of a leading end of the sensor element.

Also, the present invention provides a lancet assembly comprising the lancet cassette as described above and a lancet ejecting device which comprises a lancet ejecting member and ejects the lancet, and the pricking member pierce the predetermined portion.

In addition, the present invention provides the lancet ejecting device which forms, in combination with the lancet cassette as described above, the above described lancet assembly.

In the present specification, the terms “front” and “rear” as well as the phrases which substantially include meanings of such terms (for example, “forward” and “front end”, and “backward” and “rear end”) are used based on a direction along which the lancet moves so as to prick (or pierce) the predetermined portion (which is also hereinafter referred to as a “pricking direction” corresponding to a direction indicated with arrow A in FIG. 17). Such pricking direction forms an angle with the horizontal plane (which is defined as the extending plane of the sensor element as explained later and which corresponds to a plane indicated with B in FIG. 17), that is, the pricking direction is inclined relative to the horizontal plane, and an extent of such inclination, namely the angle formed with the horizontal plane is relatively small.

Also, in the present specification, the terms “up(per)” and “low(er)” as well as the phrases which substantially include meanings of such terms (for example, “upward” and “upper side”, and “downward” and “lower side”) are used based on the horizontal plane as described above unless otherwise mentioned.

The present invention will be hereinafter explained in detail, and merely for the purpose of easily understanding, reference numbers are given to concrete features which correspond to the elements constituting the present inventions, but it is to be understood that such reference numbers do not intend to limit the elements which constitute the present inventions to such concrete features.

### BRIEF DESCRIPTION OF THE DRAWINGS

FIG. 1 schematically shows a perspective view of a lancet cassette and a lancet ejecting device according to the present invention which are completely exploded.

FIG. 2 schematically shows a perspective view of the lancet cassette according to the present invention in its assembled state and the lancet ejecting device according to the present invention in which an engaging portion holding member, a stopper, an ejection member and a trigger member are incorporated in a housing lower half of the exploded state shown in FIG. 1.

FIG. 3 schematically shows a perspective view of the lancet ejecting device according to the present invention in which an connector and a cover member are incorporated in the housing lower half in the state shown in FIG. 2.

FIG. 4 schematically shows a perspective view of the lancet ejecting device according to the present invention in which an electronic device plate having an electronic device mounted thereon is incorporated in the housing lower half in the state shown in FIG. 3.

FIG. 5 schematically shows a perspective view of the lancet ejecting device according to the present invention in which a pressing member is incorporated in the housing lower half in the state shown in FIG. 4. It is noted that a discharge button and an elastic member are attached to an housing upper half.

FIG. 6 schematically shows a perspective view of a completed lancet ejecting device according to the present invention in which the housing upper half is incorporated with the housing lower half in the state shown in FIG. 5.

FIG. 7 schematically shows a perspective view of the completed lancet ejecting device according to the present invention shown in FIG. 6 into which the lancet cassette has been fitted.

FIG. 8 schematically shows a perspective view of a container preform after its molding which is used for forming the container of the lancet cassette according to the present invention.

FIG. 9 schematically shows a perspective view of a state where an unused lancet is placed in the state shown in FIG. 8.

FIG. 10 schematically shows a perspective view of a state where for the production of the cassette, an upper half is rotated around a hinge of the preform by about 90 degrees in the state shown in FIG. 9.

FIG. 11 schematically shows a perspective view of a state where for the production of the cassette, the upper half is further rotated around the hinge of the preform in the state shown in FIG. 10.

FIG. 12 schematically shows a perspective view of a state where the upper half is further rotated around the hinge of the preform in the state shown in FIG. 11 so that the upper half is substantially stacked on a lower half to complete the cassette.

FIG. 13 schematically shows a perspective view of a state where a sensor element is being placed on an outside surface of the upper half of the container.

FIG. 14 schematically shows a perspective view of a state where the sensor element has been placed on the outside surface of the upper half of the container, that is, a perspective view of the lancet cassette comprising the sensor element.

FIG. 15 schematically shows a perspective view of a lancet which is housed in the lancet cassette according to the present invention.

FIG. 16 schematically shows a side elevation of the lancet shown in FIG. 15.

FIG. 17 schematically shows a state by means of a cross-sectional view of the cassette wherein a moving direction of a pricking member intersects with an extending plane of the sensor element in the lancet cassette according to the present invention, and a finger tip is placed against a front end of the cassette.

FIG. 18 schematically shows, in a perspective view, a state wherein an unused lancet having a shielding member is housed in the lancet cassette according to the present invention with near side halves of the upper half and the sensor element being cut away for the purpose of seeing the inside of the container.

FIG. 19 is a perspective view similar to FIG. 18, in which perspective view a tip portion of the pricking member is exposed by removing the shielding member from the lancet in the state of FIG. 18.

FIG. 20 is a perspective view similar to FIG. 18, in which perspective view the lancet has been ejected from the state shown in FIG. 19, so that the tip portion of the pricking member is protruding from a front end of the container while retracting portions hit against collision portions of the container and thereby the retracting portions are bent.

FIG. 21 is a perspective view similar to FIG. 18, in which perspective view the bent portions have returned to their original forms so that tip portion of the pricking member has been retracted into the container while a lancet rear end and hook portions have been received in the container.

FIG. 22 schematically shows a side elevation of the lancet cassette shown in FIG. 18.

FIG. 23 schematically shows a side elevation of the lancet cassette shown in FIG. 19.

FIG. 24 schematically shows a side elevation of the lancet cassette shown in FIG. 20.

FIG. 25 schematically shows a perspective view of a connector when it is seen from its front side.

FIG. 26 schematically shows a perspective view of the connector shown in FIG. 25 when it is turned upside down.

FIG. 27 schematically shows a perspective view of the connector when it is seen from its rear side.

FIG. 28 schematically shows a perspective view of the connector shown in FIG. 27 when it is turned upside down.

FIG. 29 schematically shows a front view of the connector when it is seen from its front side.

FIG. 30 schematically shows, in a perspective view, a state wherein the connector is loaded with the cassette, and the lancet and the sensor element are not shaded while the connector and the container are differently shaded for their easy distinction.

FIG. 31 schematically shows, in a perspective view, a state wherein the state shown in FIG. 30 is turned upside down.

FIG. 32 schematically shows a perspective view of the lancet ejecting member.

FIG. 33 schematically shows the lancet ejecting device when the housing lower half is seen from its upper side while the housing upper half, the electronic device plate and the cover member are excluded (that is, a plan view of the housing lower half as shown in the lower part of FIG. 3 while removing the cover member).

FIG. 34 schematically shows, in a side elevation, a positional relationship among the lancet cassette, the connector, the stopper, the ejecting member (only its leading end portion is shown) and the trigger member (only its leading end portion is shown) in the state as shown in FIG. 33.

FIG. 35 shows, in a plan view similar to FIG. 33, the housing lower half wherein the cassette is in a halfway state when it is inserted in the connector in the state shown in FIG. 33, and a rear end of the cassette is in contact with an engaging portion.

FIG. 36 shows a side elevation similarly to FIG. 34, which shows the state of FIG. 35.

FIG. 37 schematically shows a perspective view which corresponds to the side elevation of FIG. 36. It is noted that the container is shown with its near side portion being cut away so that the state of the inside of the container can be seen.

FIG. 38 shows, in a plan view similar to FIG. 33, the housing lower half wherein after the state shown in FIG. 35, the cassette is further inserted in the connector, so that the engaging portion is being pressed downward.

FIG. 39 shows a side elevation similarly to FIG. 34, which shows the state of FIG. 38.

FIG. 40 schematically shows a perspective view which corresponds to the side elevation of FIG. 39. It is noted that the connector and the trigger member are shown with their near side portions being cut away.

FIG. 41 shows, in a plan view similar to FIG. 33, the housing lower half wherein after the state shown in FIG. 38, the cassette is further inserted in the connector, so that the engaging portion is just before it fits into a concave portion of the container.

FIG. 42 shows a side elevation similarly to FIG. 34, which shows the state of FIG. 41.

FIG. 43 schematically shows a perspective view which corresponds to the side elevation of FIG. 42. It is noted that the connector and the trigger member are shown with their near side portions being cut away.

FIG. 44 shows a perspective view similarly to FIG. 43 except that the container is omitted and the trigger member is entirely shown.

FIG. 45 schematically shows a perspective view of the device in the state as shown in FIG. 44 when it is seen from its lower side.

FIG. 46 shows, in a plan view similar to FIG. 33, the housing lower half wherein after the state shown in FIG. 41, the cassette is further inserted in the connector, so that the engaging portion is fitting into the concave portion of the container.

FIG. 47 shows a side elevation similarly to FIG. 34, which shows the state of FIG. 46.

FIG. 48 schematically shows a perspective view which corresponds to the side elevation of FIG. 47. It is noted that the connector and the trigger member are shown with their near side portions being cut away.

FIG. 49 schematically shows a perspective view of the device in the state as shown in FIG. 48 when it is seen from its lower side.

FIG. 50 shows, in a plan view similar to FIG. 33, the housing lower half wherein after the state shown in FIG. 46, the shielding member has been removed from the lancet.

FIG. 51 shows a side elevation similarly to FIG. 34, which shows the state of FIG. 50.

FIG. 52 shows, in a plan view similar to FIG. 33, the housing lower half wherein after the state shown in FIG. 50, the trigger member is pressed forward, so that its leading end portions abut against lancet hook portions.

FIG. 53 shows a side elevation similarly to FIG. 34, which shows the state of FIG. 52.

FIG. 54 shows, in a plan view similar to FIG. 33 the housing lower half wherein after the state shown in FIG. 52, the trigger member is further pressed forward so as to deform the lancet hook portions such that they are directed to a center axis of the lancet, so that the lancet is in a state just after its ejection has started.

FIG. 55 shows a side elevation similarly to FIG. 34, which shows a state of FIG. 54.

FIG. 56 shows, in a plan view similar to FIG. 33, the housing lower half wherein after the state shown in FIG. 54, the retracting portions of the lancet which moves forward are in a state just after they hit against collision portions.

FIG. 57 shows a side elevation similarly to FIG. 34, which shows the state of FIG. 56.

FIG. 58 shows, in a plan view similar to FIG. 33, the housing lower half wherein after the state shown in FIG. 56, the retracting portions of the lancet are bent by hitting against collision portions.

FIG. 59 shows a side elevation similarly to FIG. 34, which shows the state of FIG. 58. It is noted that an angle α and an angle β are shown for their understanding.

FIG. 60 shows, in a plan view similar to FIG. 33, the housing lower half wherein after the state shown in FIG. 58, the tip portion of the pricking member is exposed from the opening at the front end of the container.

FIG. 61 shows a side elevation similarly to FIG. 34, which shows the state of FIG. 60.

FIG. 62 shows a perspective view which corresponds to the side elevation of FIG. 61. It is noted that the container is omitted and a near side portion of the connecter is cut away.

FIG. 63 schematically shows, in a perspective view, the device which is shown in the perspective view in FIG. 62, when the device is seen from its lower side.

FIG. 64 shows, in a plan view similar to FIG. 33, the housing lower half wherein after the state shown in FIG. 60, the tip portion of the pricking member which has been exposed is retracted into inside of the container by return of the retracting portions to their original forms.

FIG. 65 shows a side elevation similarly to FIG. 34, which shows the state of FIG. 64.

FIG. 66 shows, in a plan view similar to FIG. 33, the housing lower half wherein after the state shown in FIG. 64, the trigger member has been moved back by releasing a force which pushes the trigger member forward.

FIG. 67 shows a side elevation similarly to FIG. 34, which shows the state of FIG. 66. It is noted that with the return of the trigger member, also the ejecting member has been moved back and returned to its original position at the time before the cassette was charged (for example, see FIG. 34), and a protruding portion of the ejecting member is located below the rear end of the lancet.

FIG. 68 shows, in a plan view similar to FIG. 33, the housing lower half wherein the lancet ejecting device according to the present invention is completely loaded with a cassette including a spent lancet.

FIG. 69 shows a side elevation similarly to FIG. 34, which shows the state of FIG. 68. It is obviously seen that an edge portion of the rear end of the lancet is in contact with a protruding portion of the lancet ejecting member, but the lancet ejecting member cannot be moved further backward even though completely loaded with the cassette, and therefore the spent lancet is never ejected.

FIG. 70 schematically shows an elevation view of the stopper which is used in the lancet ejecting device. In the shown embodiment, no force is acting on an oblique portion of the stopper.

FIG. 71 schematically shows an elevation view of the stopper which is used in the lancet ejecting device. In the shown embodiment, a force is acting on the oblique portion of the stopper so that a distance between a rear end of the oblique portion and a rear end of a base portion is shorter.

FIG. 72 schematically shows a perspective view of the stopper when it is seen from its back side obliquely downward.

FIG. 73 schematically shows a perspective view of the stopper when it is seen from its front side obliquely downward.

FIG. 74 schematically shows a perspective view of the stopper when it is seen from its back side obliquely upward.

FIG. 75 schematically shows, in a perspective view, a manner in which a sensor element is placed on the lancet cassette according to the present invention wherein the sensor element is inserted between extensions and a concave portion on an upper half of the container.

FIG. 76 schematically shows a perspective view of a completed lancet cassette according to the present invention wherein the sensor element has been completely placed in the manner as shown in FIG. 75.

FIG. 77 schematically shows, in a perspective view, a manner in which the sensor element is placed on the lancet cassette according to the present invention wherein the sensor element is provided on a base surface of the upper half of the container.

FIG. 78 schematically shows, in a perspective view, a state wherein the sensor element has been placed in the manner as shown in FIG. 77.

FIG. 79 schematically shows, in a perspective view, a state wherein after the sensor element has been placed, the sensor element is fixed by fusing protruding portions followed by their solidification.

FIG. 80 schematically shows, in an elevation view, a relationship between moving directions of the ejecting member and the trigger member in the case where the ejecting member is entirely straight.

FIG. 81 schematically shows, in an elevation view, a relationship between moving directions of the ejecting member and the trigger member in the case where the front portion of the ejecting member is able to be bent.

FIG. 82 schematically shows, in an elevation view, a relationship between moving directions of the ejecting member and the trigger member in the case where the ejecting member is entirely straight.

FIG. 83 schematically shows a perspective view of the lancet for the purpose of explaining another embodiment of a weakened portion.

In the drawings, the reference numbers indicate the following elements:
100 . . . lancet, 102 . . . container, 104 . . . lancet cassette,
106 . . . pricking member,
107 . . . front end portion of lancet body, 108 . . . lancet body,
109 . . . a portion of pricking member, 110 . . . shield member,
111 . . . gap, 112 and 113 . . . weakened portion, 114 . . . space,
115 . . . reinforcing portion,
116 . . . tip portion of pricking member, 118 . . . sensor element,
119 . . . terminal, 120 . . . front end of sensor element,
121 . . . opening, 122 . . . cut away portion,
124 . . . point at which moving direction of pricking member intersects with extending plane of sensor element,
130 . . . plane or extending plane of sensor element,
132 . . . upper main surface, 134 . . . lower main surface,
136 . . . horizontal plane,
138 . . . point at which moving direction of pricking member intersects with extending plane of sensor element,
140 . . . blood intake port, 142 . . . lancet rear end,
144 . . . lancet hook portion,
146 . . . lancet pullout preventing portion,
148 . . . retracting portion, 150 . . . tab portion,
152 . . . cap portion, 154 . . . protruding portion,
160 . . . groove portion, 201 . . . inner surface,
202 . . . upper half, 203 . . . collision portion,
204 . . . lower half, 205 . . . collision portion,
206 . . . container front end, 207 . . . outer surface,
208 . . . opening, 210 . . . rear end, 211 . . . abutment portion,
212 . . . opening, 214 . . . convex portion, 215 . . . inner surface,
216 . . . concave portion, 218 . . . hinge portion,
220 . . . container preform, 222 . . . space,
224 . . . recess portion, 230 . . . protruding portion,
232 . . . concave portion, 360 . . . lancet ejecting member,
362 . . . leading end, 363 . . . pressing portion,
364 . . . protruding portion, 367 . . . side protruding portion,
368 . . . edge portion, 369 . . . a portion of ejecting member,
370 . . . trigger member, 370 . . . leading end, 372 . . . stopper,
373 . . . ejection spring, 374 . . . connector,
375 . . . return spring, 376 . . . return spring,
377 . . . cover member, 378 . . . flange portion,
379 . . . protrusion, 380 . . . rear end, 381 . . . protrusion,
382 . . . opening, 384 . . . partition member,
386 . . . flange member, 388 . . . flange member,
390 . . . channel portion, 392 . . . wall, 394 . . . guide portion, 396 . . . slit, 398 . . . guide, 399 . . . slit,
400 . . . lancet ejecting device, 500 . . . oblique portion,
502 . . . rear end, 510 . . . engaging portion,
512 . . . elastic member, 520 . . . front end,
522 . . . opening, 524 . . . rear end, 526 . . . opening,
528 . . . stopper portion, 530 . . . opening,
532 . . . locating member, 534 . . . convex portion,
536 . . . chamfered shape, 540 . . . front end, 542 . . . opening,
550 . . . guide portion, 560 . . . base portion, 561 . . . bottom,
562 . . . oblique portion, 564 . . . front end, 568 . . . rear end,
570 . . . rear end, 573 . . . inclined plane,
600 . . . electronic device, 602 . . . electronic device plate,
620 . . . upper surface, 622 . . . post, 624 . . . hole,
700 . . . housing, 702 . . . housing upper half,
704 . . . housing lower half,
706 engaging portion holding member,
710 . . . discharge button, 712 . . . elastic member,
714 . . . pressing member, 716 . . . leg portion, 718 slit,
719 . . . downward protruding member, 720 . . . opening,
722 . . . opening, 724 . . . leg portion, 726 . . . inclined surface,
728 . . . inclined surface, 730 . . . spring portion,
750 . . . concave portion, 752 . . . base surface,
754 . . . side surface, 758 . . . extension.

### DETAILED DESCRIPTION OF THE INVENTION

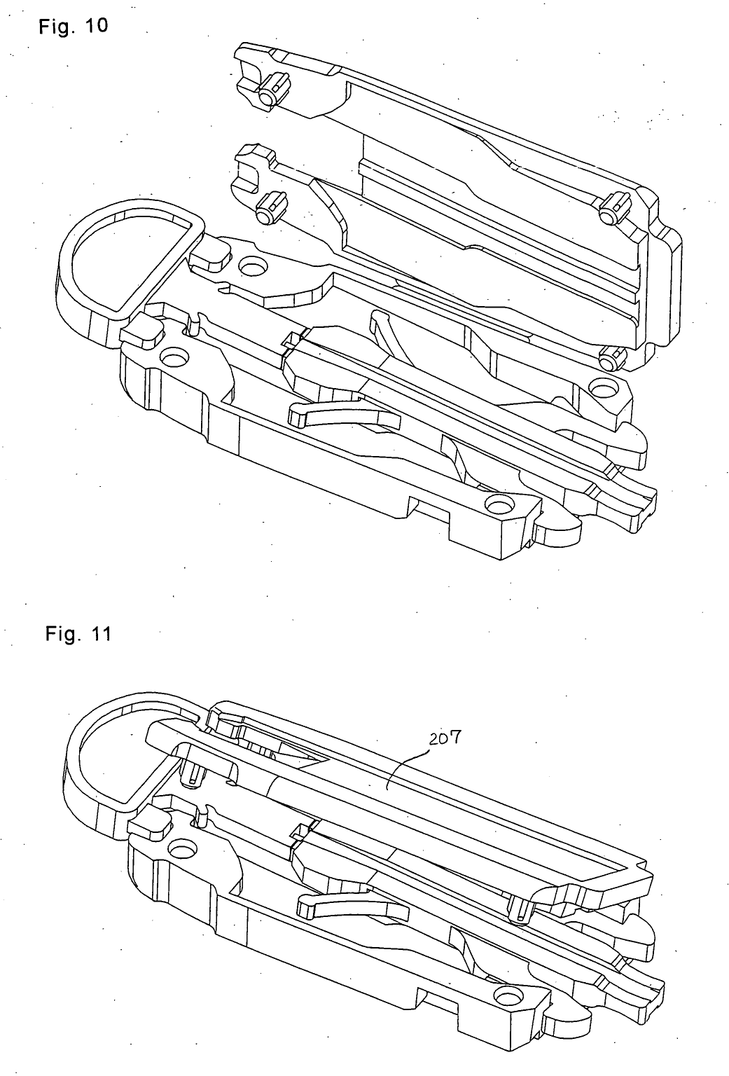

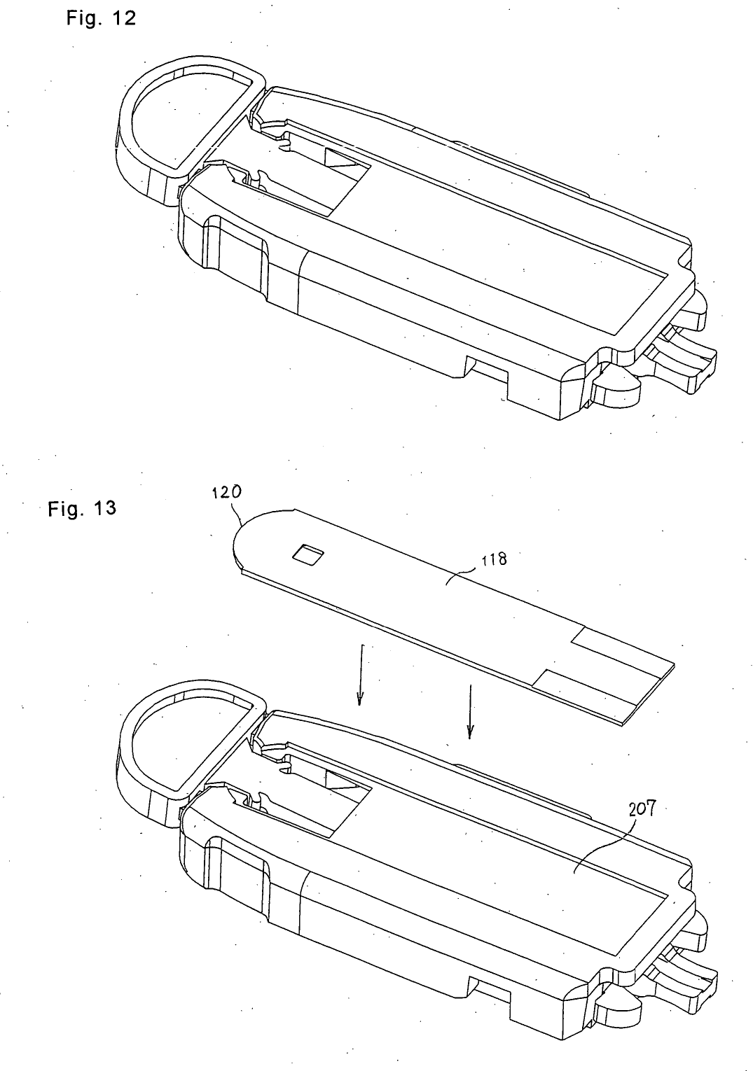

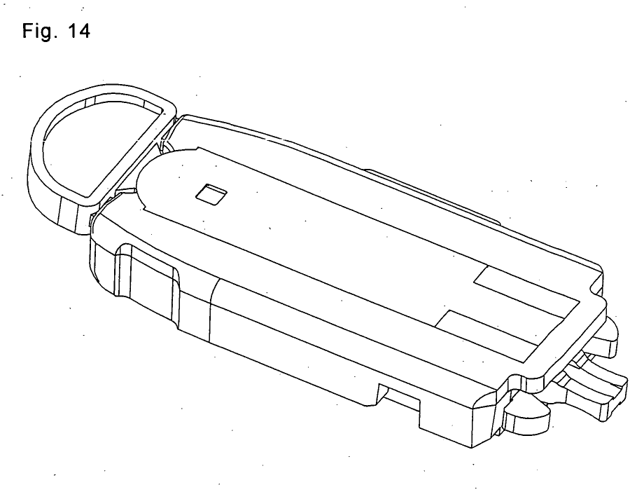

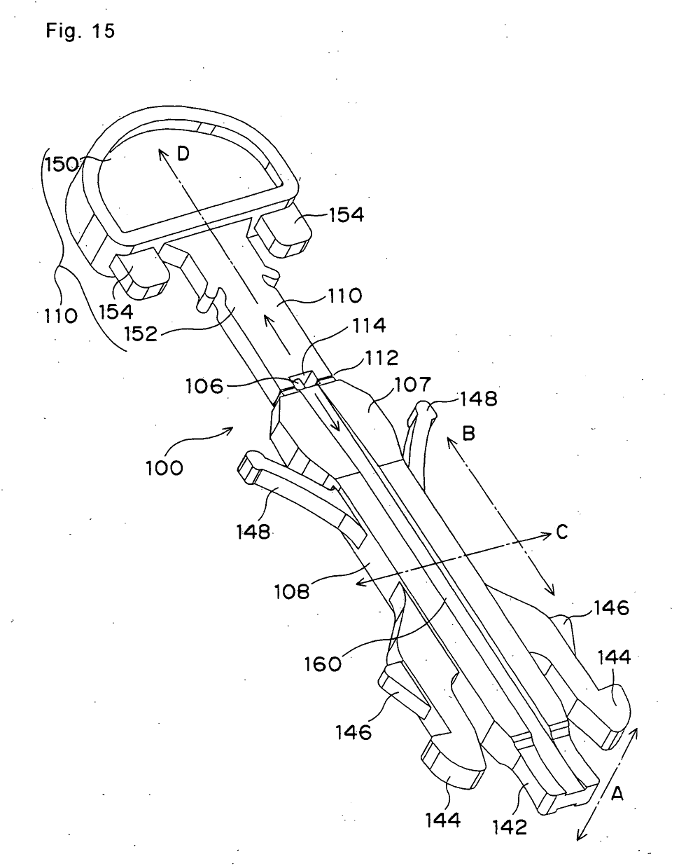

In the first aspect, the present invention provides a lancet cassette 104 (which is also referred to as merely a “cassette”) comprising a lancet 100 which comprises a pricking member (which may in the form of for example a needle or a blade, and preferably the needle) as well as a container 102 for the lancet. The lancet is substantially composed of the pricking member 106, a lancet body 108 and a shield member 110. The lancet body 108 and the shield member 110 are connected together through a weakened portion 112 in the lancet which has not been used. The pricking member 106 is enclosed with the lancet body 108 and the shield member 110. It is noted that the lancet is preferably produced by injection molding while the pricking member like a needle is inserted as described below, and in such case, a portion of the pricking member may be exposed because of the production convenience. For example, a portion of the pricking member is exposed in a space 114 in FIG. 15.

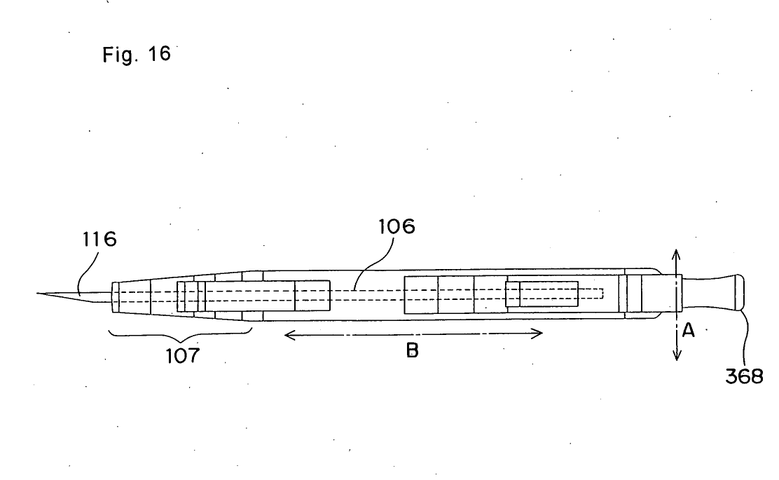

For the purpose of pricking, when a force is applied so as to separate off the shield member from the lancet body (as shown for example with the arrows in FIG. 15), the weakened portion 112 is broken. When the shield member 110 is pulled away from the lancet body 108, a tip portion 116 of the pricking member 106 is exposed from a front end of the lancet body 108 as shown in FIG. 16.

The container defines a space wherein the lancet moves while its tip portion is exposed so as to prick a predetermined portion. That is, the lancet moves in the space toward the predetermined portion with the tip portion of the pricking member being exposed, followed by pricking the predetermined portion and then the exposed pricking member is hold within the space of the container.

A sensor element 118 can be mounted on such container, which element is able to carry out a predetermined measurement (for example, blood glucose level measurement) as to blood which bleeds from the pricked portion. The sensor element is of a thin type as shown in the drawings, and it is usually in the form of a strip (namely, in an elongated sheet form). Thus described type of sensor element includes a blood intake port at or in the vicinity of its front end 120. Upon usual measurement, when an amount of spilt blood is attached to the intake port, the blood moves into the inside of the sensor element such that a predetermined measurement can be carried out. In an area where the predetermined measurement is carried out, two opposing electrodes are located, and a predetermined agent thin layer is placed between the electrodes. The predetermined measurement is carried out by measuring electric signals generated by a predetermined reaction which occurs in the predetermined agent thin layer which a body fluid reaches from the intake port by means of for example the capillary phenomenon. In order to transfer such electric signals to an electronic device which will be explained later, the sensor element comprises terminals usually at or in the vicinity of its rear end which are electrically connected to the above described electrodes respectively.

As a concrete sensor element, a blood glucose level measuring sensor element may be exemplified such as Dia-Sensor (trade name) commercially available from Arkray or AutoDisc sensor (trade name) commercially available from Bayer. It is obvious for those skilled in the art that the above described blood glucose level sensor is merely an example, and sensors may be used which are for measurement items (such as a cholesterol level) other than the blood glucose level.

Various types of such sensor elements are commercially available, in which there is a device to absorb a body fluid to be measured from its front end to its thin layer portion of the agent. Usually, such sensor element can carry out the predetermined measurement by depositing the body fluid to be measured to its front end.

The above sensor element can be mounted on an outer surface or an inner surface of an upper wall of the container which defines the space where the cassette moves. Thus, in one preferable embodiment of the present invention, the cassette comprises a sensor element 118 which carries out a predetermined measurement as to blood, for example a so-called biosensor element.

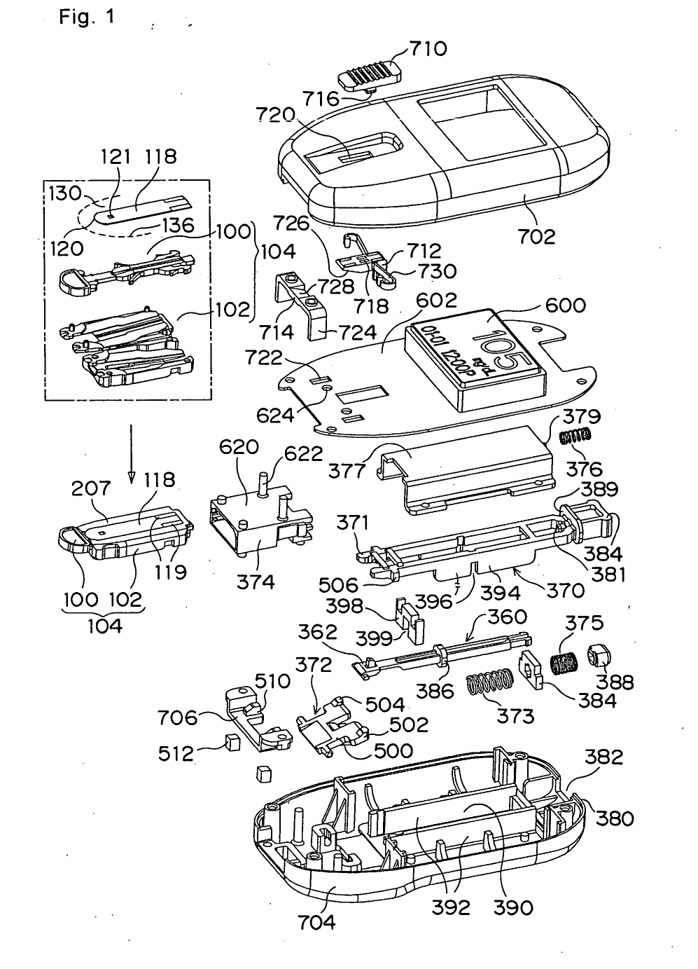

In one embodiment, the container 102 is comprised of an upper half 202 and a lower half 204, and the upper half forms the upper wall on which the sensor element can be mounted. That is, the sensor element is located on an outer surface or an inner surface of the upper wall. It is noted that in the embodiment shown in FIG. 1, the sensor element is placed on the outer surface 207 (see FIG. 11) of the upper half. Mounting of the sensor element may be carried out in any appropriate manner. For example, the sensor element may be attached by means of an adhesive. Alternatively, the sensor element may be fitted into the outer or inner surface of the half.

In the present specification, when the terms “up(per)” and “low(er)” and also the phrases related thereto which substantially include meanings of such terms (for example, “upward”, “upper side”, “downward” and “lower side”) as well as the terms “outer” and “inner” and also the phrases related thereto which substantially include meanings of such terms (for example, “outside” and “inside”) are used based on the cassette and the lancet ejecting device, which are in their assembled states.

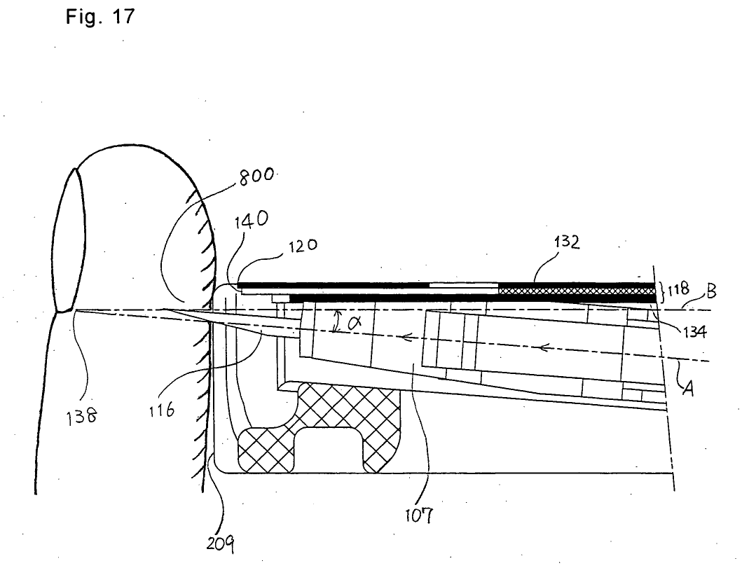

In one embodiment, the lancet cassette according to the present invention comprises the sensor element on its outer surface of its upper wall 207 and, the front end 120 of the sensor element is located close to the front end 206 of the container, preferably above its frontmost or its vicinity. In other preferable embodiment, the leading end 120 of the sensor element may be retracted a little from the frontmost 209 of the container as shown in FIG. 17, which is advantageous in that inhibition of pricking into the predetermined portion due to the pricking direction being inclined from the horizontal direction can be avoided.

Upon pricking the predetermined portion, when the front end 206 of the cassette is forced against the predetermined portion, a part of the predetermined portion enters somewhat the inside from the front end 206. Thus, there is substantially no difference between the case where the front end 120 of the sensor element is retracted a little from the frontmost of the cassette and the case where the front end 120 of the sensor element is located above the frontmost of the container.

When the sensor element is located as described above, a distance between an amount of bleeding blood and a sample intake port of the sensor element becomes short, so that an amount of the bleeding blood is likely to be in contact with the sample intake port. In other preferable embodiment, the sensor element is placed on the inner surface 201 of the upper half 202. In this case, the distance between an amount of bleeding blood and the sample intake port of the sensor element becomes shorter, so that an amount of the bleeding blood is more likely to be in contact with the sample intake port.

In other embodiment, the cassette does no have such sensor element, and in this case, a user mounts the sensor element onto the cassette before using the lancet for pricking, particularly on the outer surface or the inner surface of the upper half.

As to the cassette according to the present invention, a moving direction of the pricking member of the lancet intersects with the extending plane of the sensor element in the vicinity of the leading end of the sensor element. FIG. 17 schematically shows the lancet according to the present invention when viewing from its side. The “moving direction of the pricking member of the lancet” is a moving direction of the pricking member 106 in the cassette upon pricking (that is, the direction shown with the alternative long and short dash line having the arrows A), which substantially corresponds to an extending direction of the pricking member within the cassette.

The “extending plane of the sensor element” means a hypothetical flat surface which includes the main surface of the sensor element 118 which is of a flat thin type. Such flat surface 130 is schematically shown in FIG. 1. The flat surface defines an infinite spread, but a limited area thereof is shown with the broken line 136 so as to visibly understand. It is noted that the sensor element has its upper main surface 132 and lower main surface 134, and the extending plane of the sensor element may be either main surface since it is of a thin type. As another expression, a thickness of the sensor element may be neglected in the present invention, so that it is of substantially no problem to consider that the upper main surface corresponds to the lower main surface, which allows to render the extending plane to be unitary. Such extending plane is regarded to be the flat surface 130 which includes and spreads around the sensor element. It is noted that the extending plane 130 is shown with the alternative long and short dash line in FIG. 17 while shifted downward a little from its true position for the purpose of readily understanding.

Clearly seen from FIG. 17, the moving direction A of the pricking member is not parallel to but intersects with the extending plane 130 or B of the sensor element. The point 138 at which they thus intersect is located in the vicinity of the front end 120 of the sensor element. The “front end of the sensor element” means a frontmost end of the sensor element (that is, a frontmost end along the longitudinal direction of the sensor element), and usually the intake port 140 of the blood is located at the leading end. The “vicinity” means a potion at just the front end itself, in a portion in front of the front end or at the back of the front end. In the case of “at the back of the front end”, since the intersecting point 138 is located behind the front end 120, the tip portion 116 of the pricking member pushes up the portion of the sensor element at the back of its front end before pricking the predetermined portion. In such case, the front end portion of the sensor element is to be able to elastically bend a little when being pushed up by the pricking member.

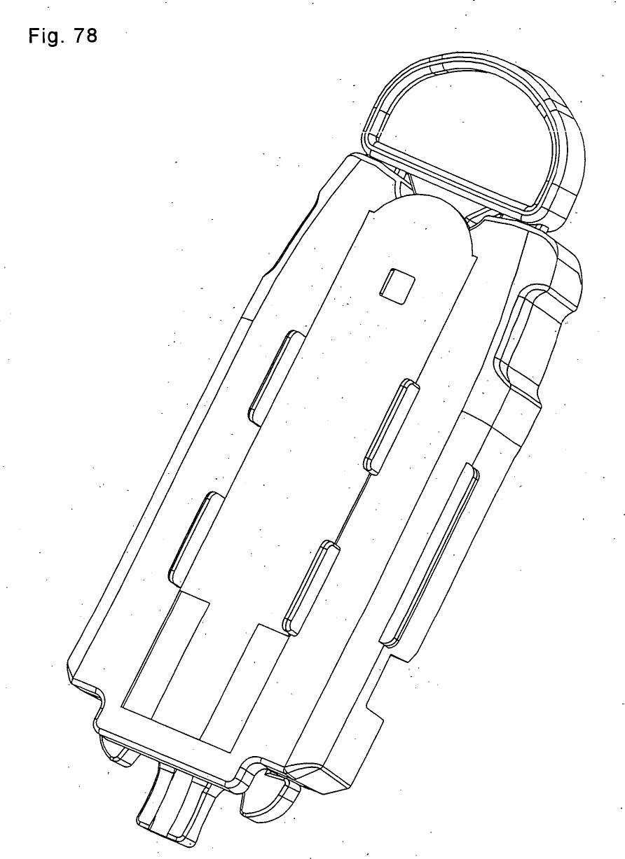

In other embodiment wherein a front end portion of the sensor element 118 set back in its center for example as shown in FIG. 77, that is, the front end portion has a cut away portion 122 so that the portion immediately behind the front end of the sensor element is not pushed up as described above, the intersecting with the moving direction of the pricking member happens in the cut away portion the intersecting point is shown with “124”, to which case the above mentioned “at the back of the leading end” is applicable. With the sensor element 118 of the embodiment shown in FIG. 77, the intersecting point 124 is located behind the front end 120. The shown sensor element 118 is of a thin layer type sensor element of which center portion in the front end is cut away to form a concave portion, and its blood intake port 140 is located at the innermost recess of the concave portion (i.e. at the bottom thereof), but it may be located at other portion.

Basically, the position of the intersecting point 138 may be shifted by changing an angle α formed by the pricking direction A and the extending plane B. Provided that the lancet and the container are not changed except the angle α, the intersecting point shifted backward when the angle is increased while shifted forward when decreased. When the pricking direction A and the extending plane B are parallel to each other (that is, the angle α is 0°), pricking is likely to happen at an angle of 90° relative to the predetermined portion but the distance between the front end of the sensor element and an amount of the bleeding blood is long. When the angle α is set greater than 0°, the pricking direction A intersects with the extending plane B.

The cassette according to one preferable embodiment of the present invention is configured such that an amount of the bleeding blood after pricking is able to be in contact with the front end of the sensor element, namely, the intake port, so that the blood is supplied to the inside of the sensor element so as to carry out the predetermined measurement without an additional operation after pricking. In other words, the “vicinity of the front end of the sensor element” in this embodiment means an area of the extending plane which allows an amount of the bleeding blood after pricking to be present at the intake port. Generally, the vicinity of the front end means an area of the extending plane located in the range from 3 mm behind the front end of the sensor element up to 10 mm ahead thereof, preferably in the range from 2 mm behind the front end of the sensor element up to 8 mm ahead thereof, and more preferably in the range from the position just at the front end of the sensor element up to 7 mm ahead thereof. When referring to such range, the moving direction of the pricking member is based on a center axis of the pricking member, and the extending plane is based on the lower surface of the sensor element.

The present invention, however, does not exclude an embodiment of the cassette wherein an amount of the bleeding blood is more or less away from the intake port, which requires an additional operation after pricking so as to make the amount of bleeding blood be in contact with the intake port, and this embodiment is less preferable. In such embodiment, the moving direction of the pricking member of the lancet intersects with the extending plane in an area except the vicinity of the front end of the sensor element, that is, intersects in an area which is separated forward by a distance of more than 10 mm from the front end of the sensor element. Such distance is preferably not longer than 20 mm and more preferably not longer than 15 mm. The embodiment which requires the additional operation upon pricking can be generally discriminated from the embodiment which requires no additional operation upon pricking by the area of the vicinity of the front end as described above. Such discrimination is a kind of measure but not absolute. Thus, even when the same cassette is used, the additional operation may be required or not required depending on for example an angle of the predetermined portion relative to the opening at the front end of the cassette when pricking, an amount of blood which bleeds out by pricking or the like.

Recently, a thickness of the pricking member tends to be smaller for the purpose of alleviating pain of users. Therefore, an amount of blood which bleeds by pricking has become smaller, so that it is not easy to identify the pricked position. Particularly, a diabetic patient sometimes has bad eyes, and it may be often not so easy to make an amount of blood be in contact with the front end of the sensor element. By using the cassette according to the present invention, no additional operation becomes required as described above, which is very convenient to the diabetic patient.

When the angle α is large which is formed by the moving direction of the pricking member and the extending plane of the sensor element, an incision may be larger since pricking is carried out at a substantially slant relative to a surface of the predetermined portion (for example, a skin surface of a finger tip). Therefore, an excessively large angle α is not preferable. In the present invention, the angle α is preferably in the range between 2° and 10°, more preferably in the range between 3° and 8°, most preferably in the range between 4° and 5°, and for example 45°. This angle means a smallest angle defined by a plane and a straight line which intersects with the plane (i.e. the pricking direction). When such angle is excessively large, the angle which is formed by the surface of the predetermined portion to be pricked and the pricking direction is excessively away from 90°, so that the incision formed in the predetermined portion by pricking may become larger, which may cause more pains. Also, a thickness of the container which will be described below becomes larger, a volume of a packaged cassette as an article is large, which is not preferable from viewpoints of for example packaging and transportation of the article. On the other hand, when the angle is excessively small to be close to 0°, the distance between the intake port of the sensor element and the pricking position becomes excessively long, so that it is possible that an amount of bleeding blood cannot be in contact with the intake port of the sensor element after pricking.

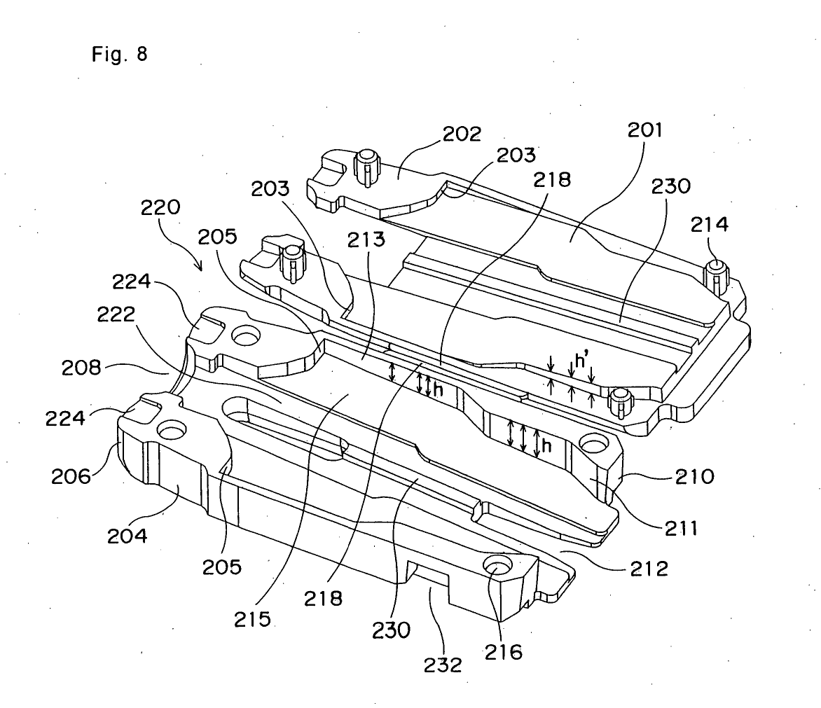

In order that the lancet is moved obliquely upward as described above, the container is designed so that an inner surface of the lower half 204 of the container guides the lancet forward and obliquely upward. Since the inner surface 215 of the lower half of the container in fact supports the lancet, the lower half is designed such that the inner surface 215 of the lower half of the container entirely extends forward and obliquely upward. In the embodiment shown in FIG. 8, it is seen that a height “h” of a wall 213 which defines a side of the lower half 204 (shown with the arrows having various heights) is lower toward the front of the container, so that the inner surface of the lower half extends forward and obliquely upward, which allows the lancet to move forward and obliquely upward in the container. Also, in the embodiment shown in FIG. 8, it is seen an inner surface 201 of an upper half 202 (which inner surface defines a ceiling of an inner space of the container when assembled into the container) extends forward and obliquely downward (see h′), so that the ceiling extends forward and obliquely upward when assembled into the container.

Generally, it is preferable to design such that the inner surface of the upper half and the inner surface of the lower half are substantially parallel to each other. Therefore, when the container which houses the lancet is viewed from its side, the flat surface of the container (i.e. the outer surfaces of the upper half and the lower half) extends horizontally while the lancet is contained in the container such that it extends forward and obliquely upward relative to the horizontally extending surface.

The lancet according to the present invention is substantially flat as a whole as seen from FIGS. 15 and 16, that is, its thickness dimension (the dimension shown with the arrow A in FIG. 16) is relatively smaller than the other dimension(s) (for example, the length (the dimension shown with the arrow B in FIG. 16) and the width (the dimension shown with the arrow C in FIG. 16)). Various portions which constitute the lancet are extending substantially perpendicularly relative to the thickness direction of the lancet, that is, they are present on a flat surface. Those various portions of the lancet are substantially line-symmetric with respect to the pricking direction (the direction shown with the arrow D in FIG. 15). Generally, various members which constitute the container and the lancet ejecting device are also substantially line-symmetric with respect to the pricking direction similarly to the lancet. It is noted that although the pricking direction is at a slant relative to the extending plane as the horizontal plane as described above, the term “symmetric” is used in the specification with neglecting such slant. Neglecting the slant is in fact of no problem as far as the “symmetric” is concerned, and also convenient for understanding the invention.

The lancet 100 is substantially comprised of the pricking member 106, the lancet body 108 and the shield member 110, and the pricking member 106 is in the lancet body 108 and the shield member 110. In a state before using of the lancet, most of the lancet body 108 except a portion thereof is contained in the container 102 as described below. The shield member 110 comprises a tab portion 150 and a cap portion 152, which is contained in the container. The cap portion 152 covers the tip portion 116 of the pricking member while being present around the tip portion 116, so that the tip portion is shielded (or sealed or blocked) against its ambient environment. The tab portion 150 facilitates a user pinching and pulling off the shield member 110 so as to separate the shield member 110 from the lancet body 108.

The container 102 is formed by connecting the upper half 202 and the lower half 204 are connected while they are opposed as described above, and the above explained lancet 100 is housed in the space defined by the container while it is able to be ejected. The container 102 includes an opening 208 at its front end 206 and an opening 212 at its rear end 210, and it is formed by connecting the upper half and the lower half together while they are opposed. The connection may be carried out in any appropriate manner. For example, an adhesive or a screw may be used. In a particularly preferable embodiment, the upper half and the lower half are connected by means of press fitting. For example in FIG. 8, convex portions 214 and concave portions 216 are provided on the upper half 202 and the lower half respectively for the purpose of the press fitting. Such container is thicker than the lancet a little, but it is flat as a whole similarly to the lancet, which is seen from for example from FIG. 16.

From a viewpoint of production convenience, it is preferable to produce a container preform 220 in which the upper half 202 and the lower half 204 are connected together by means of a hinge portion 218 portions located between one sides thereof. The preform may be produced by for example injection molding. The upper half and the lower half are connected together by folding the preform around the hinge portion. For such connection, it is preferable that posts (or small columns) 214 and holes 216 into which the posts are press fitted are provided to the halves respectively.

The lancet having the pricking member of which tip portion 116 is exposed is ejected by means of the trigger member as explained below. Once ejected, the lancet is able to move forward and backward before and after pricking the predetermined portion in the space 222 defined by the upper half and the lower half, but it is substantially not able to move toward other directions. Thereby, the lancet is surely ejected as predetermined. It is noted that prior to starting the ejection, the lancet is engage with the container so that movement of the lancet is limited and it is substantially not able to move as described below.

As to the lancet 100 which is able to move as explained above, the pricking member 106 (for example, a needle or a blade, and preferably the needle) is enclosed by the lancet body 108 and the shield member 110 in its unused state. Such lancet is preferably produced by insert molding of a resin (for example, a polyethylene resin (such as HDPE, LDPE and the like), a polypropylene resin, a nylon resin, a polyacetal resin, a polystyrene resin, and various copolymers of monomers for the above mentioned resins) so as to integrate the lancet body 108 and the shield member 110. Upon the insert molding, a border portion between the lancet body 108 and the shield member 110 may be made to have a small thickness around the pricking member 106 so as to form a notch portion which corresponds to a weakened portion 112.

It is preferable that weakened portions 112 are produced while exposing a portion of the pricking member 106 around its whole periphery between the lancet body 108 and the shield member 110 by forming resin portions on either side of the exposed portion in which resin portions notches are provided espectively. That is, the resin portions are formed to define a space 114 in which the portion of the pricking member 106 is exposed. When said portion of the pricking member is not exposed, a force required to break the weakened potion become excessively large.

Such notches are formed by inserting opposing blades vertically into the resin portion at predetermined positions thereof on either side of the exposed pricking member as if the blades sandwiched the resin portion. For example, the blades (such as razors) are inserted into the resin portion from its top and bottom (or its left side and right side) respectively so as to form the notches. It should be noted that some resin portion has to be remain intact such that pulling off of the shield is possible as described later. Generally, the notches are formed such that a thickness of the resin portion between the bottom of one notch and the bottom of the other opposing notch is preferably 0.02 mm to 0.2 mm, for example about 0.06 mm.

In an embodiment other than the embodiment wherein the notches are provided on the both sides of the exposed pricking member portion, the weakened portion which separates the lancet 100 into the lancet shield member 110 and the lancet body 108 is formed of a resin in the form of a thin layer which is located around a portion of the pricking member which connects the lancet shield member 110 and the lancet body 108. Such thin resin layer surrounds the pricking member portion and it is preferably comprises reinforcing portions in the form of wings which extend horizontally and vertically relative to the extending direction of the pricking member (i.e. a direction parallel to the direction shown with the arrow C in FIG. 15 in an area where the lancet shield member 110 and the lancet body 108 are adjacently present. With only the thin layer around the pricking member, the layer may be broken by a force accidentally applied thereto, and the reinforcing portions are effective for preventing such breakage. Thicknesses of the thin layer around the pricking member and also of the reinforcing portions are from 0.02 mm to 0.2 mm and for example about 0.06 mm.

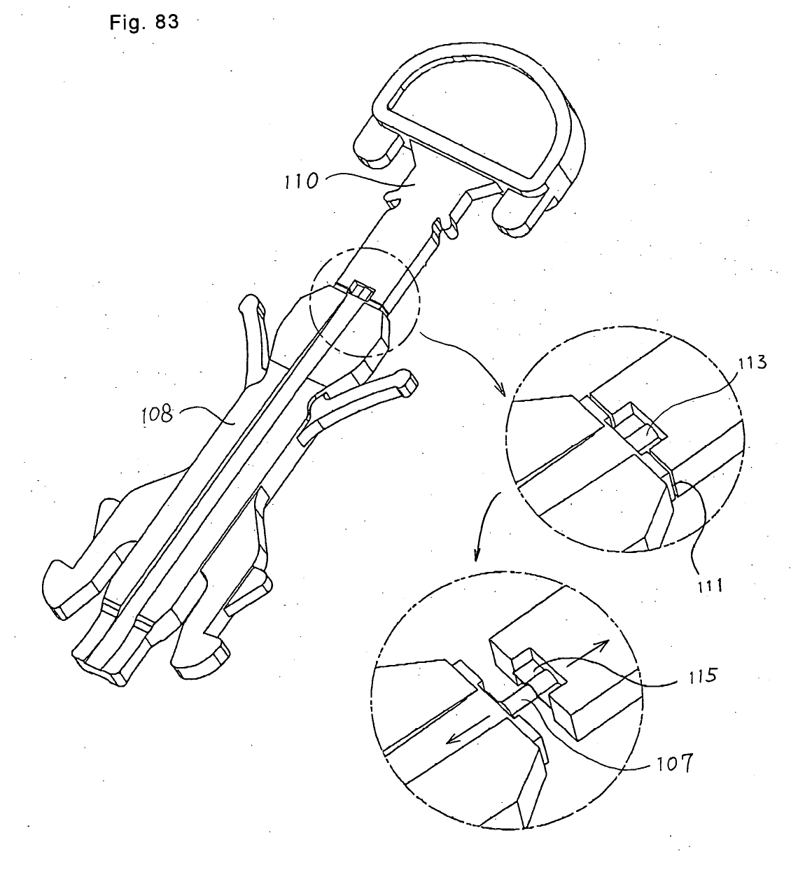

The above described weakened portion is illustrated in FIG. 83. In the shown embodiment, the weakened portion 113 is in the form of a thin layer resin which surrounds a portion 109 of the pricking member, and it includes reinforcing portions 115 in the form of wings which extend horizontally from the both sides of the weakened portion 113. End surfaces of main portions of the lancet shield member 110 and the lancet body 108 are spaced so as to form a gap 111 between them before using of the lancet. In FIG. 83, a connecting portion between the lancet shield member 110 and the lancet body 108 (i.e. an area enclosed with the chain double-dashed line) is shown enlarged. The upper enlarged view schematically shows a state before breaking the weakened portion 113 while the lower enlarged view schematically shows after the weakened portion 113 has been broken by application of a force as shown with the arrows, so that a portion 109 of the pricking member is exposed. In the lancet which is explained above with reference to FIG. 15, by surrounding the pricking member portion which is exposed in the space 114 located between the weakened portions 112 by a thin resin layer, and not connecting the lancet shield member 110 and the lancet body 108 through the notches while they are originally separated from each other, another embodiment of the weakened portion is provided. Such embodiment is preferable since the weakened portion is produced simultaneously with molding of the lancet 100 and the pricking member 106 is entirely enclosed by the resin.

The lancet body 108 further comprises a lancet rear end 142, lancet hook portions 144 on either side of the lancet rear end and lancet pullout preventing portions 146 each located in front of the lancet hook portion. Before using the lancet, the lancet rear end 142 protrudes outward from the opening 212 of the rear end 210 of the container 102, and also the lancet hook portions 144 protrude outward from the opening 212 of the rear end 210 of the container 102 and are adjacent to and engaged with the outside of the rear end 210 of the container. The lancet pullout preventing portions 146 are located in the space 222 within the container while being adjacent to or near the inside of the rear end 210 of the container 102.

After the ejection of the lancet, the lancet rear end 142 and the lancet hook portions 144 are located in the space 222 of the container. The lancet body 108 further comprises retracting portions 148 which moves the lancet backward which has moved forward in the container and pricked as described below. Various portions (142, 144, 146 and 148) which the lancet includes are preferably formed integrally with the lancet body 108. For example, the above mentioned insert molding can produce the lancet body which is integrated with the various portions as well as which is simultaneously integrated with the shield member.

It is noted that the above described container 102 in which the lancet 100 is housed is called as the “cassette” regardless of whether it is in the state after or before the lancet has been used. As shown in FIG. 15, the tab portion 150 may have protruding portions 154 which are located in recesses 224 formed at the front end 206 of the container between the upper half 202 and the lower half 204. The provision of the protruding portions 154 in the recesses 224 limits the movement of the shield member 110 even when an accidental force is applied to the tab portion 150 as to the cassette before its using, so that breakage of the weakened portion 112 due to the accidental force is prevented.

In the second aspect, the present invention provides a lancet assembly which is comprised of the lancet cassette comprised of the above described lancet and container which houses the lancet, and the lancet ejecting device, and such assembly ejects the lancet so as to prick the predetermined portion.

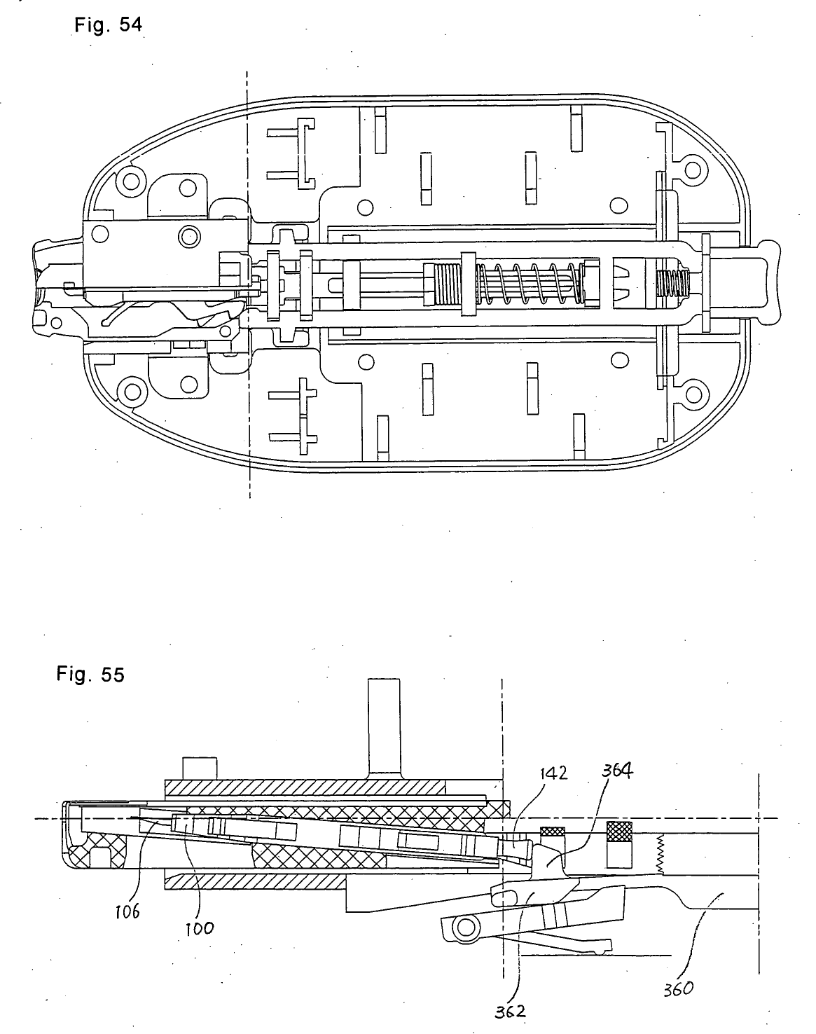

In the specification, the term “ejection (or eject)” as to the lancet means that the lancet 100 moves with dashing forward by means of being instantaneously pushed forward of the rear end of the lancet (by a force applied by a spring means as explained below) via the lancet ejecting member 360 (which is also referred to as merely an ejecting member), particularly its leading end 362, and more particularly a protruding portion 364 provided to the leading end while the rear end abuts against the ejecting member (see FIGS. 54 and 55). Such ejection may be likened to release of an arrow using a bow. That is, the ejecting member corresponds to a bowstring, and the lancet corresponds to the arrow. As readily understood, the abutting relationship between the lancet rear end and the ejecting member ends during the ejecting process (i.e. on the way of ejecting), and preferably a little before, after or substantially just when the tip portion of the pricking member pricks the predetermined portion.

That is, the relationship is discontinued in any one of the following: (a) in the case when the lancet rear end 142 disengages from the ejecting member 360, particularly its protruding portion 364 so that only the lancet 100 moves forward and protrudes from the opening at the front end of the container so as to prick the predetermined portion; (b) in the case when the lancet 100 moves forward while the lancet rear end 142 is abutting against the ejecting member 360, particularly its protruding portion 364 so that it protrudes from the opening at the front end of the container so as to prick the predetermined portion, and then the lancet rear end 142 disengages from the ejecting member 360, particularly its protruding portion 364 during backward movement of the lancet; and (c) in the case when the lancet 100 moves forward while the lancet rear end 142 is abutting against the ejecting member 360, particularly its protruding portion 364 so that it protrudes from the opening at the front end of the container so as to prick the predetermined portion and simultaneously with such pricking, the lancet rear end 142 disengages from the ejecting member 360, particularly its protruding portion 364. Since the movement of the lancet upon ejecting is extremely rapid, it is substantially impossible to distinguish the above three cases with eyes.

In the case of (a), momentum of the moving lancet may be not completely sufficient for pricking. In the case of (b), the force which moves the ejecting member forward may be applied to the predetermined portion upon pricking, so that a pain upon pricking may increase. Considering these, the case (c) or a case (a′) wherein just prior to pricking, the rear end 142 of the lancet disengages from the ejecting member 360 in the case of (a) is the most preferable.

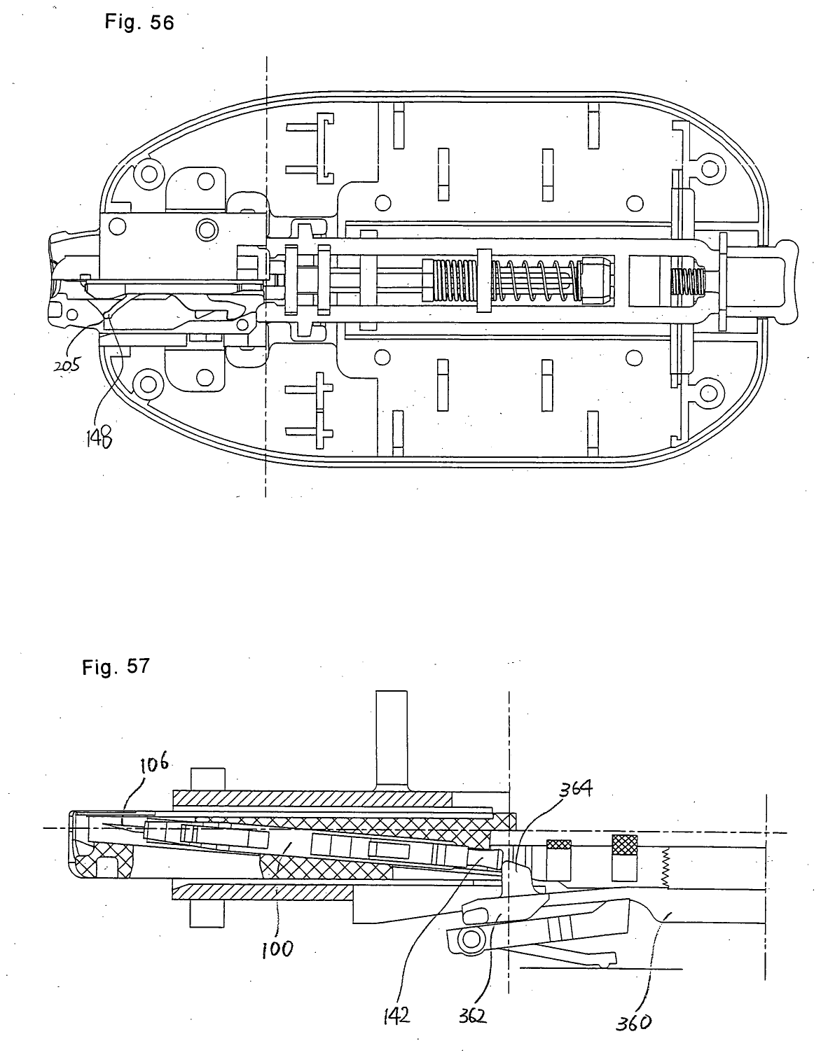

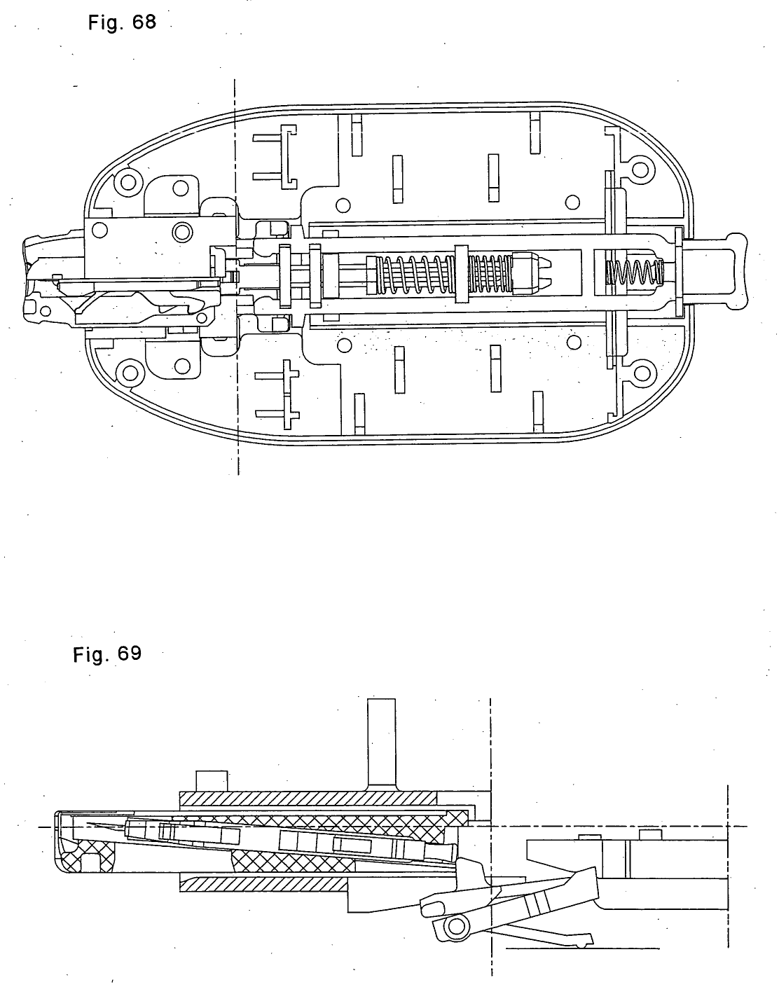

In order to make the case (c) or (a′) possible, an upper edge portion 366 (see FIG. 32) of the protruding portion 364 of the ejecting member as well as a lower edge portion 368 of the lancet rear end has a round shape as shown in FIGS. 56 and 57. Just prior to disengaging of the lancet rear end 142 from the ejecting member 360, particular its protruding portion 364, they are configured such that only those edge portions are in contact with each other as shown in FIG. 59. After pricking the predetermined portion, the lancet 100 moves backward by means of its retracting portions 142, and the exposed tip portion 106 of the pricking member is contained in the container 102. The ejecting member 360 moves forward a little after disengaging from the lancet rear end 142 as described later, but then an action of a return spring 375 moved backward and stops it. In thus stopped state, the protruding portion 364 is located obliquely downward as to the lancet rear end 142 as seen from FIG. 69. The ejecting mechanism of the lancet as described above is advantageous as to that the lancet holder which is required in the prior art lancet ejecting device is unnecessary; that energy required for pricking is surely transferred to the lancet from the ejecting member 360, and that even when a used lancet is tried to be used by charging the lancet ejecting device with it, pricking is impossible since the state shown in FIGS. 68 and 69 is reached wherein a so-called coking operation is impossible (that is, it is impossible to move the ejecting member back).

In the present invention, the lancet body 108 comprises the retracting portions 148 as described above. The retracting portions 148 protrude obliquely forward from the both sides of the lancet body 108. They are able to elastically deform, and move forward along the pricking direction such that they, preferably their leading end portions hit against collision portions (203 and/or 205) provided on both sides of and inside the upper half and/or the lower half just prior to, substantially simultaneously with or just after pricking the predetermined portion, followed by being deformed, preferably bent.

The above described retracting portion has a function to move the lancet backward after the lancet ejected forward has pricked the predetermined portion. When the lancet intensely moves along the pricking direction, the retracting portions hit against the collision portions (203 and/or 205) and deform due to the energy upon hitting of the lancet so as to stop the forward movement of the lancet. Immediately thereafter, the retracting portions tend to return to their original forms so as to move the lancet back so that the tip portion of the pricking member is pulled out of the pricked portion and retracted into the container. It is noted that the stopping of the forward movement of the lancet and moving backward of the lancet are helped by hitting against and pricking the predetermined portion of the pricking member.

The retracting portions in one embodiment are elongated and in the form of wings which extend obliquely forward from the both sides of the lancet body as shown in FIG. 8. Such wings hit against walls (which function as the collision portions) at the front end of the so that they elastically bend so as to store kinetic energy of the being ejecting lancet, and retract the lancet by means of forces of the wings which are to return to their original forms. In order to make thus elastic deformation possible, it is preferable that the lancet except the pricking member 106 is made of a resin, particularly a resin which is rich in flexibility and that a whole of the lancet is formed integrally so as to enclose the pricking member. It is preferable that the lancet is formed by injection molding, and particularly insert molding such that it encloses the pricking member.

After pricking, when the lancet is moved back as described above, the lancet hook portions 144 hit against walls as the abutment portions 211 inside the rear end of the container, so that the lancet cannot further be moved backward. Also, when the lancet tries to move forward in the container, it cannot substantially move forward since the retracting portions 148 of the lancet 108 abut against the collision portions 203 and/or 205. Therefore, the tip portion 116 of the pricking member 106 does not protrude outward from the opening 208 at the front end 206 of the container 102. As a result, the rear end 142 of the lancet cannot protrude outward from the opening 212 of the rear end of the container. Therefore, after the ejection, the lancet body 108 having the pricking member 106 of which tip portion 116 is extruding is in the state wherein the whole of the lancet body is substantially contained in the container 102. Prior to using the lancet (that is, before the ejection), the lancet rear end 142 and the lancet hook portions 144 have been exposed outside from the opening 212 of the rear end of the container 102, but they have not been exposed after the ejection, which differentiates the post-ejection and the pre-ejection. Thus, whether a lancet has been ejected (thus, whether it has been used) or not is readily seen by confirming with eyes whether or not the lancet rear end 142 and the lancet hook portions 144 are located outside the container.

It is configured that in the state wherein the lancet body 108 is contained in the container 102 after the ejection, even when any force which tries to move the lancet body 108 backward is applied accidentally, the lancet hook portions 144 abut against the abutment portions 211 inside and at the rear end of the container, so that the lancet cannot be moved backward any more. For the purpose of such configuration, an angle of the abutment portions 211 relative to the pricking direction is large as shown (for example 30°, 45° or the like), i.e. the angle is to be nearer to 90°.

When the lancet is used, the shield member 110 which is integrated with the lancet body 108 is to be separated from the lancet body 108 as shown in FIG. 15. The separation is achieved by the breakage of the resin at the weakened portion 112 which connects the lancet body 108 and the shield member 110 by applying a force which pulls away the shield member 110 from the lancet body 108, for example by applying a force which pulls off the tab portion 150 of the shield member 110 protruding from the opening 208 at the front end 206 of the container 102 while holding the container 102 which includes the lancet 100. By such separation, the tip portion 116 of the pricking member 106 is protruding forward at the front end of the lancet body 108. In other embodiment, the breakage of the weakened portion 112 may be carried out by applying a force so as to pull out the tab portion 150 from the lancet ejecting device after engaging with the device the cassette which contains unused lancet of which shield member and the lancet body are integrated.

When the force is applied for the separation, such force to pull out is effectively used to facilitate the separation since the lancet hook portions 144 are engaged with the outside of the container rear ends 210. Further, the hook portions 144 remain engaging with the rear end 210 so that the protruding tip portion 116 does not protrude outside from the container 102 and kept retracted from the opening 208 at the front end 206 as shown in FIG. 19.

The lancet has to move forward smoothly in the space of the container in the course of being ejected. For that purpose, the lancet has a guide groove 160 on its top side and/or bottom side which is parallel to the pricking member (and therefore, parallel to the pricking direction D), while the inner surface on the upper half 202 and/or the lower half 204 of the container has a protruding portion 230 which can fit into and smoothly move in the groove (and which is preferably complementary to the groove). Such relationship between the groove and the protruding portion fitted therein may be vice versa, that is, the hal(ves) has the groove(e) while the lancet has the protruding portion(s). The protruding portion is preferably elongate as shown in FIG. 8, which ensures more straight forward movement of the lancet.

The cassette above described is fitted into the lancet ejecting device (which is hereinafter also referred to as merely an “ejecting deice”), which leads to the lancet assembly according to the present invention. Using such lancet assembly, the pricking member can prick the predetermined portion. Thus, the cassette can have a function to be fitted into the ejecting device and to eject the lancet contained in the cassette.

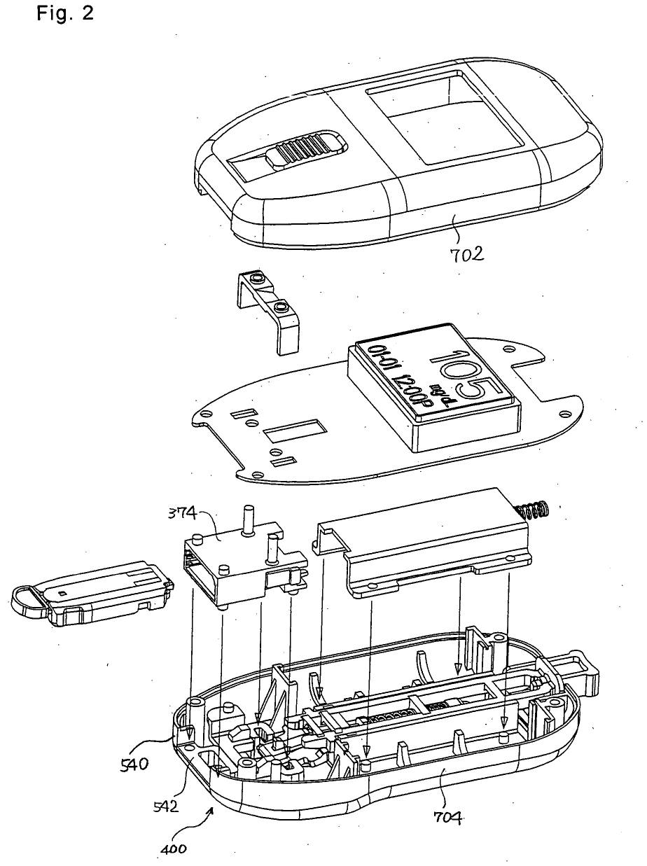

The ejecting device 400 comprises the lancet ejecting member 360, a trigger member 370, a stopper 372 and a connector 374, which are contained in a housing 700 (see FIG. 2). The ejecting member 360 has a function to eject the lancet 100 contained in the cassette 104 so that the pricking member pricks the predetermined portion. The trigger member 370 has a function to initiate the ejection by means of the ejecting member 360 by moving forward with a forward force applied from the outside, for example a pressing force by means a finger tip, so as to release the ejecting member 360 from its constraint state while a forward force is being applied to the ejecting member. The ejecting member is moved back to a predetermined position (i.e. its position before the cassette has not been fitted into) after moving forward for the ejection. Thereby, the ejecting member becomes in such condition that when a next cassette which contains unused lancet is charged, the rear end of the lancet can abut against the leading end of the ejecting member. Further, the trigger member 370 is configured such that when the force which is applied to the trigger member is released, the trigger member moves backward to its original position.

In order to apply the forward force to the ejecting member as described above, an ejection spring 373 is provided, and also in order to apply a backward force to move the ejecting member 360 backward, a return spring 375 is provided, and both of the springs are provided so as to be associated with the ejecting member 360. Further, another return spring 376 is provided while associated with the trigger member 370 which spring applies a backward force so as to return the trigger member 376 to its original position.

The return spring 376 is arranged so as to apply the backward force to the trigger member 370 when no force is applied between the housing and the trigger member. As a result, a flange portion 378 which is located at the rear end of the trigger member 370 is kept in a bias condition by the return spring 376 wherein it is in contact with the inside around an opening 382 at the rear end 380 of the housing. When a force which tries to move the rear end 384 of the trigger member 370 forward is applied to the trigger member (for example, when the rear end 384 is pressed forward by a finger tip), the return spring 376 is compressed, and the trigger member is able to move forward provided that nothing prevents the movement. When such force is released (for example, when the finger tip which is being pressing is taken away), the trigger member 370 moves backward since the return spring 376 tends to return to its original form, and the flange portion 378 abuts against the inside of the opening periphery of the housing 380.

It is preferable that the return spring 376 is located between the inside of the flange portion 378 of the trigger member and the rear end of 379 of a cover member 377 (or a protrusion provided thereon) which is fixed to the housing so as to cover the trigger member 370, so that when a forward force is applied to so as to move forward the trigger member 370 relative to the housing, the return spring 376 is compressed, and on the other hand when such a force is released, the trigger member 370 moves backward since the return spring 376 tends to return to its original form.

The ejecting member 360 comprises the ejecting spring 373 which is located in front of a partition member 384 fixed to the trigger member 370 as well as the return spring 375 behind the partition member 384. The ejection spring 373 is placed in its compressed condition (from its expanded condition when no force is applied) between the partition member 384 and a flange portion 386 located in a front portion of the ejecting member. The return spring 375 is placed in its compressed condition (from its expanded condition when no force is applied) between the partition member 384 and a flange member 388 located in a rear portion of the ejecting member. When the lancet rear end 142 is abutting against the protruding portion 364 at the leading end portion of the ejecting member 360 and moves the ejecting member backward, the trigger member 370 cannot moves forward due to a function of a stopper which will be described later, and it is kept substantially unmoved since the flange portion 378 is abutting against the rear end of the housing as described above. The partition member 386 is kept in an unmoved state as to the trigger member 370 as described later.

Therefore, in the state wherein the two springs 373 and 375 are placed as described above, when the ejecting member 360 is moved backward by means of the lancet rear end 142, the flange portion 386 moves backward, but the partition member 384 does not move backward, so that the spring 373 is further compressed (the primary compression). By means of the spring which is thus compressed and kept in such a compressed state, a least a portion of energy which is required for moving the ejecting member intensively forward upon ejecting the lancet is stored.

To the contrary, since the distance between a rear flange member 388 and the partition member 384 is increased, the compressed state of the return spring 375 is relaxed (but it is still in the compressed state). The return spring 375 is compressed when the ejecting member 360 is moved forward for the purpose of the ejection and stores an energy so that it can apply a force which moves backward the ejecting member. As a result, in order that a next cassette can move the ejecting member backward, the ejecting member is moved backward after the ejection.

As shown in FIG. 1, the trigger member 370 moves in and along a channel portion 390 inside the housing which portion is formed by opposing walls 392 respectively. In order to facilitate such moving, the trigger member 370 comprises guide portions 394 which can move adjacently to the insides of the wall 392 respectively. The partition member 384 of the ejecting member 360 is fixed by fitting it in to slits 396 provided in each of the guide portions 394, and the ejecting member 360 is arranged such that it can move back and forth inside the trigger member 370. It is noted that it is preferable to provide an ejecting member guide 398 in front of the partition member 384 and the ejecting member 360 extends through a slit 399 provided through the guide 398 so that the smooth movement of the ejecting member 360 is improved.

For the ejecting, when the rear end 384 of the trigger member 370 is pressed forward, the trigger member 370 is moved forward provided that the movement is not to be prevented by means of the stopper 372 as described later. When the trigger member 370 is moved forward, the partition member 384 which is fixed to the trigger member 370 is also moved forward, so that the ejection spring 373 is further compressed (the secondary compression). By such compression, the spring 373 stores an energy which is required for intensely moving the ejecting member forward upon ejecting the lancet.

With the lancet ejecting device according to the present invention, the ejection of the lancet is possible if at least one of the following is available: the compression of the ejection spring 373 when the cassette is fitted into the ejecting device so as to move the ejecting member 360 backward (corresponding to the above mentioned primary compression); and the compression of the ejection spring 373 when the trigger member is moved forward (corresponding to the above mentioned secondary compression). As described above, in addition to the primary compression, the spring member is preferably arranged such that it is further compressed (the secondary compression) when the trigger member is moved forward by pressing the rear end 384. In this case, the secondary compression of the ejection spring 373 in addition to the compression upon fixing the cassette into the ejecting device (the primary compression) makes the force which is acting on the ejecting member 360 increased so that the lancet is intensely ejected. Alternatively, when the ejection spring is strong, a distance over which the ejection spring is primarily or secondary compressed can be shortened, so that a distance over which the rear end 142 of the lancet moves the ejecting member 360 backward and/or a distance over which the trigger member 370 moves so as to release the engagement of the lancet hook portions 144 can be shortened.

The leading end 371 of the trigger member 370 as described above has a function to discontinue the engagement state of the lancet hook portions 144 with the outside of the rear end 210 of the container 102. The trigger member 370 also has being locked portion(s) 506 behind the leading end(s) 371 thereof which portion(s) abuts against rear end(s) 502 of oblique portion(s) 500 of the stopper 372, and preferably against projection(s) provided on the oblique portion(s). When the trigger member 370 is moved forward, the leading end(s) 371 thereof abuts against the lancet hook portions, and when the trigger member is further moved forward, the leading end(s) thereof deforms the lancet hook portions such that those portions approach toward the lancet body.

For the purpose of such deformation, the trigger member is made of a material which is relatively hard and not readily deformed (for example, a hard plastic material such as a polycarbonate). The form of the leading ends 371 is preferably a tapered form or other similar form which diverges forward such that when the leading ends are forced to go over the hook portions after contacting them, the hook portions deform toward the center line (or axis) of the lancet body as a result from that the hook portions are made of a more deformable material than the material of the trigger member.

In consequence, the engaging state of the hook portions 144 is ended, and the lancet 100 is intensely pushed forward and ejected by the ejecting member since the forward force is applied to the rear end 142 of the lancet by the ejecting member.

As seen from FIG. 8, the lower half 204 of the container 102 has convex portions 232 on its both sides which function as catchers. The convex portion is configured such that an engaging portion 510 can be fitted into the convex portion, which engaging portion is provided as a projection on an engaging portion holding member 706 placed in the housing of the ejecting device. The engaging portion 510 is provided on an elastic member 512 (such as a spring, a foam or the like) which is place on the bottom of the housing of the ejecting device. The engaging portion 510 is biased such that when no force is applied to the elastic member 512, that is, when the elastic member 512 is in its expanded state, the engaging portion extends upward. As a result, when the concave portion 232 of the container (or cassette) is present above the elastic member 510, an upward force is acting on the engaging portion 510 while the engaging portion is fitted into the concave portion 232, so that the engaging state is kept between the engaging portion 510 and the concave portion 232, which maintains the engagement relationship between the container 102 and the engaging portion 510. When a downward force is applied to the engaging portion 510, the engaging portion is out of the concave portion, so that the engagement relationship between the container and the ejecting device ends. It is noted that the engagement of the container with the ejecting device is preferably achieved through a connector 374, which is placed at the opening 542 of the front end of the ejecting device.

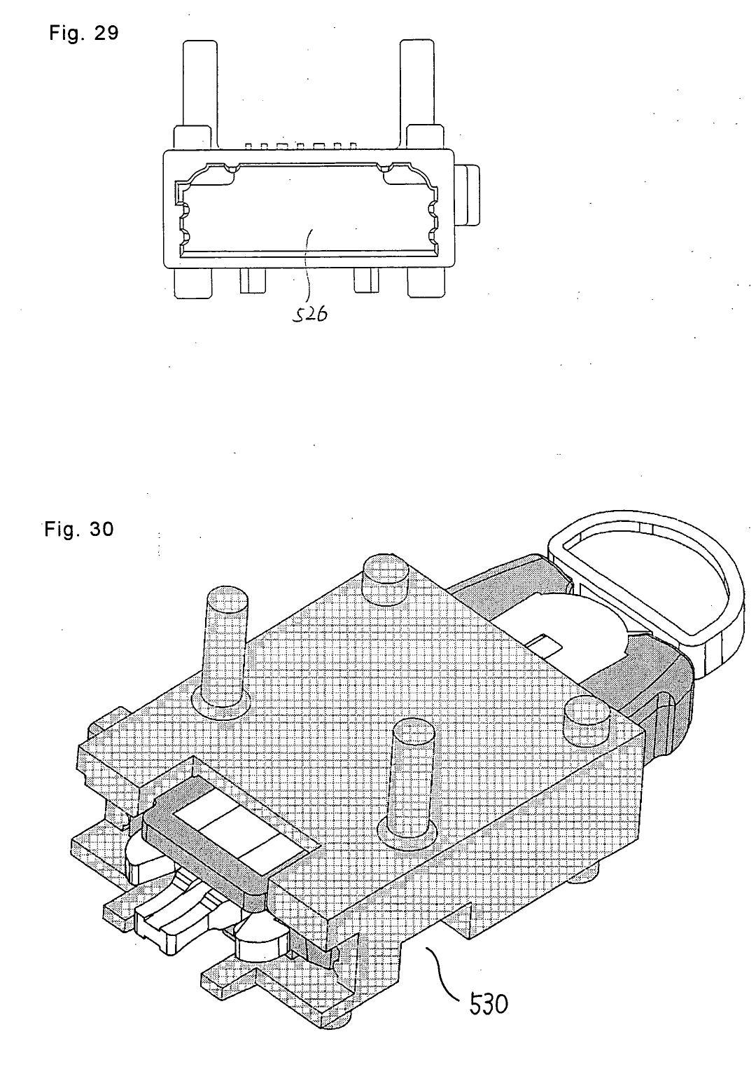

As shown in FIG. 25, the connector 374 has an opening 522 and an opening 526 (see FIG. 29) at its front end 520 and its rear end 524 respectively, and the rear end of the cassette can be inserted from the opening at the front end in to a space inside the connector. At the rear end, there are provided stopper portions 528, which abut against the rear end of the cassette. Thus, when the cassette is inserted in the connector 374 backward from the opening 522 at the front end 522 of the connector 374, the rear end 210 of the cassette abuts against the stopper portions 528, so that the cassette is in the predetermined inserted state while no further insertion is possible. In such predetermined state, the rear end 142 and the hook portions 144 of the lancet protrude outward from the rearmost portion of the rear end of the container 102. Such protrusion allows the hook portions to be readily released from their engagement state by means of the trigger member.

The connector 374 has openings 530 at its lower edges of its both sides. The openings 530 communicates the space inside the connector with the outside of the connector. When the cassette is completely inserted, the concave portions 232 provided at the lower edges of the both sides of the cassette are positioned above the openings 530, so that the engaging portions which engage the cassette with the lancet ejecting device can be fitted into the concave portions 232 of the cassette through the opening 530.

The connector 374 further comprises a locating member(s) 532, which has a function to facilitate arranging an electronic device plate 602 as predetermined. The locating member may be in any appropriate form, and it may be in the form of a column as shown. The inside surface of the connector may have a protruding portion 534 which extends along the insertion direction of the cassette so as to facilitate smooth insertion of the cassette. In the shown embodiment, the side wall has two protruding portions on its inside. Provision of such protruding portion makes not an entire but a portion of the side surface of the container be in contact with the inside wall of the connector, which helps the smooth insertion. The protruding portion may be a series of projections in the form of dots along the insertion direction. Such protruding portion may be provided on only one side surface, and further it may be provided on an inside surface of the upper wall and/or the bottom wall of the container.

Further, a cross section of the cassette which is perpendicular to the insertion direction of the cassette is preferably asymmetric with respect to the up and down direction of the cassette. For example, the cross section has such a shape 536 that only the upper corners of the cross section of the connector are chamfered or curved as shown. Correspondingly, the upper half of the cassette is formed such that the cassette has its upper edge portions which correspond to the chamfered or curved shape of the connector. By doing so, the upper side and the lower side of the connector should correspond to the upper side and the lower side of the cassette in order to insert the cassette into the connector.

As to the cassette according to the present invention, the moving direction of the lancet in the container, that is, the extending direction of the lancet is oblique relative to the horizontal plane, so that the orientation of the cassette as to its up and down direction upon its insertion is important so as to move the ejecting member backward by means of the rear end of the lancet. When the cross section of the connector is asymmetric as described above, the up and down direction of the cassette which is able to be inserted is unambiguously defined, so that the insertion of the cassette may be tried without confirming its orientation. If the insertion is unsuccessful, then the cassette is turned over and the insertion is again tried, and then the insertion becomes automatically successful so that the ejecting member is moved backward as predetermined.

For the purpose of using the cassette, the preparation of using is carried out by fitting an unused cassette into the ejecting device so that the cassette is engaged with the ejecting device. In a preferable embodiment, the lancet ejecting device 400 comprises the connector 374 at the opening 542 at the front end 540 of the device as described above, and the insertion is carried out by fitting the cassette is inserted into the connector. In the embodiment shown in FIG. 2, the connector is designed to be installed on a housing upper half 702 and a housing lower half 704 by means of press fitting.

Upon inserting the cassette as described above, when the cassette is moved backward relative to the ejecting device, the lancet rear end 142 which protrudes from the rear end of the container abuts against and move back the leading end 362 of the ejecting member, and particularly its protruding portion 364. After the rear end of the lancet abuts against the leading end of the ejecting member, when the cassette is further moved backward so as to fit the cassette, a forward force which tries to move the ejecting member forward (i.e. a force by means of the ejection spring 373) is applied to the cassette. When the force which tries to move the container backward (for example, a force applied by fingers by a user) is larger than such forward force, the ejecting member is moved backward by the rear end of the lancet. By moving the cassette backward and making it abut against the ejecting member regardless of the force thus applied to the ejecting member, the spring means is restrained while compressed. The restrained and compressed state of the spring means represents that the spring means can move the ejecting member forward instantaneously when such spring means put in an unrestrained state (i.e. in a condition under which the spring means is able to return to its original form).

When the cassette is moved backward as described above, the engaging portion 510 which is provided in the ejecting device so as to bias to protrude upward is once pressed down, and when the cassette is further moved backward substantially over a predetermined distance from the opening 540 of the front end of the ejecting device, the protruding portion which has been once pressed down is automatically fitted into the concave portion 232 which is formed in the lower half of the cassette, whereby the cassette 104 is engaged with the ejecting device 400 with the ejection spring 373 in the compressed condition. In being fitted as described above, when the connector 374 is provided, the engaging portion 510 fits into the concave portion 232 while passing through the opening 530 of the connector, which finalizes the charge the ejecting device with the cassette.

With the lancet assembly according to the present invention, in the above described state wherein the charge with the cassette is finalized, it is preferable that the front end of the lancet cassette which is engaged with the lancet ejecting device protrudes from the front end of the lancet ejecting device, and a distance between those two front ends is at least 7 mm, preferably at least 10 mm and for example 12 mm. Such protrusion prevents an amount of blood which bleeds upon pricking from depositing to the front end of the lancet ejecting device, which prevents an infection problem by means of blood which is possible when the same lancet ejecting device is used for other patient.

When the lancet ejecting device is not loaded with the cassette, no force is applied to the engaging portion 510 of the ejecting device, so that engaging portion is in the biased state wherein it protrudes into the space of the connector 374 through the opening 530. In the process wherein the cassette is inserted into the space of the connector so as to load it with the cassette, when the rear end of the cassette encounters with the engaging portion which protrudes into the space, the rear end 210 of the cassette once presses down the engaging portion 510 regardless of the force which applies to the engaging portion 510 by means of the elastic member 512 and the rear end goes over the engaging portion. Then, when the concave portion 232 provided on the outer lower edge of the of the outer half encounters with the engaging portion 510 which has been pressed down, the engaging portion 510 automatically goes up because of no application of the pressing down force to the engaging portion 510 and fits into the concave portion 232 of the container through the opening 530 of the connector, so that the engagement relationship between the cassette and the lancet ejecting device is achieved. The manner thus explained above to achieve the engagement relationship is preferable. Inversely, when a downward force which pushes down the engaging portion is applied to the engaging portion by pressing a discharge button 710, the engaging portion 510 is able to be out of the concave portion so that the engagement relationship between the cassette and the lancet ejecting device is disconnected.

In the state wherein the container is engaged with the lancet ejecting device as described above, the engagement relationship between the cassette and the lancet ejecting device cannot be ended unless the engaging portion moves downward. Also, in such engagement state, the rear end of the lancet protruding from the opening at the rear end of the container is pushing the leading end of the ejecting member backward. In other words, this state is a state wherein the forward force of the spring is applied to the lancet through the rear end of the lancet.

It is noted that even though the forward force is applied to the rear end 142 of the lancet, the lancet does not move and in its static condition since the lancet hook portions 144 are engaging with the outside of the rear end 210 of the lancet. Therefore, the forward movement of the ejecting member 360 is prevented by the lancet hook portions 144. However, when the engaging condition of the lancet hook portions is discontinued, the lancet 100 and thus the ejecting member 360 becomes in a condition to be able to move forward, so that the force applied by the ejection spring 373 moves the ejecting member instantaneously forward and also the lancet which is abutting against the ejecting member moves instantaneously forward, whereby the ejection of the lancet is carried out.

The lancet assembly according to the present invention preferably comprises an electronic device 600 which receives electronic signals from the sensor element 118, and processes the signals as predetermined so as to provide and display a converted figure (a blood glucose level) which relates to an intended measurement. An example of such electronic device is one used in a blood glucose level measuring device which processes electric signals from the sensor element to convert them into a blood glucose level. Such electronic device preferably comprises a display which indicates a measurement such as a blood glucose level.

In one embodiment, such electronic device is arranged on the electronic device plate 602, which is placed in the housing. As shown in FIG. 1, the plate is fixed to the housing by placing it over the cover member 377 which covers the trigger member 370. The electric signals are transferred from the sensor element mounted on the lancet cassette according to the present invention through terminals 119 of the sensor element to terminals (not shown) of the electronic device, and such signals are converted to a measurement after their processing, and the measurement is indicted on the display provided on the ejecting device.

Electronic members and electronic circuits (including the display) which calculate and indicate the measurement from the electric signals as described above are known, and those known for the blood glucose level measurement can be employed. For example, those used in Diameter α (commercially available from Arkray) as a blood glucose level measurement apparatus can be incorporated as the electronic device into the lancet ejecting device according to the present invention.

As to the ejecting device according to the present invention, the ejecting member is designed such that a moving direction of the leading end of the ejecting member forms an angle with the moving direction of the lancet during the ejection of the lancet. As a result, in the process of the ejection, an area where the rear end of the lancet 142 abuts against the leading end 362 of the ejecting member gradually decreases, so that the engagement relationship between those is finally discontinued as described above. In other words, the ejecting member is designed such that the moving direction of its leading end diverges from the moving direction of the lancet.

For example, an abutment region where the rear end 142 of the lancet abuts against the leading end 362 of the ejecting member extends along a thickness direction of the lancet such that the region gradually shifts downward relative to the rear end of the lancet when the rear end of the lancet and the leading end of the ejecting member moves forward while keeping the abutment relationship between them, and finally the abutment relationship cannot be maintained any more. At the time when the maintenance of the abutment relationship becomes impossible, the separation of the rear end of the lancet from the ejecting member occurs, so that the lancet is ejected forward, and the leading end of the ejecting member moves forward below the pricking direction. Such abutment region may be with the form of the protruding portion 364 provided on the leading end 362 of the ejecting member.

The embodiment where one moving direction diverges from the other moving direction is based on a relative relationship between the two moving directions. As described above, the moving direction of the lancet, that is, the pricking direction is obliquely upward with respect to the horizontal plane which corresponds to the extending plane of the sensor element. The moving direction of the leading end of the ejecting member is obliquely downward relative to the moving direction of the lancet. Such moving direction of the leading end of the ejecting member may be obliquely upward relative to the horizontal plane, horizontal, or obliquely downward relative to the horizontal plane.

In a particularly preferable embodiment, as shown in FIG. 59, the lancet moves obliquely upward relative to the horizontal plane (i.e. the direction indicated with the alternate long and short dash line A in FIG. 59), and the ejecting member moves horizontally forward or obliquely, downward and forward (i.e. the direction indicated with the alternate long and short dash line C in FIG. 59). In this embodiment, the moving directions of the lancet and the leading end of the ejecting member are not parallel but substantially intersects with each other. An extent of such intersection is expressed by an angle β which is formed by the moving directions (which appear when viewing the moving of the lancet from its just side). The angle β is a smaller one of angles formed by the intersection. Since the angle α is an angle formed by the pricking direction (which is indicted with the alternate long and short dash line A in FIG. 59) and the horizontal plane (which is indicted with the alternate long and short dash line B in FIG. 59), the angle β is at least the same as the angle α. When β=α, the moving direction of the leading end of the ejecting member is horizontal and forward.

The angle β is preferably from 6° to 20°, more preferably from 10° to 17°, particularly from 12° to 15°, and for example 13°. When the moving directions of the lancet and the leading end of the ejecting member form the angle as described, the discontinuation of the engagement relationship between the rear end of the lancet and the ejecting member becomes easy. Also, the ejecting member is surely moved backward when a cassette containing an unused lancet is fitted into the ejecting device. Further, the ejecting member is surely not moved backward when a cassette containing a used lancet is fitted into the ejecting device.

In addition, the obliquely upward movement of the lancet discontinues the engagement relationship between the rear end of the lancet and the leading end of the ejecting member. It is important to ensure that until such discontinuation, the rear end of the lancet and the leading end of the ejecting member move along the predetermined directions while they are abutting against each other. The moving direction of the lancet is ensured by the inner space extending obliquely upward with a predetermined angle which space is defined by the inner surface 201 of the container upper half and the inner surface 215 of the container lower half as described above. The moving direction of the leading end of the ejecting member is ensured by a guide portion 550 which directs the leading end of the ejecting member along a predetermined direction. In one embodiment, the leading end 362 of the ejecting member 360 has side protruding portions 367 preferably in the vicinity of or in front of a base portion of the protruding portion 364, and the guide portions 550 which are provided on a lower outside surface of the connector 374 moves the side protruding portions 367 along a predetermined direction. The guide portion has an inclined surface which shifts a position of the leading end of the ejecting member downward as the member moves forward.

It is preferable that the side protruding portion 367 is located below the guide portion 550 even though the lancet rear end 142 is kept abutting against the protruding portion 364 of the leading end of the ejecting member. As a result, when the ejecting member moves forward, its leading end 362 moves smoothly, gradually, obliquely, downward and forward on the inclined surface of the guide portion 550.

As described above, the ejecting member is advanced up to its possible front most position and then moved back by the return spring 375 while the ejecting member is moved forward and the lancet is ejected. However, even when the ejecting member has been moved back, the leading end 362 of the ejecting member is preferably located below the above described guide portion 550. In order to facilitate the movement of the leading member of the ejecting member 360 as described above, it is preferable to form the ejecting member by providing an elasticity to a portion the ejecting member such that substantially only the leading end of the ejecting member can move obliquely downward while the ejecting member as a whole moves horizontally. In order to form such ejecting member, the ejecting member 360 is made of a plastic material (such as a polyacetal resin) and a portion 369 thereof is made thinner so that such portion has the elasticity while the remaining portion is relatively rigid.

It is noted that since the lancet rear end does not abut against and move backward the leading end of the ejecting member even though a used cassette is fitted into the cassette receiving member through its opening by error since the lancet rear end 142 is contained inside the container.

The lancet ejecting device according to the present invention comprises a stopper 372 which locks the trigger member. The term “lock” used herein means that the trigger member cannot be moved substantially forward so that the lancet cannot be ejected except when pricking is carried out. With the stopper, only when a cassette which contains a usable lancet (that is, a cassette in the state wherein the lancet hook portions 144 and the lancet rear end 142 protrude from the opening 212 at the rear end of the container) is fitted into the ejecting device, the locking state is released, so that it becomes possible to press so as to move the trigger member 370 forward. Concretely, the trigger member 370 can move forward only when the ejecting member 360 is kept in its retracted state by means of the lancet rear end 142.

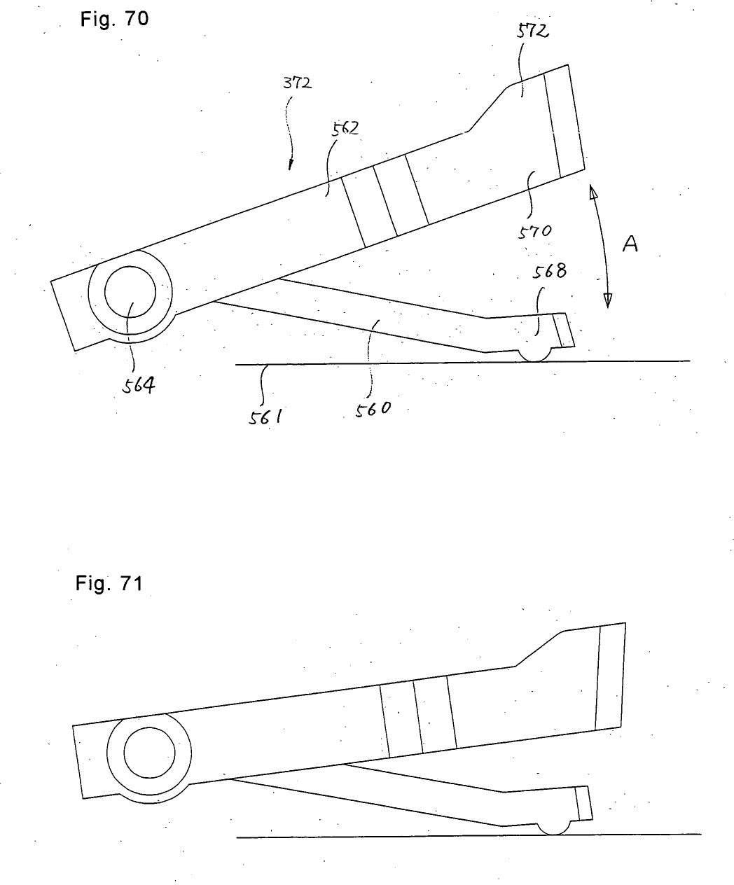

The stopper 372 comprises a base portion 560 which is located on the bottom of the housing 561 of the ejecting device and an oblique portion 562 which is inclined relative to the base portion. As shown in FIG. 70, these two portions are integrally connected at their front end 564 while their rear ends 568 and 570 are separated from each other. When a force is applied to the oblique portion so as to push the portion downward, the stopper is elastically deformed such that the separated rear end portions 570 and 568 of the oblique portion and the base portion approach to each other as shown in FIG. 71, and when such force is released, the stopper elastically returns to substantially its original form as shown in FIG. 70. Such stopper is located below a pass along which the leading end of the ejecting member moves, and functions depending on the relationship between positions of the leading end and the stopper.

When the stopper is in its original form as shown in FIG. 70 because the leading end 362 of the ejecting member is located above or near the front end 564 of the stopper so that no force is applied which presses the oblique portion downward, a protruding portion 572 provided on the rear end of the oblique portion abuts against a being locked portion 506 of the trigger member so that the protruding portion 572 prevents the trigger member from moving forward.

On the other hand, when the ejecting member 360 is moved backward by means of the lancet rear end 142, the leading end 362 of the ejecting member moving backward tries to move horizontally and backward. In the case wherein such leading end 362 approaches to the rear end 570 of the stopper oblique portion 562 or the vicinity thereof, the leading end of the ejecting member contacts with the oblique portion 562 and moves on the oblique portion while applying a downward pressing force (a component force) to the portion. That is, the leading end 362 of the lancet ejecting member applies a force to the oblique portion 562 of the stopper so as to elastically deform the stopper so that the rear end 570 of the oblique portion and the rear end 568 of the base portion which have been spaced from each other approach to each other as shown in FIG. 71. In the state wherein the ejecting member is restrained while being moved back (that is, the state wherein the cassette is engaged with the ejecting device), the ejecting member 362 is held while it applies the force to the oblique portion 562 of the stopper as described above.

In such state, when a force is applied so as to move the trigger member 370 forward, the being locked portion 506 of the trigger member which tries to move forward passes above the oblique portion 562 without colliding the protruding portion 572 provided on the rear end of the oblique portion since that portion 562 is pushed downward. As a result, thus having passed trigger member 370 is able to release the engagement state of the lancet hook portions.

As clearly seen from the above description, the stopper has to deform elastically, and it may be of any form and made of any material as far as the stopper provides the above described function. In a preferable embodiment, the stopper is produced by molding a resin, and it has a V-shape when viewing from its side as shown in FIG. 70. In this case, the corner portion of the “V” shape corresponds to the front end of the stopper and the hypotenuses of the “V” shape correspond the base portion and the oblique portion respectively. The base portion and the oblique portion may be of any form (such as a plate, a strip, a rod or the like) as far as they provide the function as the stopper.

### Embodiments to Carry Out the Invention

The lancet assembly according to the present invention is schematically shown in FIG. 1 in an exploded view. The cassette 104 of the present invention is comprised of a container 102 which houses the lancet 100, while the drawing surrounded by the alternate long and short dash line shows the state before assembling, and the state after assembling is indicated with the arrow. In the shown embodiment, the sensor element 118 is placed on the outer surface 207 of the cassette 104. It is noted that the sensor element has an opening 121 behind its front end 120 as shown. The opening 121 can facilitate the capillary phenomenon by discharging air when absorbing an amount of blood from the front end 120.

The lancet ejecting device according to the present invention comprises the lancet ejecting member 360, the trigger member 370, the stopper 372, the connector 374 and the engaging portion holding member 706 as well as other parts associated with those members in the housing 700 which is formed with the housing upper half 702 and the housing lower half 704. In the housing 700, the cover member 377 which covers most of the trigger member 370 is installed on the lower half 740 by means of press fitting, and the electronic device plate 602 is placed on the cover member. The electronic device 600 is located on the plate 602.

The engaging portion holding member 706 is placed on the elastic member 512 (such as a spring, an elastic form or the like) which is disposed on the lower half 704. The stopper 372 is located behind the engaging portion holding member 706, and the ejecting member 360 is placed such that its leading end 362 is located above the oblique portions 372 of the stopper 372. The ejection spring 373 and the return spring 375 are placed around a rear half portion of the ejecting member 360, and there is provided the partition member 384 between the springs which is movable along the longitudinal direction of the ejecting member. The compression states of these springs depend on the relative position of the partition means 384 which is placed between the flange member 386 located around the center of the ejecting member 360 and the flange member 388 located rear end of the ejecting member 360.

Most part of the trigger member 370 is located between the walls 392 of the channel portion of the lower half 704 and the outside of the ejecting member 360 so that the trigger member is able to move back and forth. The rear end 384 of the trigger member protrudes outward from the opening of the rear end of the housing. In order that the trigger member returns to its original state after the rear end is pressed forward, the return spring 376 is placed between the rear end 379 of the cover member fixed to the housing and the trigger member (particularly the protrusion 381 provided on the flange portion 381).

The partition member 384 is fitted into the slits 396 of the trigger member, and the forward movement of the trigger member 370 moves the partition member 384 forward so that the spring 373 is subjected to the secondary compression.

The connector 374 is placed over the engaging portion holding member 706 and the stopper 372 by means of the press fitting into the front end 540 of the housing 700 such that its openings 530 provided on its both sides are aligned with the protruding portions 510. It is noted that the posts 622 provided on the upper surface 620 of the connector 374 pass through holes 624 of the plate 602, so that the plate 602 is located within the housing.

The lancet ejecting device according to the present invention comprises the cassette discharge mechanism, which comprises a discharge slide button 710, an elastic member 712 and a pressing member 714. The button 710 is connected with the elastic member 712 through the upper half 702. Concretely, the connection is achieved by fitting leg portions 716 provided on a lower surface of the button 710 into slits 718 provided on the elastic member 712 which is located below the upper half 702 by for example snap fitting. The button 710 thus connected can be moved back and forth in the opening 720 provided through the upper half.

The pressing member 714 comprises the leg portions 724 which pass through the openings 722 formed through the plate 602. When a downward force is applied to the leg portions, the leg portions can press the engaging portion holding member 706 downward which is biased upward by means of the elastic member 512. Such downward force is applied to the pressing member 714 by sliding the button 710 forward so as to move the elastic member 712 forward. For such purpose, the elastic member 712 and the pressing member 714 have inclined surfaces 726 and 728 respectively. These inclined surfaces can contact with each other and when the inclined surface 726 is moved horizontally relative to the inclined surface 728 so as to form an angle between these surfaces (that is, moved not parallel to each other), the inclined surface 726 presses the inclined surface 278 downward, so that the pressing member 714 moves downward so as to press the engaging portion holding member 706 downward regardless of the elastic member 512.

In this way, moving the button 710 forward moves the engaging portions 510 downward, so that the engaging state of the engaging portions 510 with the concave portions 232 of the cassette is discontinued, whereby the cassette becomes free, that is, the cassette can be taken out from the device into which the cassette has been fitted. In the shown embodiment, the elastic member 712 has downward protruding members 719 behind the slits 718. As a result, when the slide button 710 is pushed forward subsequently, the downward protruding portions 719 of the elastic member 712 pushes the cassette forward which has become free, so that the cassette which has been fitted into the ejecting device is discharged out of the openings 522 at the front end 520 of the connector.

It is noted that the elastic member 712 has arm form spring portions 730 on its both sides such that their end portions are engaged with the inside of the upper half 702. In the shown embodiment, their end portions are curled, and the curled portions are engaged with projections provided on the inside of the upper half 702. The end portions remain engaged with the projections, so that the spring portions 730 are elastically deformed when the button 710 is moved forward by a force applied by for example a finger tip. When such force is released, the elastic member 712 returns to its original form due to the elasticity of the spring portions so that the button is automatically moved back.

After locating the plate 602 on the connector 374 and the cover member 377, the pressing member 714 is placed on the plate 602, over which the upper half 702 is placed, so that the ejecting device is completed. It is noted that when the various members or parts are placed as described above, any appropriate manner may be used so as to fix them if it is necessary to fix them. In the shown embodiment, press fitting may be used for the installation.

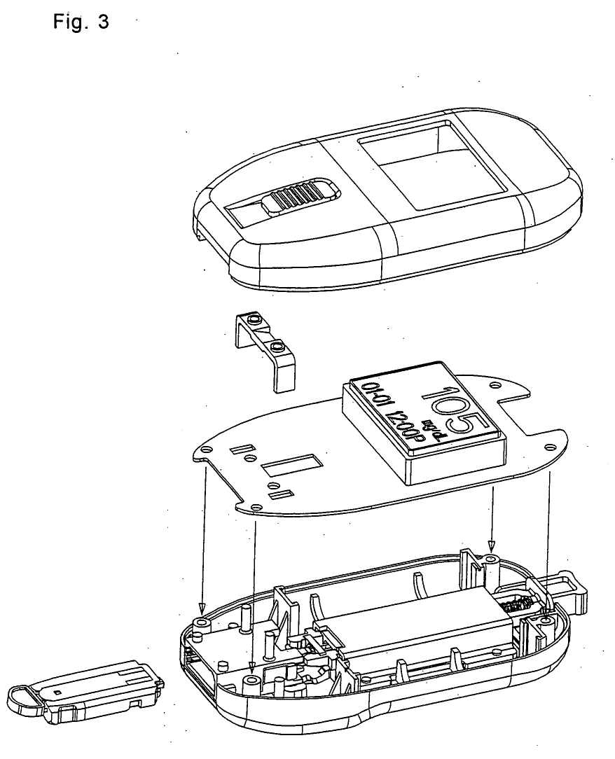

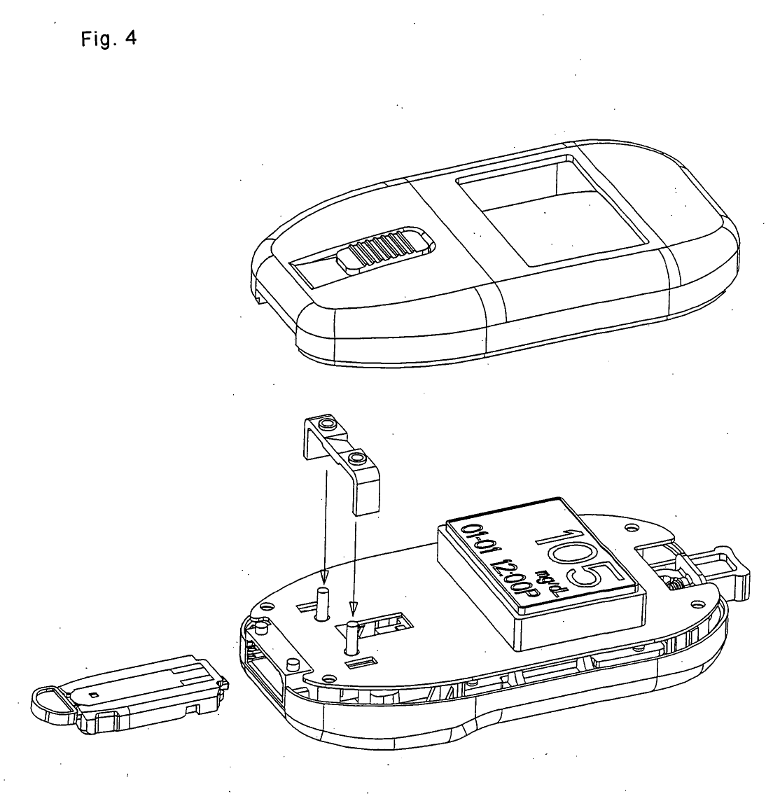

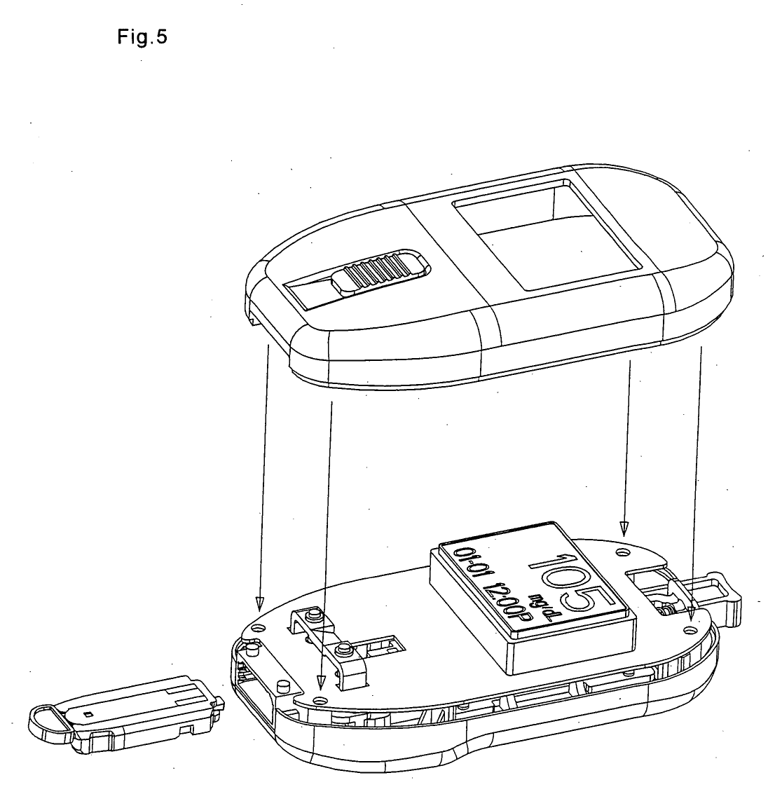

FIGS. 3, 4 and 5 schematically show in perspective views a sequence of placing the plate 602, on which the electronic device has been mounted beforehand, on the lower half 704 on which the stopper 372, the protruding portion holding member 706, the ejecting member 360, the trigger member 370, the connector 374 and the cover member 377 have been placed (see FIGS. 2 and 3); then placing the pressing member 714 while aligning it with the protruding portion holding member 706 (see FIG. 4); and further placing the upper half 702 on which the discharge button 714 has been fixed beforehand on the lower half 704 (see FIG. 5).

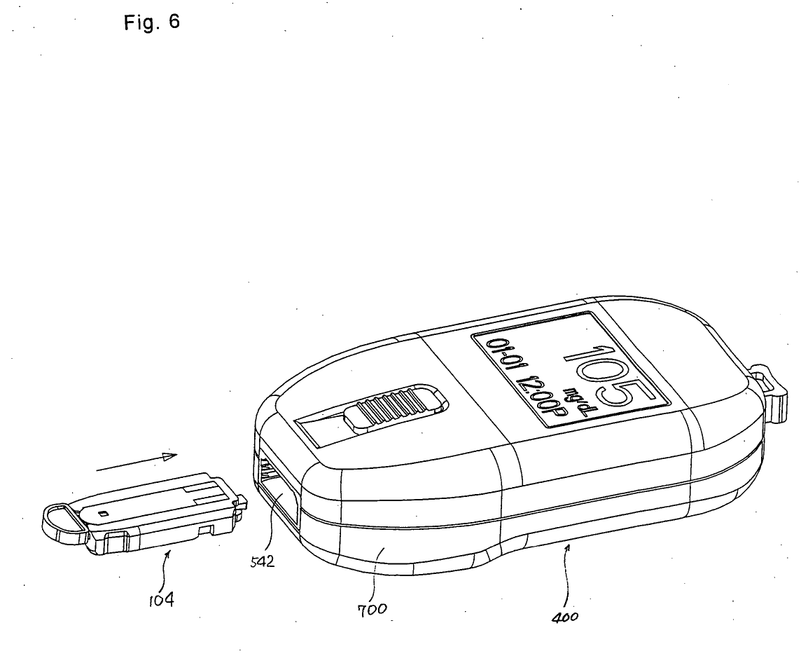

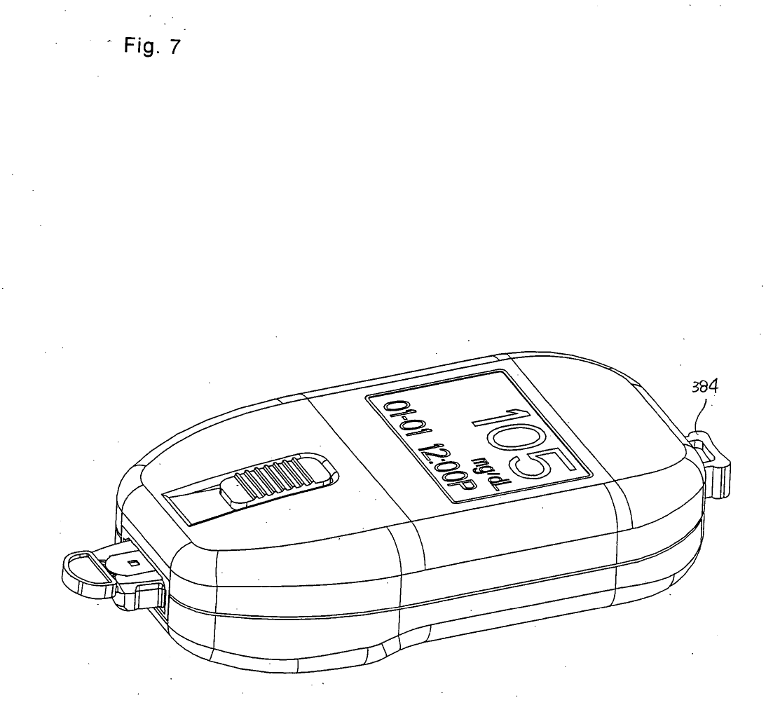

The lancet ejecting device 400 which is assembled as described above is schematically shown in a perspective view in FIG. 6. The ejecting device is loaded with the cassette 104 through the opening 542 at the front end 540 of the ejecting device 400, whereby the cassette is engaged with the device. The cassette thus engaged is schematically shown in FIG. 7 in a perspective view. In the shown embodiment, the ejecting member 360 is restrained in its moved back condition by means of the cassette rear end 142.

Thereafter, by pulling the tab portion 150 in the embodiment shown in FIG. 7, the tip portion of the pricking member is exposed as shown in FIG. 16 in the cassette. Then, the predetermined portion is pressed against the front end 206 of the cassette, and by pressing the rear end 384 of the trigger member, the lancet can be ejected.

FIGS. 8 to 13 schematically show in perspective views a sequence of assembling the lancet cassette according to the present invention. First, the container preform 220 shown in FIG. 8 is prepared. The preform may be prepared by injection molding of for example a polystyrene resin, a polyethylene resin, a polypropylene resin or a copolymer of monomers for the above mentioned polymers. The shown preform is molded such that the upper half 202 which constitutes an upper part of the container and the lower half 204 which constitutes an lower part of the container are connected together by means of the hinge portion 218.

Then, as shown in FIG. 9, the lancet as shown in FIG. 15 which has been prepared beforehand is arranged on the lower half 204. The arrangement of the lancet is carried out such that the lancet hook portions 144 engaged with the outside of the rear end 210 of the container, the pullout preventive portions 146 are adjacent to the inside of the rear end 210, and the tab portion 150 extends outward from the opening 208 at the front end of the container.

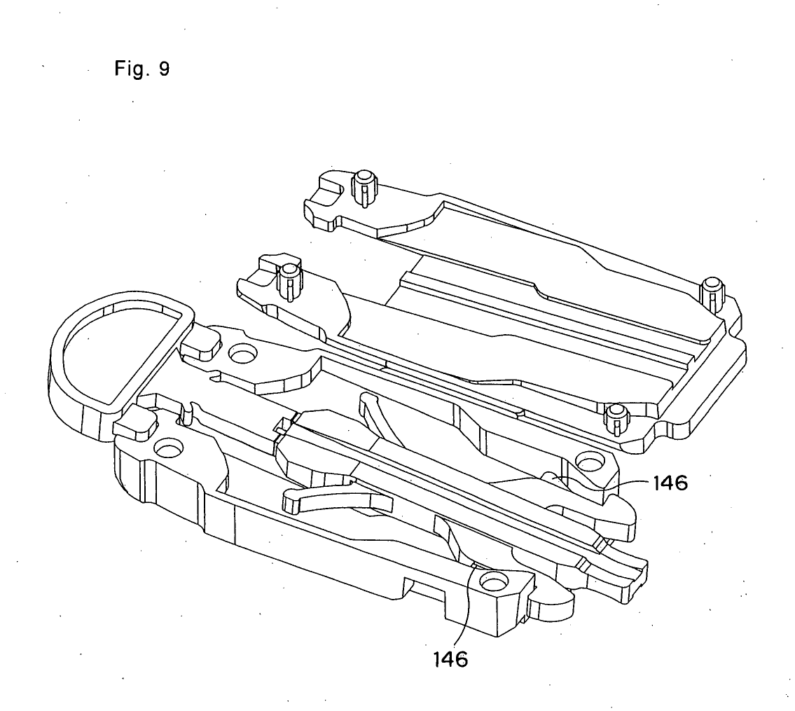

Thereafter, the preform is folded around the hinge portion as shown in FIGS. 10 and 11, and finally the upper half 202 and the lower half 204 are opposed and press fitted to each other so as to integrate them together as shown in FIG. 12, so that the cassette 104 which contains the lancet 100 is obtained. Then, if necessary, the sensor element 118 is placed on the upper surface 207 of the cassette 104 as shown in FIG. 13, so as to provide the cassette having the sensor element (see FIG. 14). The ejecting device according to the present invention is loaded with such cassette as shown in FIG. 6.

The lancet 100 according to the present invention is schematically shown in FIG. 15 in a perspective view and in FIG. 16 in a side view. It is noted that FIG. 16 shows a state wherein the shield member 110 has been removed. Such lancet may be produced by injection molding of a resin such as a polyethylene resin, a polypropylene resin or the like while the pricking member 106 is inserted.

In the present invention, the moving direction of the lancet 100 within the container, and thus the pricking direction is not parallel to the horizontal plane which is defined by the sensor element 118, but the moving direction and the horizontal plane intersect to form the angle α. Such state is schematically shown in FIG. 17 in a cross-sectional view, in which a finger tip 800 is shown as the predetermined portion. A thickness of the front end portion 107 of the lancet body 108 becomes gradually smaller toward the front end thereof, so that the front end portion 107 is not in contact with the sensor element 118 before pricking by the lancet 100. It is readily seen that if the thickness of the front end portion were the same as that of the remaining portion, the front end portion pushes the sensor element upward.

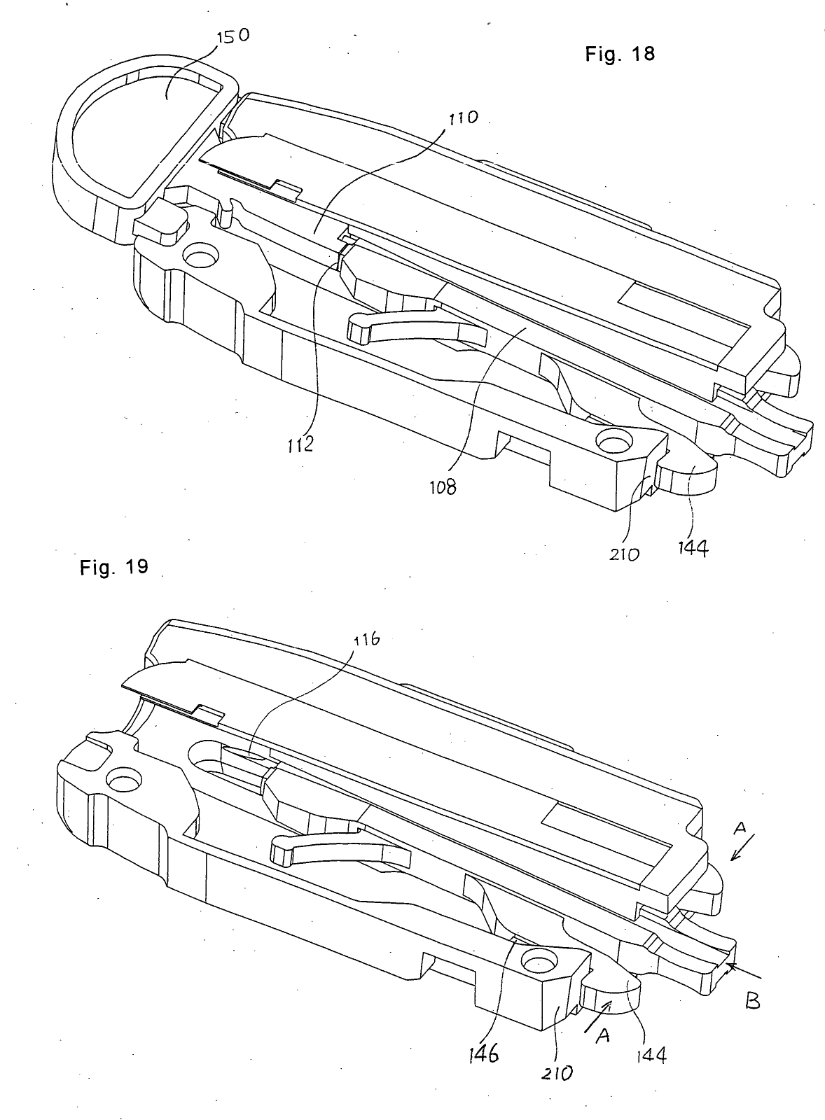

The sequence of the lancet moving in the container is schematically shown in FIGS. 18, 19, 20 and 21 in perspective views upon pricking. It is noted that in the drawings, the near side half of the upper half which forms the upper part of the cassette and the near side half of the sensor element are cut away for the purpose of readily understanding the inside of the cassette.

FIG. 18 shows the state wherein the lancet still has the shield member 110 in the cassette which contains the unused lancet, and FIG. 19 shows the state wherein the shield member 110 has been removed from the state of FIG. 18, so that the tip portion 116 of the pricking member 106 is exposed. In FIGS. 18 and 19, the lancet hook portions 144 engage with the outside of the rear end of the container.

In the state of FIG. 19, when the lancet hook portions 144 are pushed by the trigger member so that they are moved inward as shown with the arrows A, the lancet rear end 142 is intensely pushed forward as shown with the arrow B by the ejecting member so that it is moved forward.

As to thus intensely having moved lancet 100, its retracting portions 148 hit against the collision portions 205 inside the container and then elastically bend. Such state is shown in FIG. 20. Such hitting may be just prior to, after or simultaneous with pricking.

Since thus bending retracting portions 148 try to return to their original forms, they decrease the momentum of the lancet which is going to prick so as to alleviate the impact upon pricking as well as move the lancet backward. Alternatively, when hitting is simultaneous with or after pricking, they move the lancet backward. The anteroposterior relationship in time between hitting and pricking may be any appropriate one. For example, when the momentum of the ejected lancet is large, it is preferable that the momentum is decreased a little prior to pricking. When no alleviation of the momentum is required, hitting is preferably simultaneous with or after pricking.

Elastically returning of the retracting portions 148 to their original forms moves the lancet 100 backward, so that the tip portion 116 of the pricking member and the lancet rear end 142 are contained within the container as shown in FIG. 21, and also the lancet hook portions 144 are substantially contained within the container. It is preferable that the lancet is housed in the container substantially statically.

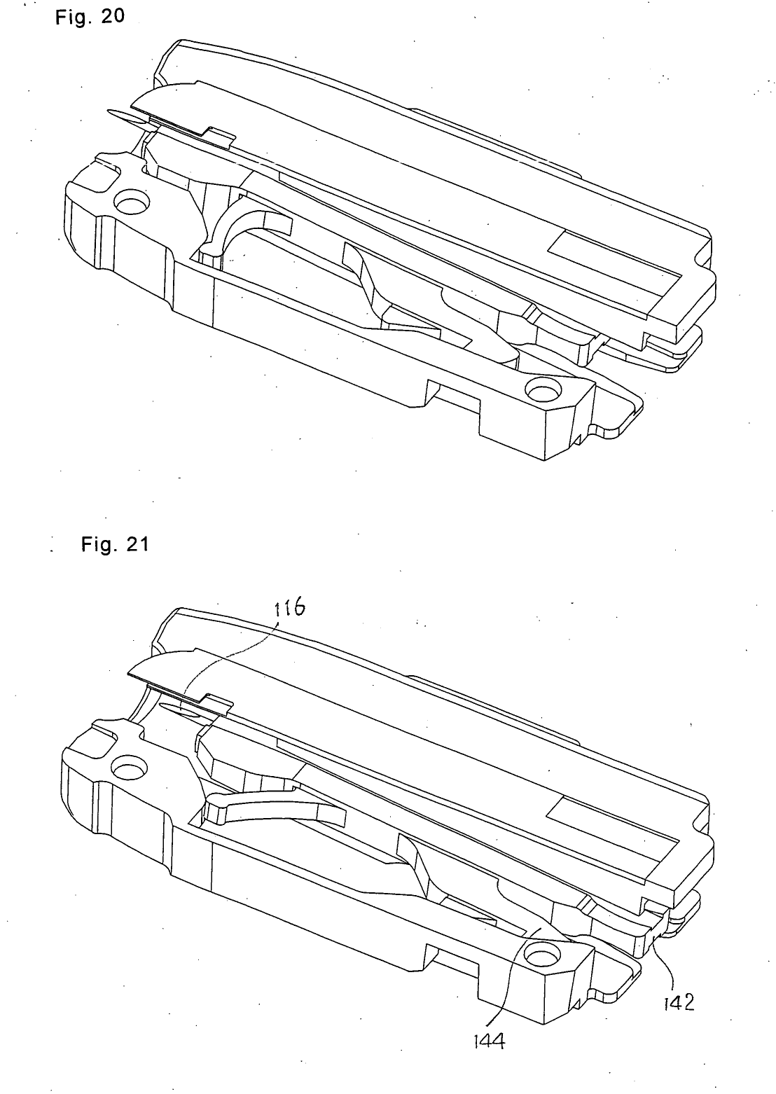

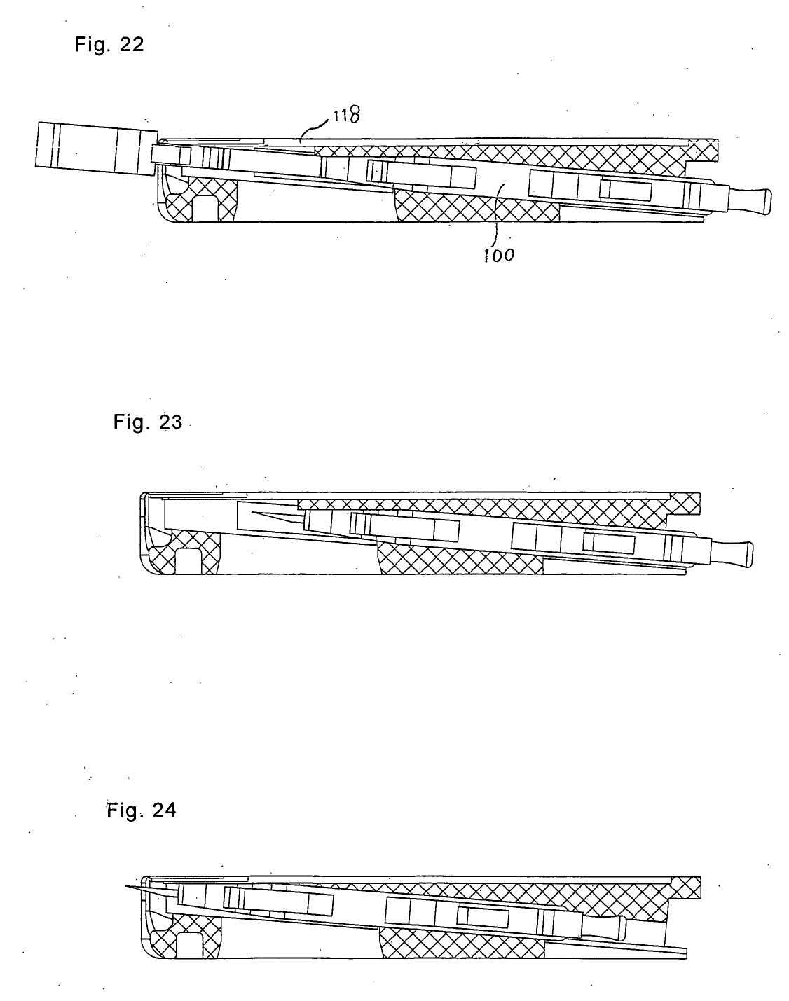

FIGS. 22, 23 and 24 show side cross-sectional views which correspond to FIGS. 18, 19 and 20 respectively. From the drawings, it is readily understood that how the lancet is in the container. Also, it is seen from the drawings that the moving direction of the lancet is inclined as to the extending plane of the sensor element.

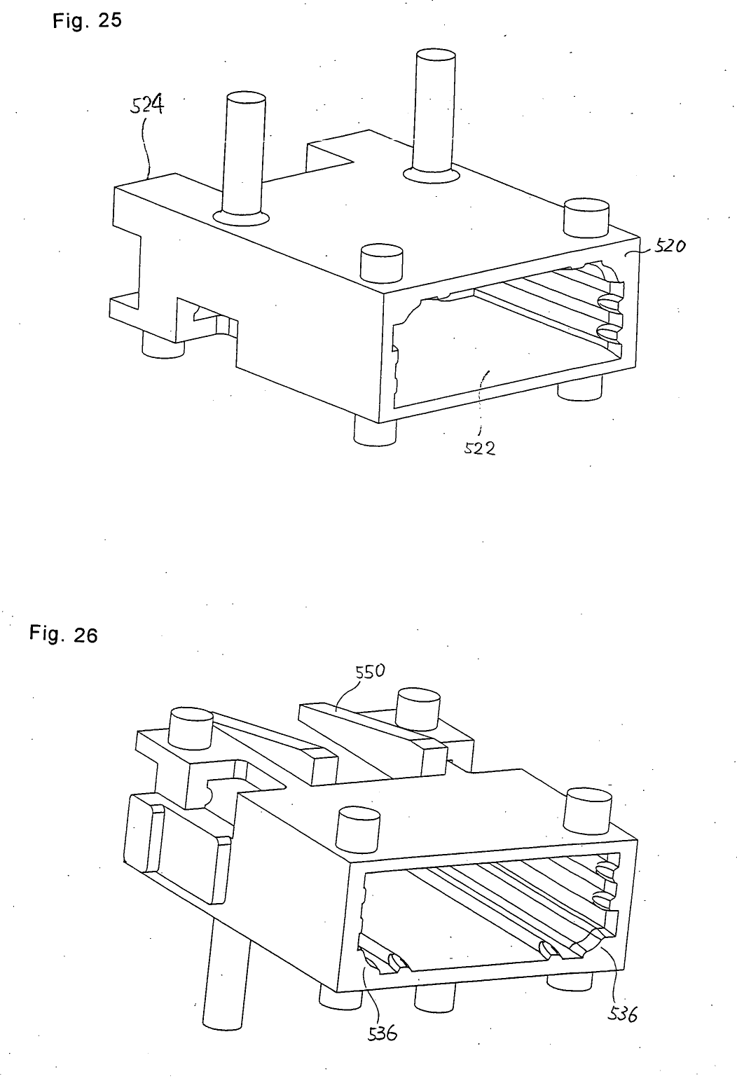

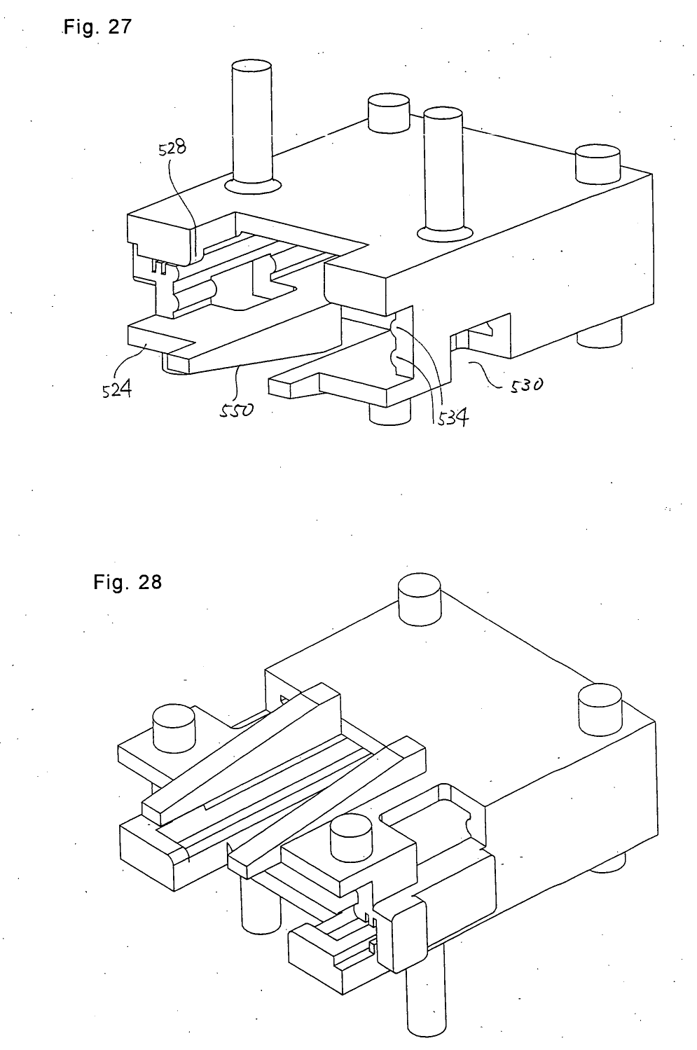

FIG. 25 shows the connector 374 in a perspective view. In the shown embodiment, the near side opening 522 is an opening through which the cassette is inserted. FIG. 26 shows in a perspective view the connector which is achieved by turning over the connector of FIG. 25. FIG. 27 shows e connector when viewing it from its rear side as shown with in the arrow A in FIG. 25. FIG. 28 shows in a perspective view the connector which is achieved by turn ing over the connector of FIG. 27. FIG. 29 shows the connector in an elevational view when viewing it from its rear side.

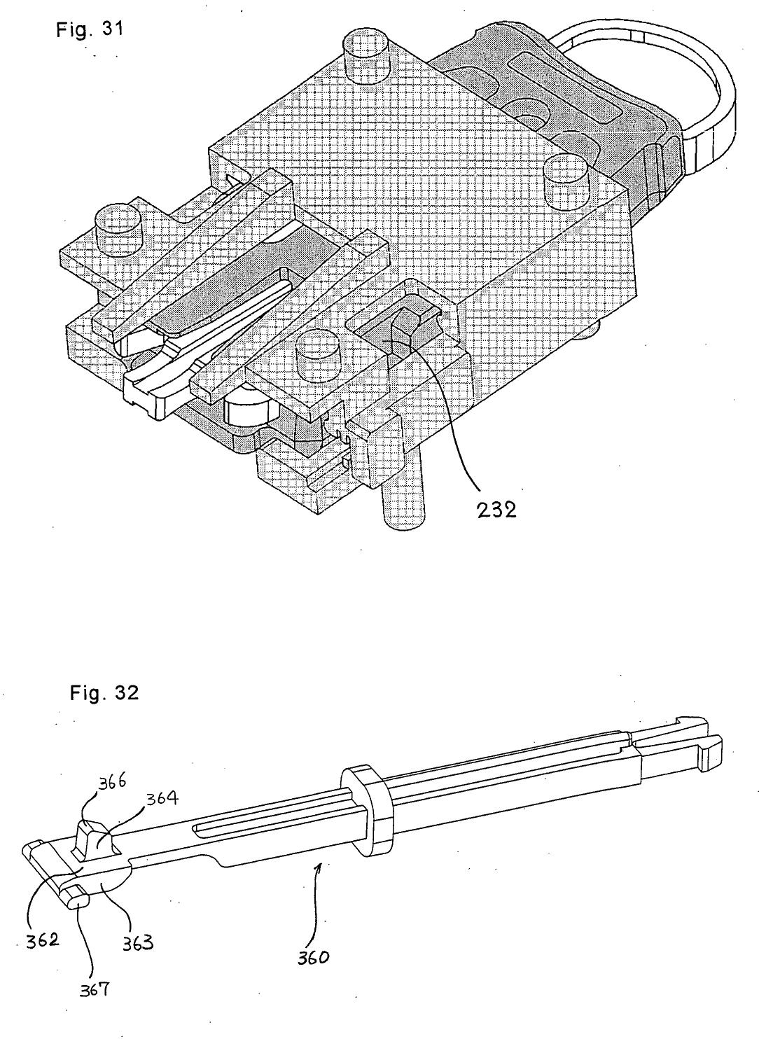

FIG. 30 shows the connector 374 into which the cassette 104 has been fitted. FIG. 30 shows the connector shown in FIG. 27 into which the cassette has been inserted while FIG. 31 shows the state which is achieved by turning over the connector of FIG. 30. As seen from FIG. 31, the concave portions 232 which are provided in the lower sides of the container are aligned with the openings 530 of the connector.

FIG. 70 schematically shows a side view of the stopper 372. As described before, the stopper is elastically deformable as shown with the two-headed arrow A, and when a force is applied to press the oblique portion 562 downward, the stopper deforms such that the rear ends 568 and 570 approach to each other as shown in FIG. 71. As seen from FIGS. 70 and 71, the stopper has a V-shape as a whole when viewing it from its side, and an opening extent of the two sides of V-shape changes. When the state shown in FIG. 70 is changed to the state shown in FIG. 71, the position (or level) of 572 is lowered.

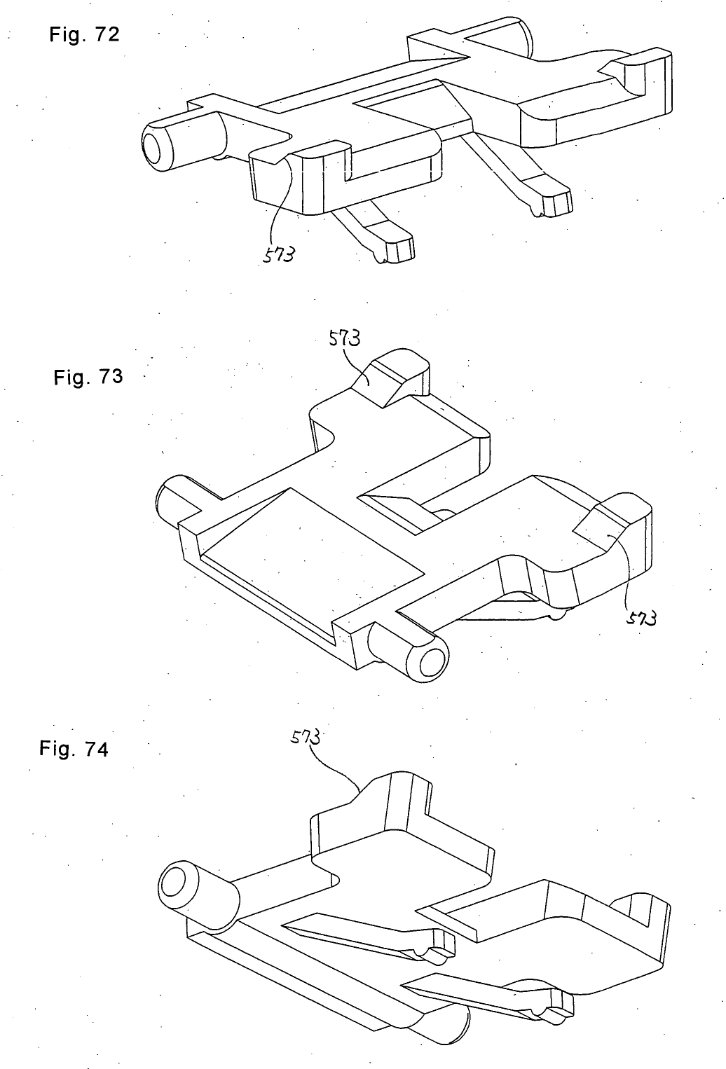

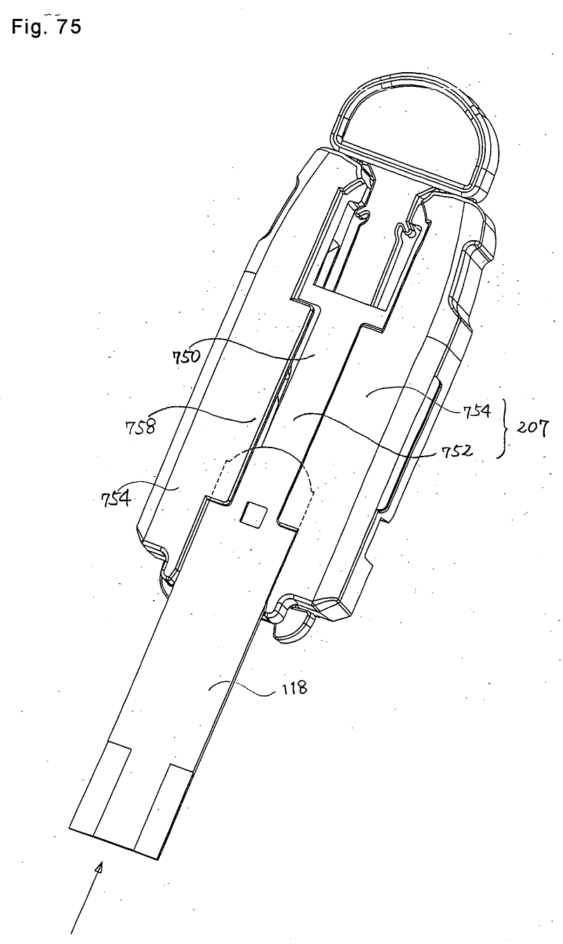

FIGS. 72, 73 and 74 schematically show the stopper 372 in its perspective views. FIG. 72 shows the state when the stopper 372 is viewed from its oblique, upper and rear position. FIG. 73 is substantially the same as that shown in FIG. 1. FIG. 74 shows the state when the stopper 372 is viewed from its oblique, lower and rear position. The oblique portion(s) 562 and the base portion(s) 560 may be any ones as far as they form the V-shape which is elastically deformable. For example, the oblique portions 562 and the base portions are formed of two elongated strips which are spaced or they are in the sheet form (that is, a form corresponding to strips which extend while being integrated). The elastic deformation may be achieved by molding the stopper 372 by using a resin (such as a polyacetal resin, a polyamide resin or the like), wherein the integration of the front end of the oblique portion with the front end of the base portion is simultaneously achieved. Alternatively, the oblique portion and the base portion are pivotally connected, and a spring is placed between the rear ends of the oblique portion and the base portion so that they can function as the stopper.

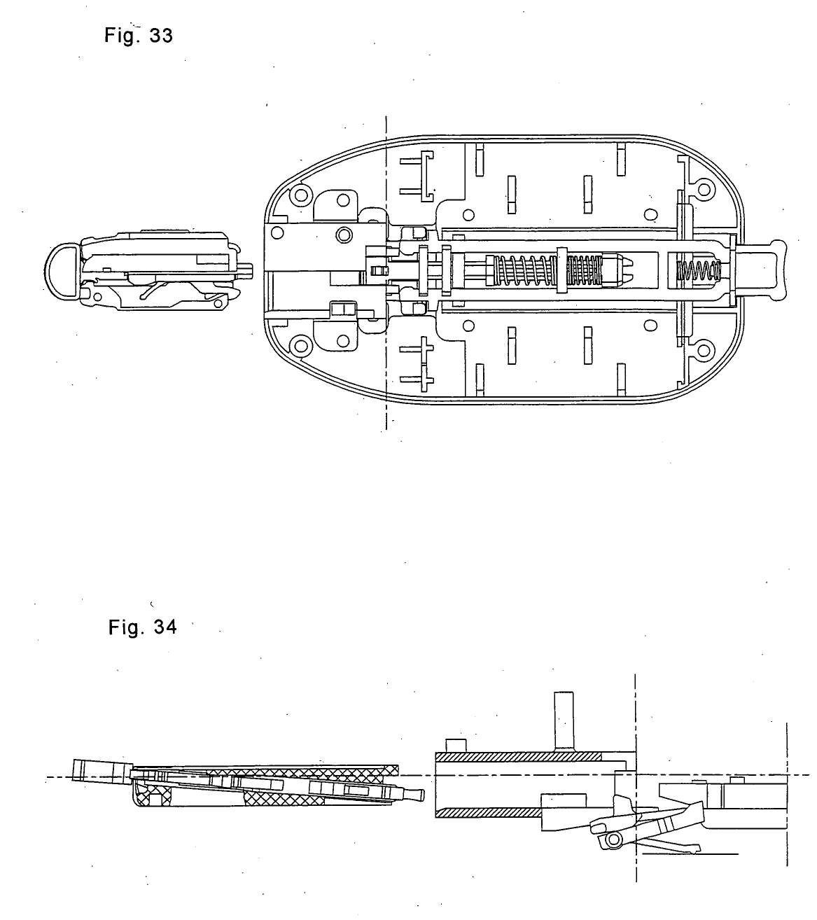

The sequence of loading the ejecting member with the cassette is schematically shown in FIGS. 33 to 49 in plan views and side cross-sectional views of the cassette and the lower half of the housing as well as optionally in perspective views of the ejecting device into which the cassette is fitted.

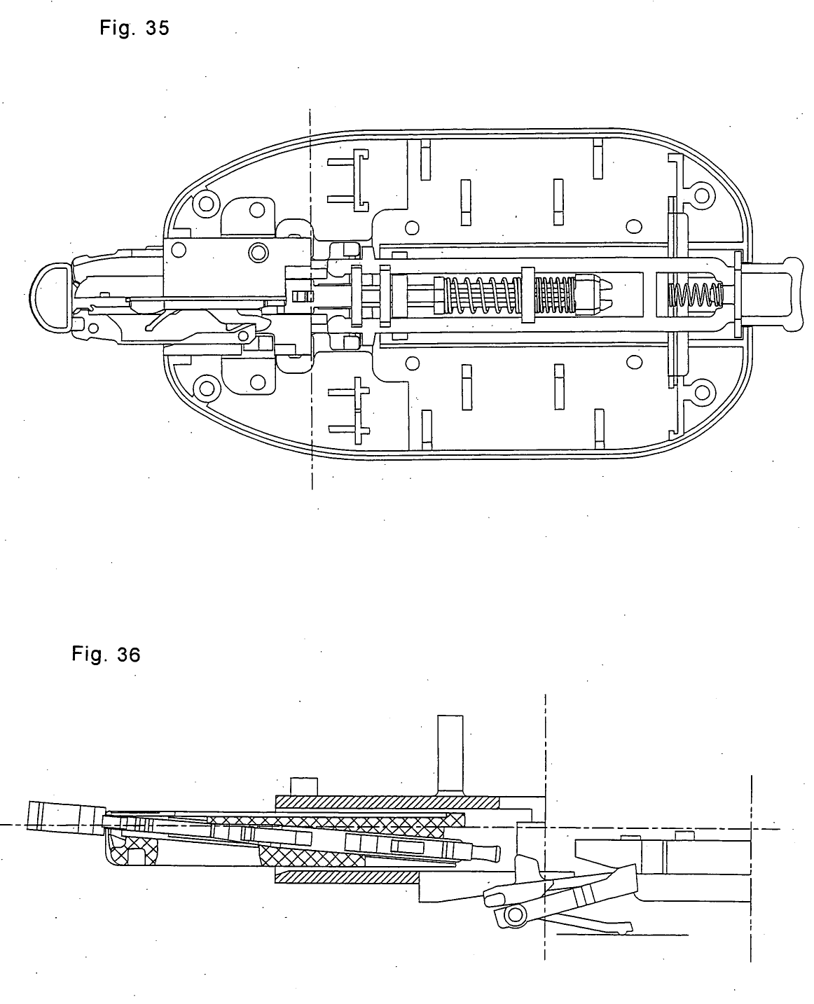

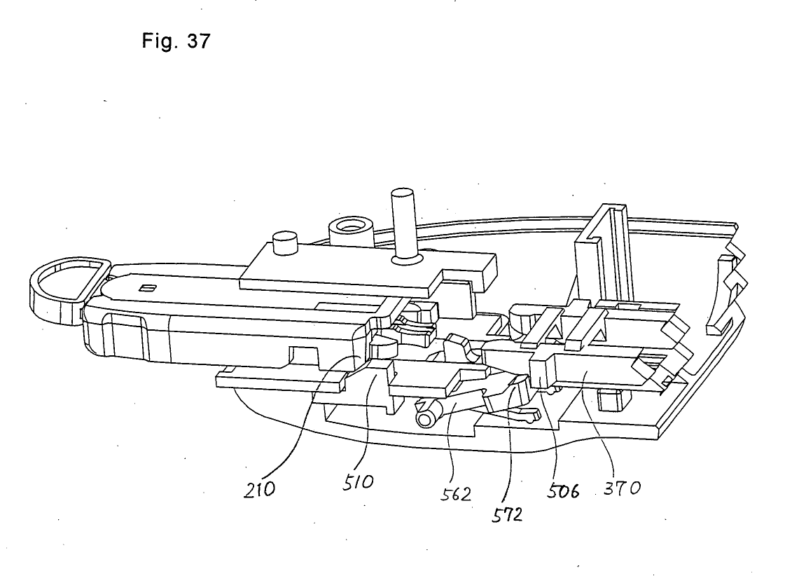

FIGS. 33 and 34 show the state prior to loading with the cassette in the plan view and the side cross-sectional view respectively. FIGS. 35 and 36 show the state wherein in the process of inserting the cassette into the connector, the rear end 210 of the cassette abuts against the engaging portion 510 which protrudes upward through the opening of the connector. In FIG. 37, for the purpose of readily understanding, a whole of the near side surface and a near side half of the upper surface of the connector 374 are cut away and also a near side half of the housing lower half is cut away, and such cutting away state would be readily understood. In such state, the leading end 362 of the ejecting member is located above the front portions of the oblique portions 562 of the stopper, so that the oblique portions have been not so much pressed downward. As a result, the level of the projections 572 provided on the rear ends of the oblique portions is not sufficiently lower than the level of the being locked portions 506 of the trigger member, which therefore become abutting against the projections 572 when the trigger member is moved forward, so that the trigger member cannot move forward any more (see FIG. 37).

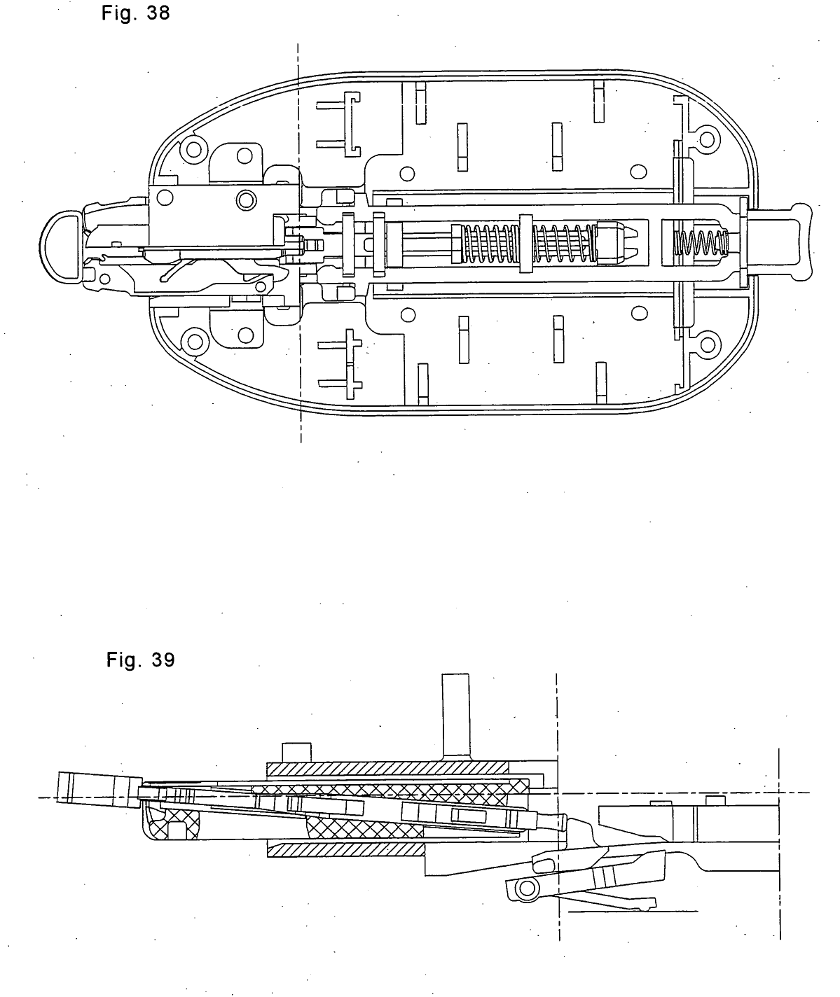

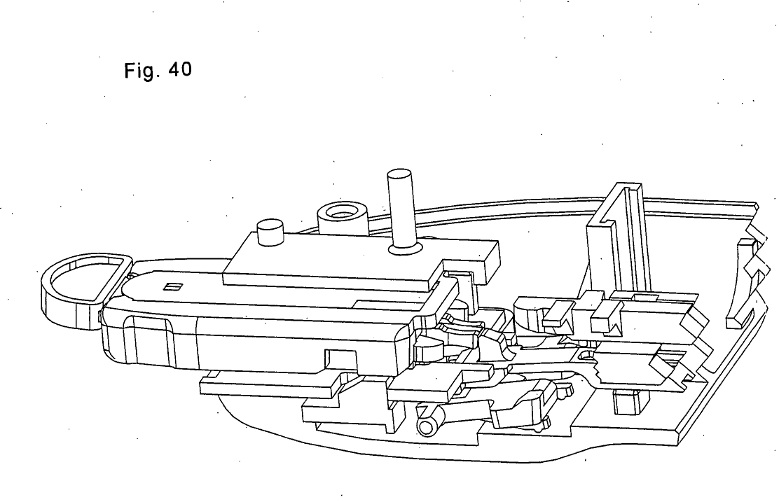

FIGS. 38 and 39 show the state wherein in the process of inserting the cassette into the connector, the cassette is further inserted from the state shown in FIGS. 35 and 36, and the rear end 142 of the lancet moves the ejecting member backward regardless of the force applied to the ejecting member by the ejection spring while the lancet rear end 142 abuts against the projection provided on the leading end of the ejecting member. It is noted that FIG. 40 shows the state wherein the engaging portion 510 is pressed downward when the cassette is further moved backward from the state shown in FIGS. 35 and 36. Thus, in the state shown in FIGS. 38 and 39, the ejection spring is further compressed from the state shown in FIGS. 35 and 36. By moving backward of the ejecting member, the pressing portion 363 formed on the lower surface of the ejecting member presses the oblique portions 562 of the stopper downward.

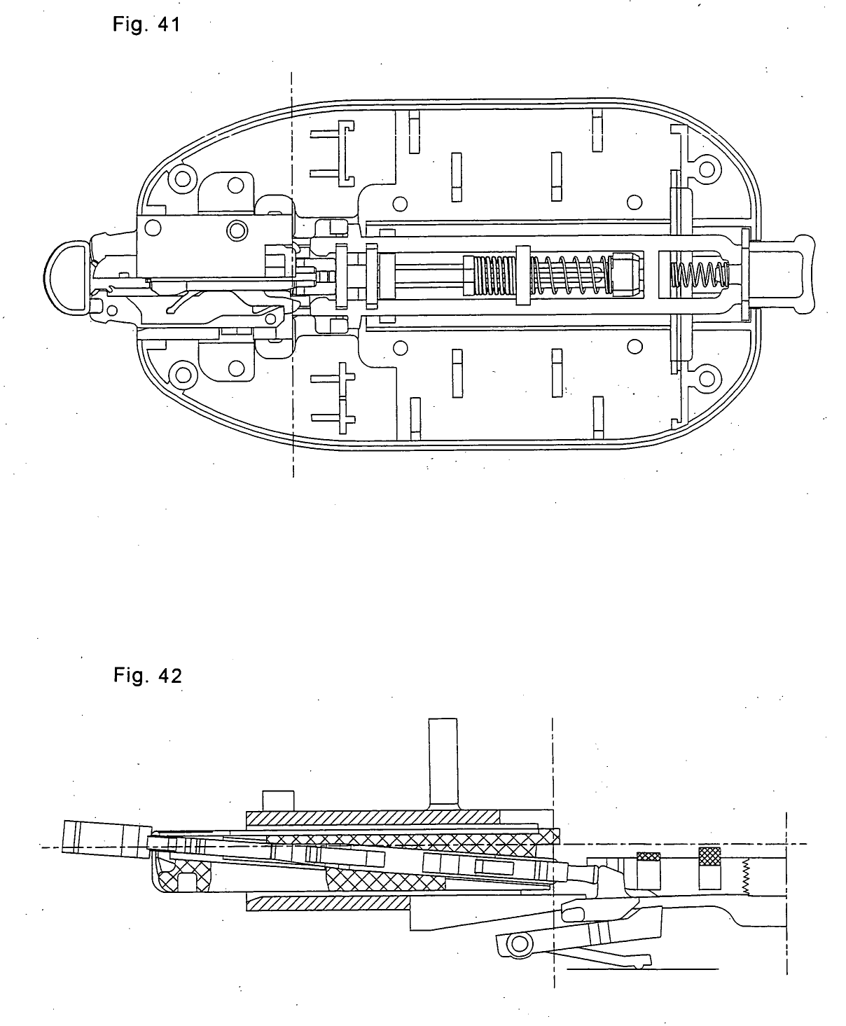

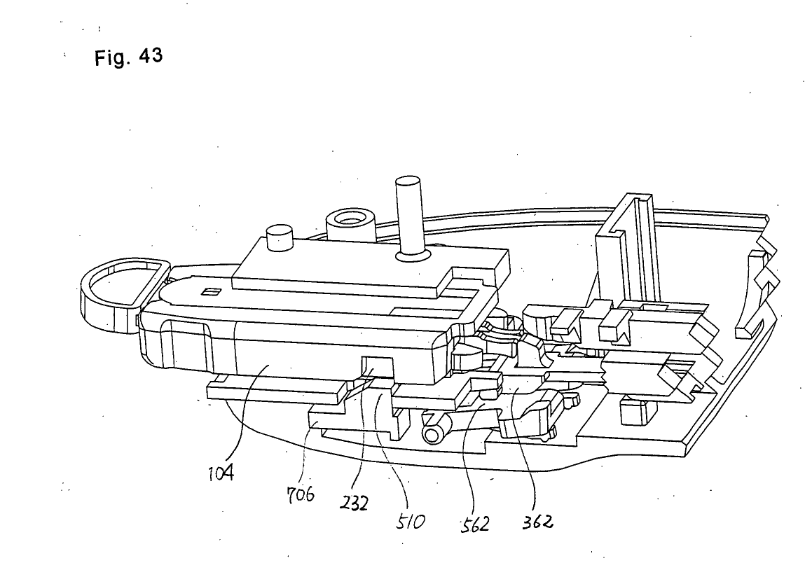

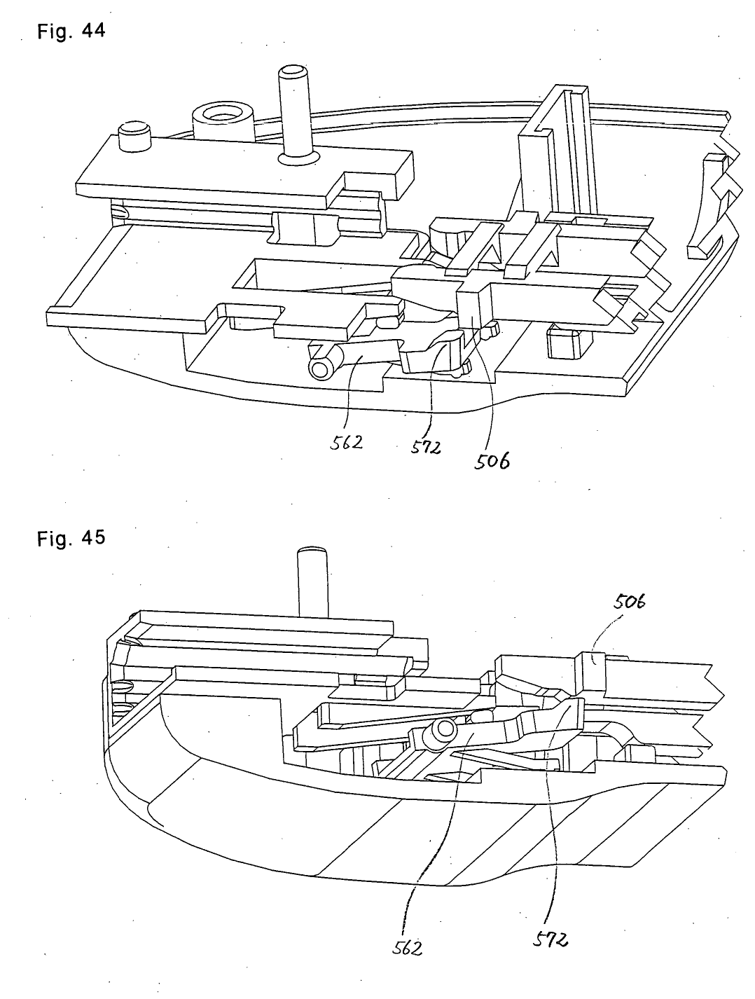

FIGS. 41 and 42 show the state wherein in the process of inserting the cassette into the connector, the cassette is further inserted from the state shown in FIGS. 38 and 39, and the engaging portions 510 are just prior to fitting into the concave portions 232 provided on the lower edge beside the lancet. In this state, the leading end 362 of the ejecting member sufficiently presses the oblique portions 562 of the stopper, so that the level of the projections 572 provided on the rear ends of the oblique portions is sufficiently lower than the level of the being locked portions 506 of the trigger member. Therefore, when the trigger member 370 is moved forward, it can proceed while its being locked portions 506 do not abut against the projections 572 (see FIGS. 44 and 45). It is noted that FIG. 44 shows the state of FIG. 43 with omitting the cassette and the engaging portion holding member 706. FIG. 45 shows the state of FIG. 44 when viewing it from its lower side.

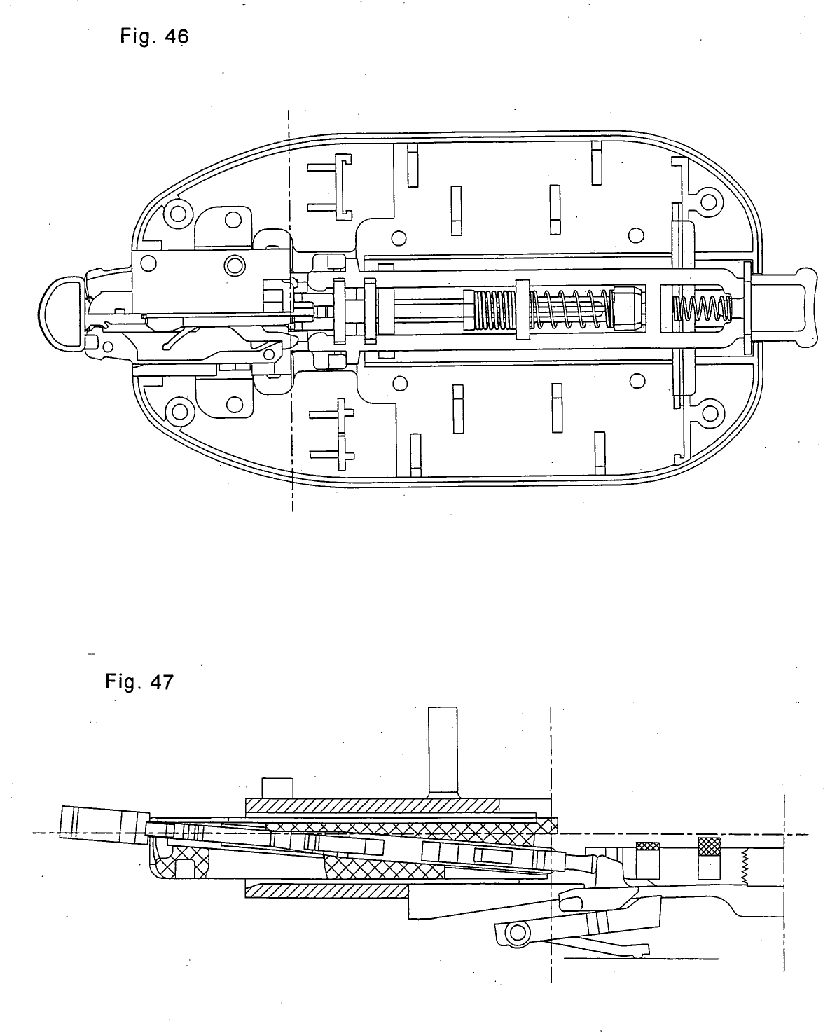

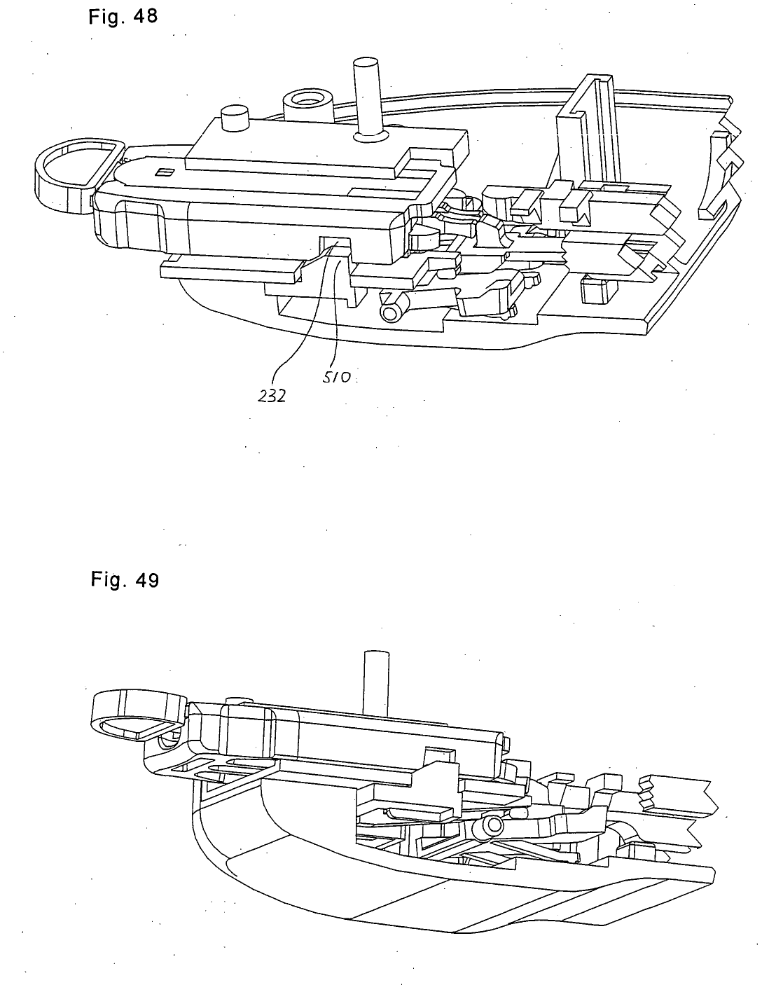

FIGS. 46 and 47 show the state which is just after the state shown in FIGS. 41 and 42 in the process of inserting the cassette into the connector. In the state of FIGS. 46 and 47, loading the ejecting device with the cassette has been completed after further insertion of the cassette so as to have the engaging portions 510 fitted into the concave portion 232 provided on the lower edge beside the lancet (see FIGS. 48 and 49).

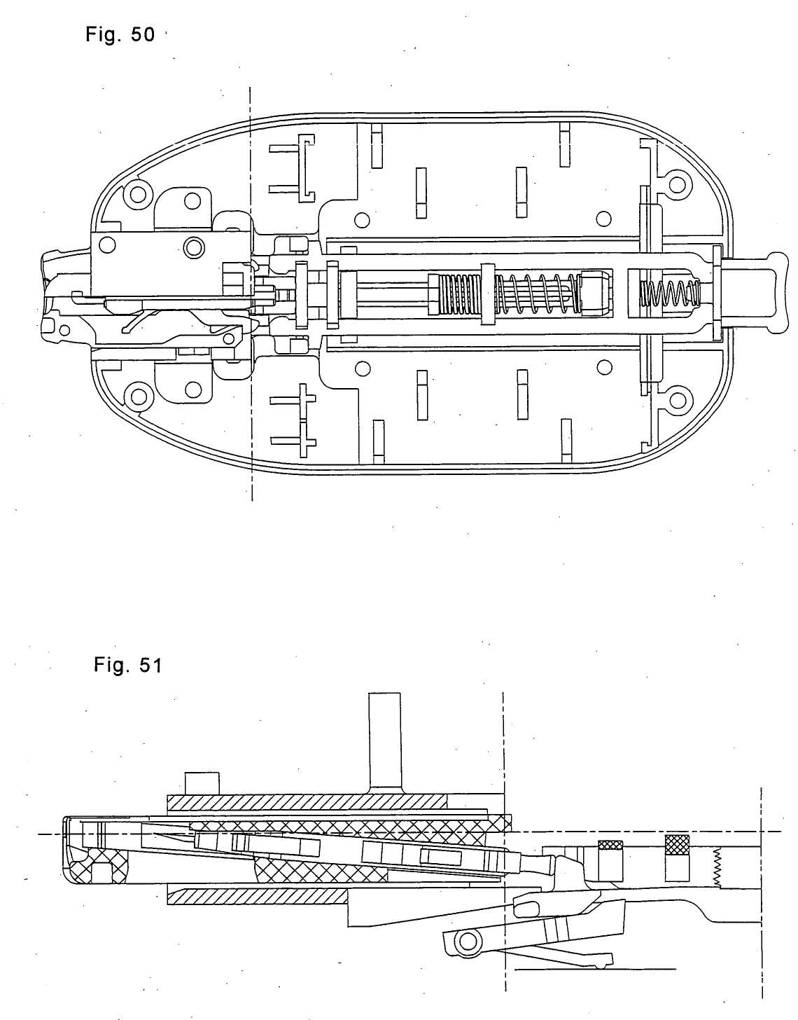

FIGS. 50 and 51 show the state wherein the tip portion 116 of the pricking member has been exposed by pulling the tab portion 150 so as to break the weakened portion 112 after the state of FIGS. 46 and 47.

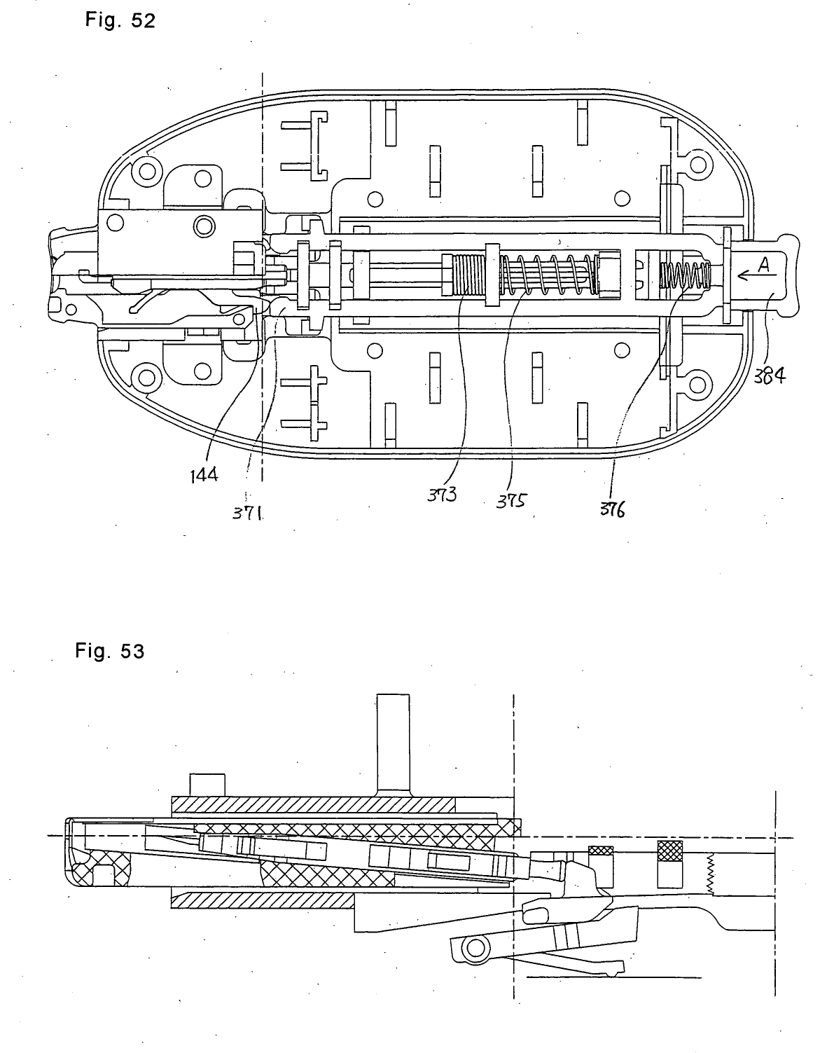

FIGS. 52 and 53 show the state wherein the rear end 384 of the trigger member is moved forward as shown with the arrow A so that the leading ends 371 of the trigger member abut against the lancet hook portions 144. When the trigger member 370 is further moved forward from the shown state, the leading ends 371 abutting against the lancet hook portions 144 deforms the hook portions to toward the inside thereof. As shown, the both leading ends 371 of the trigger member 370 define forms such that a distance between the both leading ends 371 of the trigger member 370 becomes longer toward their fronts, and the trigger member is made of a material which is more rigid than that of the lancet. Therefore, the lancet hook portions 144 are forced to deform while remaining between the two leading ends 371 of the trigger member. As a result of the deformation of the lancet hook portions 144, it becomes impossible for them to keep their engagement states with the outside of the container end 210.

It is noted that in the state of the FIGS. 52 and 53, the ejection spring 373 is further compressed (secondary compression) while an extent of the compression of the return spring 375 is relaxed when compared with the state of FIGS. 50 and 51. Further, an extent of the compression of the return spring 376 of the trigger member is increased.

FIGS. 54 and 55 show the state wherein the trigger member 370 is further moved forward from the state of FIGS. 52 and 53. When it becomes impossible for the lancet hook portions 144 to keep their engagement states with the outside of the rear end 210 of the container in FIGS. 52 and 53, the lancet 100 is intensely moved forward by the leading end 362 of the ejecting member since the ejection spring 373 is applying a force to the lancet rear end 142. FIGS. 54 and 55 show the state at the beginning moment of thus being moved forward.

FIGS. 56 and 57 show the state wherein the lancet 100 is further moved forward after the state of FIGS. 54 and 55, ands the end of the retracting portions 148 hit the collision portions 205 in the container. FIG. 59 shows the state wherein the lancet 100 is further moved after the state of FIGS. 56 and 57, and the retracting portions 148 have been bent.

From FIGS. 52 and 53 through FIGS. 54 and 55 and FIGS. 56 and 57 to FIGS. 58 and 59, the area where the lancet rear end 142 abuts against the protruding portion 364 provided on the leading end of the ejecting member gradually decreases toward discontinuation of the abutment relationship between them. This is because the moving direction of the lancet rear end 142 is obliquely upward and forward while the moving direction of the leading end 362 of the ejecting member is obliquely downward and forward relative to such moving direction of the lancet rear end. Such movement of the leading end 362 of the ejecting member 360 is ensured by the guide portion(s) 550 provided on the outside of the bottom surface of the connector.

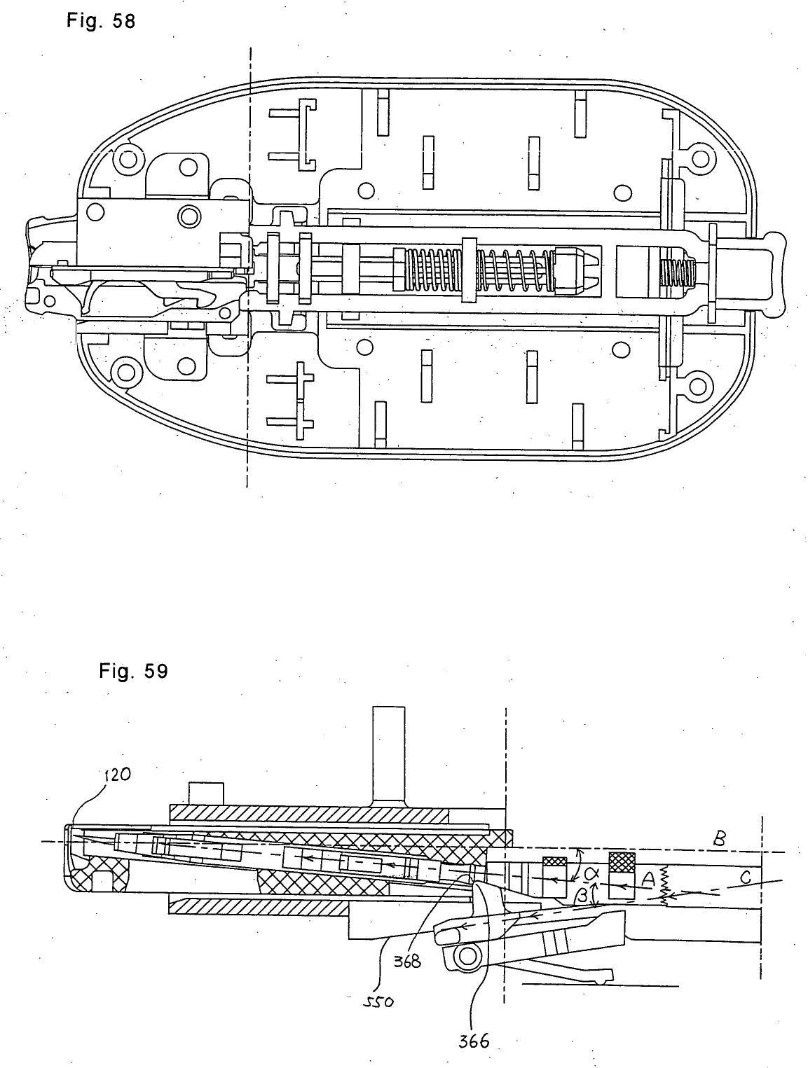

FIGS. 58 and 59 show the state wherein the abutment relationship is just before its discontinuation. As shown, an angle which is formed by the moving direction of the lancet (the arrow A) and the horizontal plane B corresponds to the angle α, and an angle which is formed by the moving direction of the lancet (the arrow A) and the moving direction of the leading end (the arrow C) corresponds to the angle β.

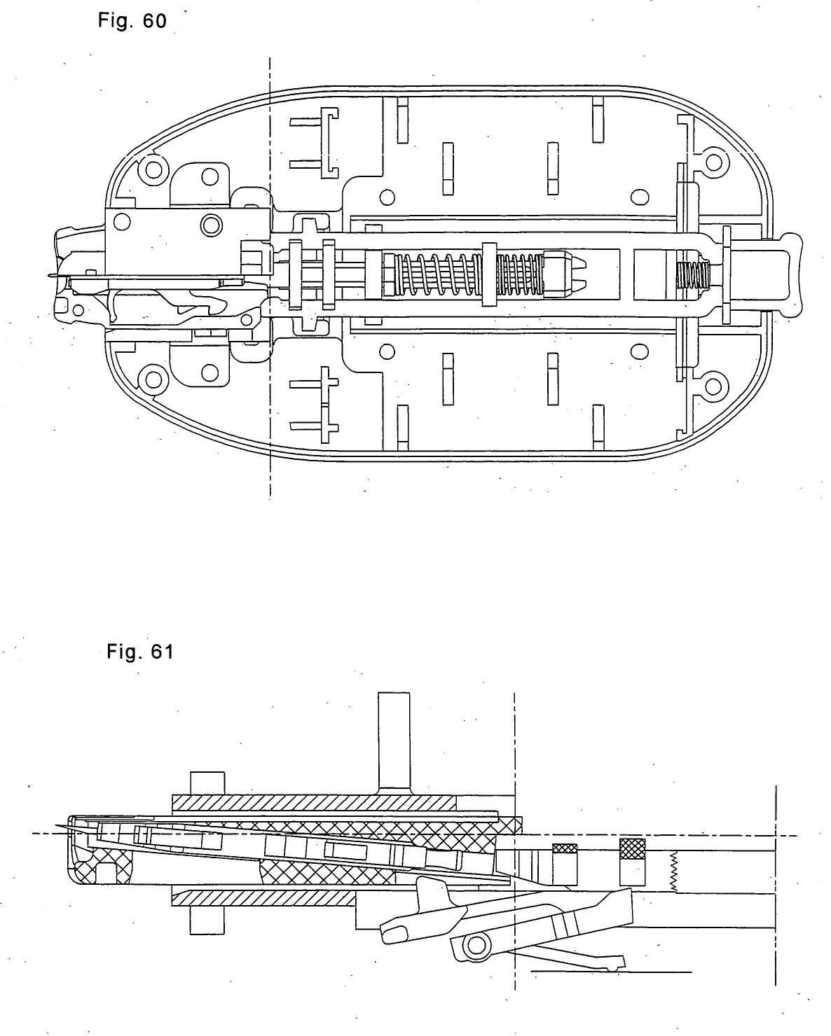

FIGS. 60 and 61 show the state after the abutment relationship between the lancet rear end 142 and the projection 364 provided on the leading end of the ejecting member is discontinued. Even though such abutment relationship is discontinued, the lancet further proceeds by means of its inertial force, so that the tip portion 116 of the pricking member is exposed outward from the opening 208 at the front end of the container 102 so as to prick the predetermined portion (not shown) which is pressed against the opening. It is noted that the retracting portions 148 have further bent from the stated shown in FIG. 59. In such state, the leading end 362 of the ejecting member 360, and thus the pressing portion 363 provided thereon has been sufficiently moved forward, so that the stopper 372 has elastically returned to its substantially original form.

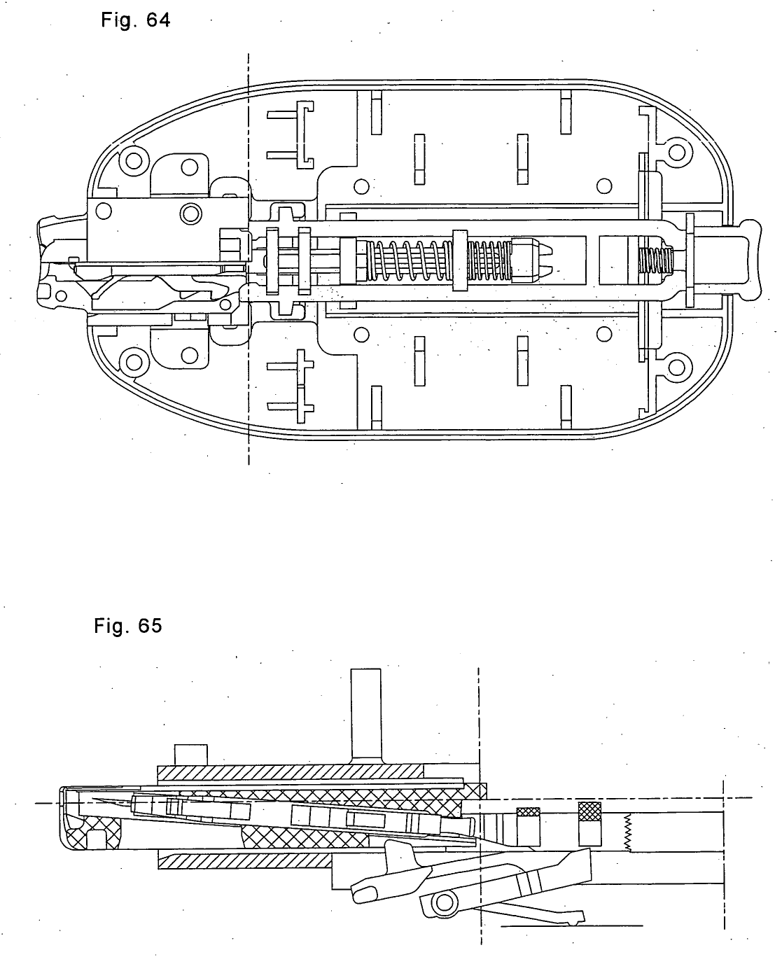

It is noted that the period after the discontinuation of the lancet hook portions by moving the trigger member 370 forward up to pricking is very short (for example from 6 milli-seconds to 10 milli-seconds), and the change from the state of FIG. 52 up to the state of FIG. 63 (or FIG. 65 referred to later) occurs instantaneously. Therefore, when the force having been applied to the rear end 384 of the trigger member 370 is released, the state shown in FIGS. 60 and 61 (or FIGS. 64 and 65 referred to later) has already come.

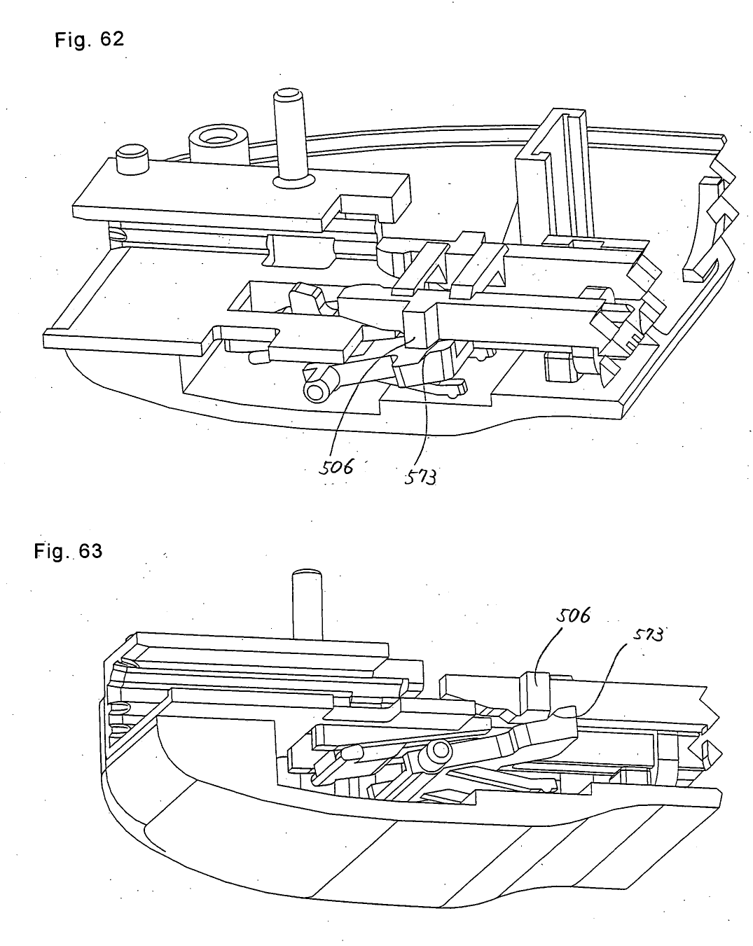

In such state, the topmost level of the protruding portion 572 provided on the oblique portion 562 is higher than the bottom most level of the being locked portion 506 of the trigger member 370, and the being locked portion 506 of the trigger member 370 which has advanced is present in front of the protruding portion 572 of the oblique portion (see FIGS. 62 and 63). Thereafter, the force applied to the rear end 384 of the trigger member 370 is released, and the trigger member 370 is moved backward by means of the function of the return spring 376 to revert to its original form. In order that the moving backward of the trigger member is not prevented by abutting of the being locked portion against the protruding portion 527, the protruding portion has an oblique surface 573. The being locked portion 506 which is moving backward can pass over the oblique surface 573 and move to the rear of the protruding portion 572.

FIGS. 64 and 65 show the state wherein the lancet has been moved back by the return of the retracting portions 148 to their original forms after pricking. In the shown state, trigger member 370 still has been in the condition of being pressed forward.

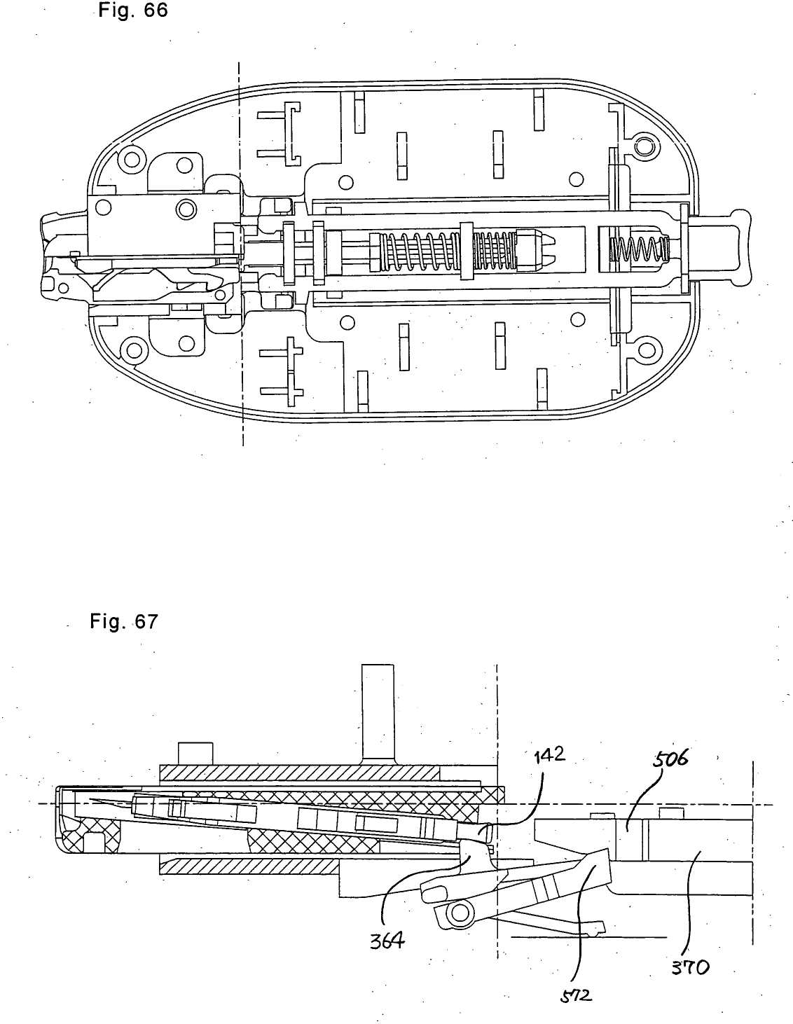

FIGS. 66 and 67 show the state where the force which presses the trigger member forward has been released, so that the trigger member 370 has been moved back, and also the ejecting member 360 has been moved back which member is connected to the trigger member 370 by means of the partition member 384. That is, the leading end 362 of the ejecting member is located below the lancet rear end 142.

Therefore, even when the ejecting device is loaded with a cassette which contains a used lancet, the state shown in FIGS. 66 and 67 comes, so that the lancet rear end 142 does not abut against the protruding portion provide on the leading end of the ejecting member. Thus, when loaded with the already used lancet erroneously, it is substantially impossible to prick using such lancet. It is noted that when loaded erroneously, it may be possible that the lancet rear end 142 is present in the vicinity of or adjacently to the protruding portion provided on the leading end of the ejecting member as shown in FIGS. 68 and 69. Even with such possibility, when the rear end 142 of the lancet and protruding portion are formed to have round portions respectively, the rear end 142 slides on and not engages with the protruding portion 364 so that the state shown in FIGS. 66 and 67 is achieved.

Further, as clearly seen from FIG. 67, once the trigger member 370 has been moved back, the topmost level of the protruding portion 572 provided on the rear end of the oblique portion is higher than the bottom most level of the being locked portion 506 of the trigger member 370, so that the rear end and the being locked portion can be abutted against each other, so that the forward movement of the trigger member is prevented.

Thereafter, moving the discharge button 710 forward so as to remove the protruding portions 510 out from the concave portions 232 of the container and the engagement relationship between the cassette and the ejection device is discontinued. Then, the cassette is pulled out of the connector of the ejecting device so as to discharge the cassette.

As explained above, the cassette according to the present invention may comprise the sensor element. Such sensor element may be arranged on the outer surface 207 or the inner surface 201 of the upper half 202 of the assembled cassette. The arrangement may be carried out in any appropriate manner. It is noted that the terms “outer surface” and “inner surface” mean that a surface which is located outside the upper half shown in FIG. 8 (that is, the surface which is present on the back side of the upper half 202 in FIG. 8) and a surface which is located inside the upper half shown in FIG. 8 (that is, the surface which is present on the near side of the upper half 202 in FIG. 8) respectively.

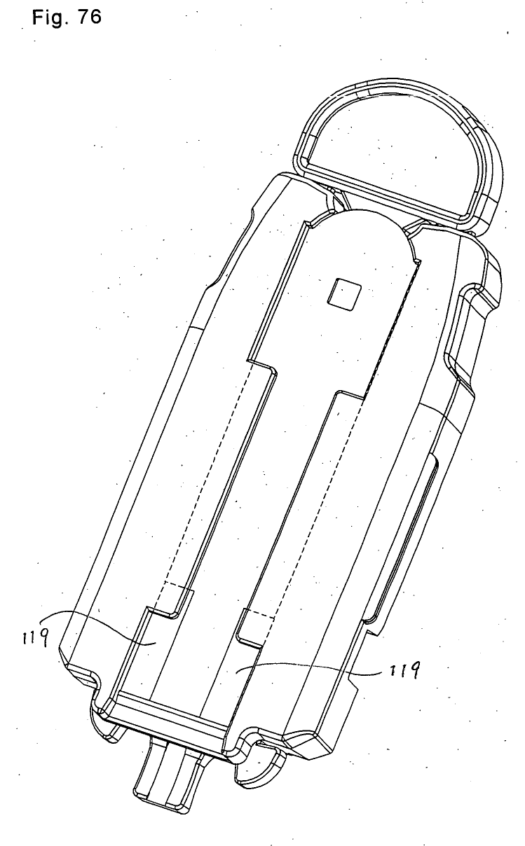

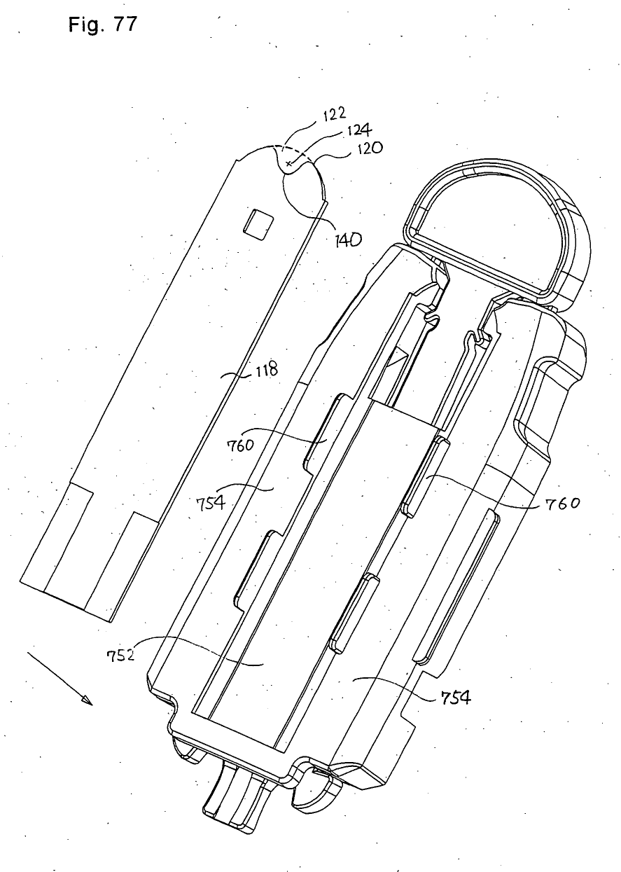

In one embodiment wherein the sensor element is arranged on the outer surface, the outer surface 207 is formed such that it has a base surface 752 as well as side surfaces 754 beside the base surface, which define a concave portion 750 for receiving the sensor element 118. A level difference between the base surface 752 and the side surfaces 754 is preferably at least the same as a thickness of the sensor element. At least a portion of an edge of the side surface 754 which edge is closer to the base surface 752 has an extension 758 which extends above the base surface. The sensor element is put between the base surface 752 and the extensions 758, so that falling off of the sensor element 118 from the outer surface of the upper half is prevented. Therefore, it is preferable that the gap between the base surface 752 and the extension 758 is substantially the same as the thickness of the sensor element, so that the sensor element 118 is firmly held on the outer surface. In the shown embodiment, the sensor element 118 may be inserted from the container end while sliding it. The state after the insertion is shown in FIG. 76. The rear end 119 of the sensor element is preferably exposed as shown.

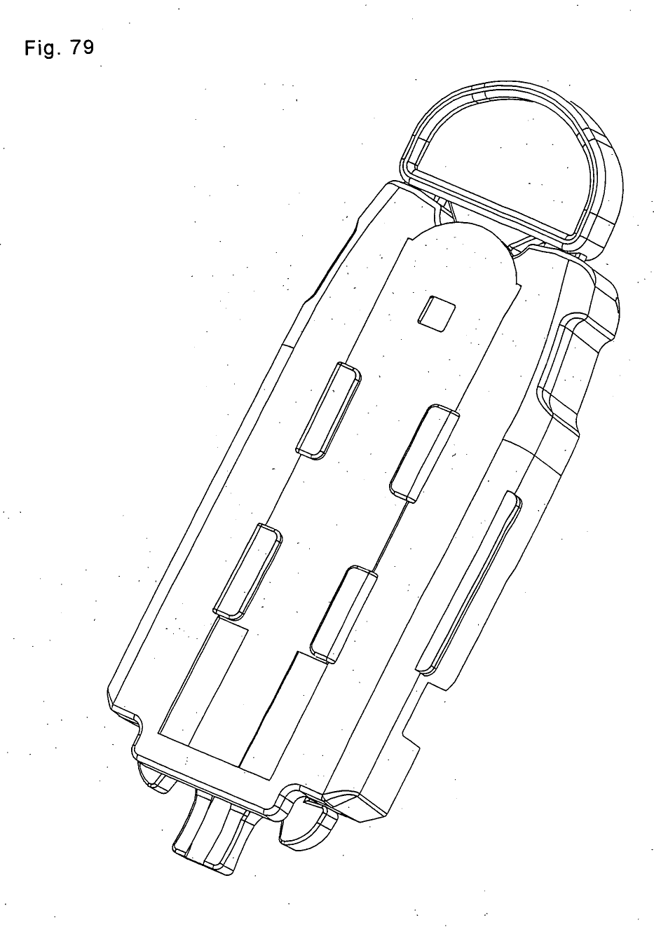

In other embodiment, the sensor element may be placed from the above of the outer surface as shown in FIG. 77. In the shown embodiment, at least a portion of the edge of the side surface 754 which edge is closer to the base surface 752 has a protruding portion(s) 760 along the edge. The sensor element 118 is placed on the base surface 752 as shown with the arrow so as to achieve the state as shown in FIG. 78. Then, the protruding portion(s) 760 is heated by means of ultrasonic so as to fuse the resin which forms the upper half and spread the fused resin onto a portion of the edge portions of the sensor element followed by solidifying the spread resin, so that the sensor element is placed on the upper surface as shown in FIG. 79.

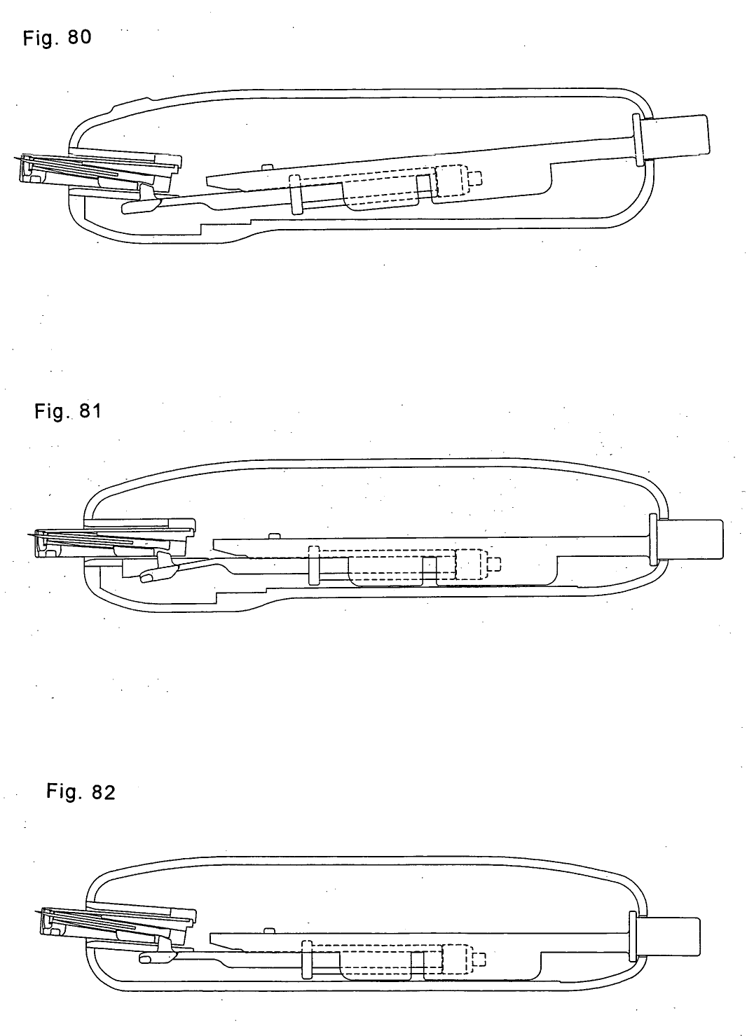

As explained above, in the lancet assembly according to the present invention, the pricking direction of the lancet is not parallel to the moving direction of the ejecting member, and these directions form the angle β as shown in FIG. 59. When those two directions form thus angle, the leading end of the ejecting member 360 is folded or bent with respect to its remaining rear potion for example as shown in FIG. 61. The ejecting member 360 is made straight as a whole as shown in FIG. 32. However, it is preferable that the ejecting member is made of a resin and a portion thereof behind its leading end is made thin so that the ejecting member is folded while the leading end follows the guide portion 550 as shown in FIG. 61. Thus foldable embodiment of the ejecting member is shown in FIG. 81, but it is not necessarily required to be foldable. For example, it is possible for the ejecting member as a whole to be straight as shown in FIG. 80. In this case, since the moving direction of the trigger member is obliquely downward relative to the horizontal plane, a user may feel uncomfortable. To the contrary, when the moving direction of the trigger is set horizontal, the direction along which the cassette is inserted into the connector is upward relative to the horizontal plane, and in this case also, the user may feel uncomfortable.

### EXAMPLES

Pricking Member

Needles made of a stainless steel (SUS 304) which are conventionally used for lancets were used as the pricking members 106. The needles had a diameter of 0.4 mm.

Lancet

Lancets 100 as shown in FIG. 15 were produced. The lancet body 108 and the shield member 110 were produced by injection molding of LLDPE resin (manufactured by Japan Polyolefin Corporation) while inserting the pricking member (made of the stainless steel SUS 304) in the mold. The pricking member was located such that the tip portion 116 having a length of 1.9 mm was exposed from the lancet body 108. It is noted that the lancet was designed to be a form which was linear symmetry with respect to the pricking member as an axis in the horizontal plane, and also which had substantially no difference between its top side and under side.

The connection between the shield member 110 and the lancet body 108 was made to be the weakened portion 112 as the notches as shown in the drawings, and designed such that by pulling away (that is, pulling off) so as to separate toward opposing directions at the connection, the breakage of the connection happens. Concretely, a thickness (that is, a thickness of the resin around the pricking member) at the notch portion was set at 0.4 mm such that the connection was broken by a force which would be applied when finger tips hold the shield member 110 and the container 102 and separate them away (for example, a force of about 0.7 kg to 0.85 kg). The completed lancet had an overall size of 35.2 mm (length)×12.4 mm (width)×2.5 mm (thickness).

The preforms 220 for the container as shown in FIG. 8 ware molded. Considering that the container has the hinge portion 218, the container was produced by injection molding of a compound of G-P polystyrene (manufactured by Toyo Polystyrene Co., Ltd.) and styrene-butadiene copolymer resin (manufactured by Phillips). It is possible to use a polypropylene (PP) resin, a polyethylene (PE) resin, a PE-PP copolymer resin or the like, and a polystyrene is preferable since its contraction coefficient upon molding is small so that a precise molded article can be produced.

It is noted that the container was designed such that the angel α is 4.5° in order that the lancet 100 moves obliquely relative to the horizontal plane 136.

Assembling of Lancet Cassette

After the molding of the container, the mold was opened such that the preform extended over the horizontal plane, and the lancet 100 was placed on the lower half 204 of the container such that the hook portions 144 were adjacent to the outside of the rear end 210 of the container, the lancet rear end 142 protruded from the opening 212 at the rear end of the container, and the other portions of the lancet were located within the container. Then, the container was folded around the hinge portion 218 so as to stack the upper half 202 on the lower half 204 and projections were press fitted into small circular holes, whereby the lancet cassette as shown in FIG. 12 was obtained.

Then, the sensor element 118 was placed in the concave portion (that is, on the base surface 752) defined by the step portions (or shoulder portions) formed on the outer surface 207 of the upper half of the lancet cassette. It is noted that the protruding portions 760 formed on the step portions were deformed by an ultrasonic welding machine so as to prevent the sensor element from falling off. Thus placed sensor element was located in the concave portion as predetermined so that it was not moved even when tried to move it by a finger.

Since the protruding portions 154 which extended backward from the shield portion 150 remained between the upper half 202 and the lower half 204 immediately inside from the opening at the front end of the container, the unity of the shield member 110 and the container 102 was increased. As a result, the operation to fit the cassette into the ejecting device was possible without any problem while holding the shield portion 150 itself without holding the cassette. It was of course possible without any problem to fit the cassette into the connector while holding the cassette after pulling off the shield member 110 from the cassette.

Housing of Lancet Ejecting Device

The lancet ejecting device as shown in FIG. 6 was produced. Concretely, three dimensional digital information of the shapes and the sizes of the upper housing half 702 and the lower housing half 704 were transferred to an NC fraise, and the housing halves were prepared by cutting ABS resin blocks. It is noted that the housing halves can be produced by injection molding for the purpose of the mass production thereof.

Similarly, the followings were produced from resin blocks: the cover member 377 (made of an ABS resin), the stopper 372 (made of a polyacetal resin), the connector 374 (made of a polycarbonate resin), the trigger member 370 (made of a polycarbonate resin), the pressing member 714 (made of a polyacetal resin), the engaging portion holding member 706 (made of a polyacetal resin), the elastic member 712 (made of a polyacetal resin), the discharge button 710 (made of a polyacetal resin), the partition member for the ejecting member 384 (made of an ABS resin) and the guide 398 (made of an ABS resin). The guide portions 550 were formed on the connector such that the angle β formed by the lancet ejecting member described later and the lancet moving direction was 13.2°.

Lancet Ejecting Member

Similarly to the above preparation for the housing except that a polyacetal resin in place of the ABS was used, the ejecting member 360, the stopper 372 and the flange member 388 were prepared by cutting the resin blocks.

It is noted that the engaging portion holding member 706, and the cassette discharge mechanism 710, 712 and 714 were also prepared similarly. As the elastic member 512, a foam made of a silicone foam was used.

Metal Springs

The ejection spring 373 had a capacity to provide a force of 565 g, and the return spring 375 had a capacity to provide a force of 270 g. The return spring 376 for the trigger member had a capacity to provide a force of 50 g. All of the springs were made of the stainless steel (SUS 304).

The lancet ejecting device 400 as shown in FIG. 6 was obtained by assembling the above produced members or pats.

Ejection Tests

The cassette 104 of which shield member 110 has not been pulled off was inserted into the connector 374 of the lancet ejecting device 374 while the surface on which the sensor element was placed was upward. When the cassette was inserted over a distance of about 10 mm from the opening 522 of the connector, the rear end 142 of the lancet protruding from the rear end of the cassette abutted against the protruding portion 364 provided on the leading end of the lancet ejecting member.

Then, when the cassette was further inserted, the engaging portions 510 of the engaging portion holding member fitted into the concave portions 232 of the cassette, so that the cassette was engaged within the connector as predetermined. Such engaging state means that the ejection spring has been primarily compressed as predetermined. Thereafter, the shield member of the lancet was pulled off so as to finish the preparation for pricking. Upon the insertion of the cassette, the trigger member 370 was not moved at all.

Next, a finger tip was pressed against the opening 208 of the cassette at its front end, and then the rear end 384 of the trigger member was pressed. While the trigger member was moved forward over a distance of about 5 mm, the ejection spring 373 was secondarily compressed, and at the final stage of such compression, the leading end 371 of the trigger member released the engagement relationship of the hook portions 144 with the outside of the cassette, so that the lancet 100 was ejected.

As explained above, with the ejecting device according to the present invention, even though only the secondary compression is carried out by pressing the trigger member when the lancet is ejected, the energy used for the ejection is a sum of energies of the primary compression and the secondary compression. Therefore, the device according to the present invention gives a user of the device an impression that only a little push-in of the trigger member allows the ejection of the lancet, that is, the ejection can be easily done like pressing a button.

The lancet in the cassette was automatically moved back by means of the force of the curved wings 148 to elastically revert back to their own forms (i.e. a spring like force) after pricking, so that the tip portion of the pricking member was located within the container 116, which resulted in the state wherein the needle end did not protrude outward from the front end of the container.

An amount of blood bled and a portion thereof deposited to the blood intake port in a spot form which was located at the end of the sensor element, and the deposited blood was taken into the inside of the element. Since the front end of the cassette was designated to be located about 8 mm ahead of the front end of the connector of the ejecting device, it was estimated that the possibility that the bled blood deposited to the ejecting device was substantially zero.

After pricking followed by removing the finger tip from the trigger member so as to return the trigger member to its original form, forward pressing of the trigger member was tried. The being locked portions 506 provided on the leading ends of the trigger member abutted against the protruding portions 572 provided on the oblique portions of the stopper, so that forward pressing of the trigger member was impossible. Thus, there was no influence at all due to such trial on the lancet retracted in to the cassette. After discharging the spent cassette, the trigger member was similarly pressed, but the above explained locked state was caused as well, so that the trigger member was not moved.

Then, a used cassette was inserted connector again and engaged therein by means of the engaging portions 510. As to the used cassette, a whole of the lancet is contained in the container, and the rear end 142 of the lancet is also located substantially within the container. As a result, the rear end of the lancet was not able to abut against the protruding portion provided on the leading end of the ejecting member, so that the projection member was not be able to be moved backward, and the stopper remained working. That is, even when the trigger member was tried to be moved forward, it was locked so that the trigger member was not able to be pressed forward.

After discharging the used cassette, a cassette which contained a used lancet was inserted into the connector. In the process of the insertion, it was grasped through a response to fingers through the cassette that the rear end of the lancet abutted against the protruding portion of the leading end of the ejecting member, so that the ejection spring was compressed to be in the cocked state, wherein pressing the trigger forward was possible when it was tried. That is, through the insertion of the cassette, the rear end of the lancet moves the leading end of the ejecting member backward so that the oblique surfaces of the safety stopper which lock the trigger member are pressed downward by the cam-like portion provided on the lower side of the leading end of the ejecting member, whereby even when the trigger member is moved forward, the trigger member does not abut against the being locked portions. Therefore, in such state, the ejection was possible by moving the trigger member forward.

The used cassette was observed. The lancet within the container was automatically retracted by means of the spring-like effect of the retracting portions provided on the lancet body as the wings, and the frontmost end of the pricking member was located 2 mm behind the opening of the front end of the container, and the rear end of the lancet was inside the opening of the rear end of the container. This state can be said to be the lancet being substantially shielded by the container. In such state, it is prevented as much as possible that an accidental force is applied to the lancet in the container so that the tip portion of the pricking member is exposed outward from the opening of the container at its front end.

Pricking Strength Test

Pricking properties of the cassette and the lancet ejecting device were checked. For the comparison, Multi-Lancet II (commercially available from Arkray, needle diameter: 0.4 mm) which was able to set five different pricking depths was used at the pricking depth of “3”. Stacking twenty sheets of copy paper each having a thickness of 0.075 mm. The stacked sheets were regarded as a skin to be pricked as predetermined by the lancet, and the number of sheets which were pricked was counted.

With the lancet assembly according to the present invention, the number was 7.4 on average, while with Multi-Lancet, the number was 6.9 on average. From these results, it is seen that the lancet assembly according to the present invention has the same pricking properties as those of the commercially available lancet assembly.

It is noted that the present invention includes the following modes (or embodiments):

Mode 1

A lancet cassette comprising a lancet which includes a pricking member for piercing a predetermined portion as well as a container which has a space therein where the lancet moves, characterized in that
the lancet cassette is capable of mounting a thin sensor element thereon, and
a moving direction of the pricking member of the lancet intersects with an extending plane of the sensor element in the vicinity of a front end of the sensor element.
   Mode 2

The lancet cassette according to Mode 1 wherein the vicinity of the front end of the sensor element is any one of an immediate front from the front end of the sensor element, substantially the front end of the sensor element or an immediate rear of the front end of the sensor element.

Mode 3

The lancet cassette according to Mode 1 or 2 wherein the direction of the pricking member of the lancet intersects with the extending plane of the sensor element so as to form an angle between 3.5° to 7°.

Mode 4

The lancet cassette according to any one of Modes 1 to 3 wherein the lancet further comprises a lancet body and a shielding member which are connected through weakened portions with each other, and the pricking member is encapsulated with the lancet body and a shielding member except a portion which is adjacent to the weakened portions.

Mode 5

The lancet cassette according to any one of Modes 1 to 4 wherein the lancet body comprises a lancet rear end, lancet hook portions located on either side of the lancet rear end and a lancet pull-out preventive portion located in front of each of the lancet hook portions,
the lancet rear end protrudes outward from an opening at a rear end of the container,
each of the lancet hook portions is located outside and adjacently to the rear end of the container and engages therewith,
the lancet pull-out preventive portion is located inside and adjacently to the rear end of the container, and
the lancet is housed in the container.
   Mode 6

The lancet cassette according to any one of Modes 1 to 5 wherein the lancet further comprises a retracting portion.

Mode 7

The lancet cassette according to any one of Modes 1 to 6 wherein the container comprises the sensor element which is mounted thereon.

Mode 8

The lancet cassette according to any one of Modes 1 to 7 wherein the container is composed of an upper half and a lower half which are connected together with a hinge portion.

Mode 9

A lancet assembly comprises (1) the lancet cassette according to any one of Modes 1 to 8 and (2) a lancet ejecting device which comprises a lancet ejecting member and ejects the lancet.

Mode 10

The lancet assembly according to Mode 9 wherein by fitting a rear portion of the lancet cassette into the lancet ejecting device through an opening at a front end of the lancet ejecting device, the rear end of the lancet abuts against a leading end of the lancet ejecting member, then the container is moved back against a forward force acting on the lancet ejecting member so that the lancet ejecting member is moved back, and when the lancet cassette is inserted into the lancet ejecting device over a predetermined length from the opening of the front end of the lancet ejecting device, so that the lancet cassette is engaged with the lancet ejecting device, so that the lancet cassette is held while a forward force acting on the lancet ejecting device is acting on the rear end of the lancet.

Mode 11

The lancet assembly according to Mode 10 wherein a front end of the lancet cassette which is engaged with the lancet ejecting device protrudes from the front end of the lancet ejecting device.

Mode 12

The lancet assembly according to any one of Modes 9 to 11 wherein the lancet ejecting device comprises a lancet ejection trigger member which releases engagement of the lancet hook portions with the outside of the rear end of the container,
forward movement of the lancet ejection trigger member substantially releases the engagement of the lancet hook portions with the outside of the rear end of the container, so that the lancet ejecting member and the lancet of which rear end abuts against the lancet electing member instantaneously move forward by the forward force acting on the lancet ejecting member, so that the lancet hook portions and the lancet rear end get into the inside of the container in which they move forward, and
as a result, a tip portion of the pricking member which has been exposed from the front end of the lancet body by disconnecting the shield member from the lancet body protrudes from the opening at the front end of the container and pierces the predetermined portion.
   Mode 13

The lancet assembly according to any one of Modes 9 to 12 wherein when the lancet ejecting member and the lancet of which rear end abuts against the lancet electing member instantaneously move forward, a moving direction of the lancet is forward and obliquely upward relatively to a moving direction of the lancet ejecting member,
in the process where they moves forward and the tip portion of the pricking member pierces the predetermined portion, the engagement between the lancet ejecting member and the lancet rear end is released and then a leading end of the lancet ejecting member is located below the container.
   Mode 14

The lancet assembly according to any one of Modes 9 to 13 wherein after piercing the predetermined portion, the container receives the lancet such that the rear end of the lancet is prevented from protruding outward from the opening of the rear end of the container,
as a result, when the rear portion of the cassette which includes thus received spent lancet is engaged with the lancet ejecting device by inserting it into the lancet ejecting device through the opening at the front end of the lancet ejecting device over the predetermined length, the rear end of the lancet is located above and without abutting against the leading end of the lancet ejecting member.
   Mode 15

The lancet assembly according to any one of Modes 9 to 14 wherein the lancet ejecting member comprises a stopper which prevents forward movement of the trigger member which initiates the ejection of the lancet except when the forward force acting on the lancet ejecting member acts on the rear end of the lancet while the rear end of the lancet is held with abutting against the leading end of the lancet ejecting member,
the stopper comprises a base portion and an oblique portion which is inclined with respect to the base portion, and front ends of these two portions are connected together while rear ends thereof are separated such that when a force is applied to the oblique portion, the stopper deforms elastically so that the rear end of the oblique portion and rear end of the base portion which are separated get closer to each other and when such force is removed, the stopper elastically revert to its original form,
in the case in which the stoppers in its original form, the rear end of the oblique portion abuts against a being locked portion of the trigger member which tries to move forward so as to prevent the forward movement,
when the rear portion of the cassette is fitted into the lancet ejecting device through the opening at its front end so that the lancet ejecting member is moved back while the rear end of the lancet abuts against the leading end of the lancet ejecting member, the leading end of the lancet ejecting member moved back moves on the oblique portion of the stopper, so that the leading end of the lancet ejecting member applies a force onto the oblique portion of the stopper, whereby the stopper elastically deforms and makes the rear end of the base portion and the rear end of the oblique portion which have been separated get closer to each other, and
as a result, the trigger member which tries to move forward is able to move forward while passing over the oblique portion with the being locked portion of the trigger member not abutting against the rear end of the oblique portion.
   Mode 16

The lancet assembly according to any one of Modes 9 to 15 wherein the lancet ejecting device further comprises an electronic device which carries out a predetermined measurement with respect to an amount of blood which bleeds due to pricking.

Mode 17

A lancet ejecting device which forms, in combination with the lancet cassette according to any one of Modes 1 to 8, the lancet assembly according to any one of Modes 9 to 16.

1. A lancet cassette comprising a lancet which includes a pricking member for piercing a predetermined portion as well as a container which has a space therein where the lancet moves, characterized in that
the lancet cassette is capable of mounting a thin sensor element thereon, and
a moving direction of the pricking member of the lancet intersects with an extending plane of the sensor element in the vicinity of a front end of the sensor element.

2. The lancet cassette according to claim 1 wherein the vicinity of the front end of the sensor element is any one of an immediate front from the front end of the sensor element, substantially the front end of the sensor element or an immediate rear of the front end of the sensor element.

3. The lancet cassette according to claim 1 wherein the direction of the pricking member of the lancet intersects with the extending plane of the sensor element so as to form an angle between 3.5° to 7°.

4. The lancet cassette according to claim 1 wherein the lancet further comprises a lancet body and a shielding member which are connected through weakened portions with each other, and the pricking member is encapsulated with the lancet body and a shielding member except a portion which is adjacent to the weakened portions.

5. The lancet cassette according to claim 1 wherein the lancet body comprises a lancet rear end, lancet hook portions located on either side of the lancet rear end and a lancet pull-out preventive portion located in front of each of the lancet hook portions,
the lancet rear end protrudes outward from an opening at a rear end of the container,
each of the lancet hook portions is located outside and adjacently to the rear end of the container and engages therewith,
the lancet pull-out preventive portion is located inside and adjacently to the rear end of the container, and
the lancet is housed in the container.

6. The lancet cassette according to claim 1 wherein the lancet further comprises a retracting portion.

7. The lancet cassette according to claim 1 wherein the container comprises the sensor element which is mounted thereon.

8. The lancet cassette according to claim 1 wherein the container is composed of an upper half and a lower half which are connected together with a hinge portion.

9. A lancet assembly comprises (1) the lancet cassette according to claim 1 and (2) a lancet ejecting device which comprises a lancet ejecting member and ejects the lancet.

10. The lancet assembly according to claim 9 wherein by fitting a rear portion of the lancet cassette into the lancet ejecting device through an opening at a front end of the lancet ejecting device, the rear end of the lancet abuts against a leading end of the lancet ejecting member, then the container is moved back against a forward force acting on the lancet ejecting member so that the lancet ejecting member is moved back, and when the lancet cassette is inserted into the lancet ejecting device over a predetermined length from the opening of the front end of the lancet ejecting device, so that the lancet cassette is engaged with the lancet ejecting device, so that the lancet cassette is held while a forward force acting on the lancet ejecting device is acting on the rear end of the lancet.

11. The lancet assembly according to claim 10 wherein a front end of the lancet cassette which is engaged with the lancet ejecting device protrudes from the front end of the lancet ejecting device.

12. The lancet assembly according to claim 9 wherein the lancet ejecting device comprises a lancet ejection trigger member which releases engagement of the lancet hook portions with the outside of the rear end of the container,
forward movement of the lancet ejection trigger member substantially releases the engagement of the lancet hook portions with the outside of the rear end of the container, so that the lancet ejecting member and the lancet of which rear end abuts against the lancet electing member instantaneously move forward by the forward force acting on the lancet ejecting member, so that the lancet hook portions and the lancet rear end get into the inside of the container in which they move forward, and
as a result, a tip portion of the pricking member which has been exposed from the front end of the lancet body by disconnecting the shield member from the lancet body protrudes from the opening at the front end of the container and pierces the predetermined portion.

13. The lancet assembly according to claim 9 wherein when the lancet ejecting member and the lancet of which rear end abuts against the lancet electing member instantaneously move forward, a moving direction of the lancet is forward and obliquely upward relatively to a moving direction of the lancet ejecting member,
in the process where they moves forward and the tip portion of the pricking member pierces the predetermined portion, the engagement between the lancet ejecting member and the lancet rear end is released and then a leading end of the lancet ejecting member is located below the container.

14. The lancet assembly according to claim 9 wherein after piercing the predetermined portion, the container receives the lancet such that the rear end of the lancet is prevented from protruding outward from the opening of the rear end of the container,
as a result, when the rear portion of the cassette which includes thus received spent lancet is engaged with the lancet ejecting device by inserting it into the lancet ejecting device through the opening at the front end of the lancet ejecting device over the predetermined length, the rear end of the lancet is located above and without abutting against the leading end of the lancet ejecting member.

15. The lancet assembly according to claim 9 wherein the lancet ejecting member comprises a stopper which prevents forward movement of the trigger member which initiates the ejection of the lancet except when the forward force acting on the lancet ejecting member acts on the rear end of the lancet while the rear end of the lancet is held with abutting against the leading end of the lancet ejecting member,
the stopper comprises a base portion and an oblique portion which is inclined with respect to the base portion, and front ends of these two portions are connected together while rear ends thereof are separated such that when a force is applied to the oblique portion, the stopper deforms elastically so that the rear end of the oblique portion and rear end of the base portion which are separated get closer to each other and when such force is removed, the stopper elastically revert to its original form,
in the case in which the stoppers in its original form, the rear end of the oblique portion abuts against a being locked portion of the trigger member which tries to move forward so as to prevent the forward movement,
when the rear portion of the cassette is fitted into the lancet ejecting device through the opening at its front end so that the lancet ejecting member is moved back while the rear end of the lancet abuts against the leading end of the lancet ejecting member, the leading end of the lancet ejecting member moved back moves on the oblique portion of the stopper, so that the leading end of the lancet ejecting member applies a force onto the oblique portion of the stopper, whereby the stopper elastically deforms and makes the rear end of the base portion and the rear end of the oblique portion which have been separated get closer to each other, and
as a result, the trigger member which tries to move forward is able to move forward while passing over the oblique portion with the being locked portion of the trigger member not abutting against the rear end of the oblique portion.

16. The lancet assembly according to claim 9 wherein the lancet ejecting device further comprises an electronic device which carries out a predetermined measurement with respect to an amount of blood which bleeds due to pricking.

17. (canceled)

18. A lancet ejecting device comprising a lancet ejecting member for ejecting a lancet of a lancet cassette according to claim 1, said lancet ejecting member being adapted for use in a lancet assembly including the lancet cassette.
